SEQUENCE LISTING (471 sequences)

SEQ ID NO: 1 - DNA, 409 bp, human
ctgagtgacagctggtggcagcactccttgctggggggcactgttcaacattcctctgccgtcggtgacccctagcccttctgactccttccagctttttcccagctttcccactgagcttctccagtcatgctcttctgacgtgactctctgagcagaactgagctttccaggcctctatggaatcctgcagatccagtggctgcagcttcaatcccagtgctgcaatcacacatccattctgcctggggaccctggagcctacttgtgcgctttgcatttcattgattgacgcctcccttcaacaagcatttactgaggcgcctactatgtactaatgctagatgttagatgtacaaagaagacagttttcatcctctaggaactcataggctaatggtgagacaca

SEQ ID NO: 2 - DNA, 511 bp, human
tggagacccctgaactcattgagctgaggaagaagaagattgaggaggcgatggacggaagtgagacacctcagctcttcactgtgttgccagagaagagaacagccactgttggaggggccatgatgggatcaacccacatttatgacatgtccacggttatgagccggaagggcccggctcctgagctgcaaggtgtggaagtggcgctggcgcctgaagagttggagctggatcctatggccatgacccagaagtatgaggagcatgtgcgggagcagcaggctcaagtagagaaggaggacttcagtgacatggtggctgagcacgctgccaaacagaagcaaaaaaaacggaaagctcagccccaggacagccgtgggggcagcaagaaatataaggagttcaagttttaggtcccctcacactagccctttttttggccctacgtctggatgcctgggcttcacacaagaaccacctctcccgcagttcccaaggacttgtcatt

SEQ ID NO: 3 - DNA, 425 bp, human
gtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaa

SEQ ID NO: 4 - DNA, 439 bp, human
  misc_feature (268)..(269): n is a, c, g, or t
gggtggtgcagttctagcggatatcatgaaagacaaagacaacttttggatgacccgacaagagtaccaagaaaagggtgtccgtgtgctagagaaacttggtgtgactgttcgataaactccaaagcttgttcccatcatacccgtaatgctttcttttttcctttattgccaatctttgaactcattcaactccaggacatggaagaggcctctctctgccctttgactggaaaggtcaagttttattctggtgtcttggggaagnnttgttaaatttttgttaatgtgggtaaatctgagtttaattcaactgcttccctanatagactagagggctaaggattctgtctgctgctttgtttcttctaagtaggcatttagatcattcctgtaggcttcctattttcactttactgctctaatgctgctagtcgta

SEQ ID NO: 5 - DNA, 312 bp, human
  misc_feature (106)..(106): n is a, c, g, or t
gaacagataagtttgcctgcatgctggacatgcctcagaaccatgaatagcccgtactagatcttgggaacatggatcttagagtcactttggaataagttcttanataaatacccccagccttttgagaacggggcttgttaaaggacgcgtatgtagggcccgtacctactggcagttgggttcagggaaatgggattgacttggccttcaggctcctttggtcataattttaaaatatgggagtagaaaacaacaaagaatggaatggactcttaaaacaatgaaagagcatttatcgtttgtcccttg

SEQ ID NO: 6 - DNA, 165 bp, human
gggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactt

SEQ ID NO: 7 - DNA, 511 bp, human
aacactgcataacccgtttctttgaggagtgtgaccccaacaaggataagcacatcaccctgaaggagtggggccactgctttggaattaaagaagaggacatagatgaaaatctcttgttttgaacgaagattttaaagaactcaactttccagcatcctcctctgttctaaccacttcagaaatatatgcagctgtgatacttgtagatttatatttagcaaaatgttagcatgtatgacaagacaatgagagtaattgcttgacaacaacctatgcaccaggtatttaacattaactttggaaacaaaaatgtacaattaagtaaagtcaacatatgcaaaatactgtacattgtgaacagaagtttaattcatagtaatttcactctctgcattgacttatgagataattaatgattaaactattaatgataaaaataatgcatttgtattgttcataatatcatgtgcacttcaagaaaatggaatgctactcttttgtggtttac

SEQ ID NO: 8 - DNA, 215 bp, human
ttctgagtgtagtgtggtaggacccggcgggtgtgcagcaactgccctggagccccagcccctgcgtccatctgtgctgtgcgccccacagtagacgtgcagacgtccctgagaggttcttgaagatgtttatttatattgtccttttttactggaagacgtacgcatactccatcgatgttgtatttgcagtggctgaggaattcttgtacgca

SEQ ID NO: 9 - DNA, 383 bp, human
gttctgatcttgtcaaacccagcctgaccgcttctgacgccgggatggcctcgttactagacttttctttttaaggaagtgctgtttttttttgagggttttcaaaacattttgaaaagcatttacttttttgaccacgagccatgagttttcaaaaaaatcgggggttgtgtgggtttttggtttttgttttagtttttggttgcgttgccttttttttttagtggggttggccccatgaagtgggtgccccactcacttctctgagatcgaacggactgtgaatccgctctttgtcggaagctgagcaagctgtggcttttttccaactccgtgtgacgtttctgagtgtagtgtggtaggacccggcgggtgtgcagcaa

SEQ ID NO: 10 - DNA, 431 bp, human
aggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgc

SEQ ID NO: 11 - DNA, 544 bp, human
cgagtggtgacaggacgctgaccgtggagcttaagccactgcctctccctctggtcccacaaatgggcgcccccccctccccatgcaggtggtgtcgggcccttcttgctgccctgccccaagttgggggtcagtgctgcctgtcccatgcttaacataaccgcctagctgctgtcacatttttcttgttttgtccttttatttttttctaataacctaaaaactggcaaaatagttctgcaggttgaagccatgtctacatgaaagtcctcagtaagtgttagagggaacagggcggagatatccttatgccacccccgctggaggatgtgggcagcttagggccctggaggcggtgcggcagggaagaggggtgcagaggctgtggctggtgagccggtcaggcacacaaggggcccttggagcgtggactggttggttttgccattttgttgtgggtatgctgcttttcttttctaaccaagaggctggttttggcatctctgtcccattccctgggatctggtggtcagccctaggataa

SEQ ID NO: 12 - DNA, 136 bp, human
cagttggcacttctgtctagaaaataataattgcaagttgtataactcaggcgtctttgcatttctgcgaaagatcaaggtctgcaagggaattcttgtgtgctgctttccatttgacaccgcagttctgttcagc

SEQ ID NO: 13 - DNA, 466 bp, human
atcgtggagctgtaggtgccttattggtttatgacattgctaaacatctcacatatgaaaatgtagagcgatggctgaaagaactgagagatcatgctgatagtaacattgttatcatgcttgtgggcaataagagtgatctacgtcatctcagggcagttcctacagatgaagcaagagcttttgcagaaaagaatggtttgtcattcattgaaacttcggccctagactctacaaatgtagaagctgcttttcagacaattttaacagagatttaccgcattgtttctcagaagcaaatgtcagacagacgcgaaaatgacatgtctccaagcaacaatgtggttcctattcatgttccaccaaccactgaaaacaagccaaaggtgcagtgctgtcagaacatctaaggcatttctcttctcccctagaaggctgtgtatagtccatttcccaggtctgagat

SEQ ID NO: 14 - DNA, 546 bp, human
tacttttctttctaacatatcaatgcttagcagaactattcagattgtcagtagtaaatttaaagacaaatgcccgttttcctccagtccatgaaacataccatacttatatacctgcaactaagtgtttaaaattatgctctgtaactctgtactgctagtattagaactaaaaatcttaaaatacagccagtgcttaatgcttatatcaatgtggatttgtcggcttttatgtaatctgtaatatgtatagcaggaaatacgaagagttacacagtgtatgccttaaaaggctgtttcttaaaggtgttacaaggggataatggtatttcaactagttatcagcaagtgacaatacattccaccacaaatacactcttgttcttctagcttttagactatatgaaaaaaccgggtgcttcaaagtacatgataagggaacactatacctgtcatggatgaactgaagactttgcctgttcattttttaaatattattttcaggtcctttgcttaccaaaggaggcccaatttcactcaaatgtt

SEQ ID NO: 15 - DNA, 469 bp, human
  misc_feature (36)..(36): n is a, c, g, or t
aagcatgctcagaccttcattgctctgtgtgccacngactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtgaggatgccagttgggccgaaagagagagacgcgtccataatctggtctcttcttctttctggttgtttttgttctttgtgttttagggtgaaacttaaaaaaaaaattctgcccccacctagatcatatttaaa

SEQ ID NO: 16 - DNA, 530 bp, human
gaacctcacagggggcttctgataccctcaaacatggagaacagtaagggagcagagtggttaaggactttcaggaacttaactattctggaataaggaatgaatcaactgaccttgggccagcaggtttttaactaaattgttacttgcctttctcacccagttaatcagtctctgtacttgtttccctttttgaaacaagtgtcttggttaactaattctgttttatggttgtgctaaattcatagcaggtgccttattctttgcttttagtcaaaccattccatatcagaattttccttggtttactatagatatttggctttaagttgttgtttgtgttttttaatgtacaatgttctgataaatttgactgttaaattgctatagctagcaatcattttacatatgtaaaaaattgcattccctttgtatttcatgtgtaattcaccaattaagtgcagtttatattcaggttggattatgcatgtttaggtaaacgaaagctgtgtcttacttgatttattctt

SEQ ID NO: 17 - DNA, 366 bp, human
  misc_feature (107)..(107): n is a, c, g, or t
cttgatttgattgcagttttttcctaattataacaaatttttcctcattggcctgtttttaatcctgtgcctagaaggagtacaaaatgcacactttacaaaattgnatatttaacacttacccactcccctttccccatctcttctaccgctcttgttgatcgtggtatctgatcttgactagataggctgaaggcacatggttccctccaaaaaccactattgataccactacaaaaacaagccagcaaaaagatactgtagagaggttggcttgcttccctctcttcctaactgcatgttgaaaaataagccgttattgatcttaaacatcggtcagatgagtcatacattgggttatttttt

SEQ ID NO: 18 - DNA, 530 bp, human
gaagtatacacttccgctgtaccacgcaatgatgggtggcagtgaggtggcccagaccctcgccaaggagacttttgcatccaccgcctcccagctccacagcaatgttgtcaactatgtccagcagatcgtggcacccaagggcagttagaggctcgtgtgcatggcccctgcctcttcaggctctccaggctttcagaataattgtttgttcccaaattcctgttcccgtgatcaatttcctggagtttatatcccctcaggataatctattctctagcttaggtatctgtgactcttgggcctctgctctggtgggaacttacttctctatagcccactgagccccgagacagagaacctgcccacagctctccccgctacaggctgcaggcacgcagggcagcgggtattctcctcccacctaagtctctgggaagaagtggagaggactgatgctcttcttttttctctttctgtcctttttcttgctgatttatgcaaagggctggcattctgatgcttttcag

SEQ ID NO: 19 - DNA, 473 bp, human
caccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagcca

SEQ ID NO: 20 - DNA, 516 bp, human
atctatttagagcttcaccatggcaatatgtatttcccttaaaacactgcaaacaaatatactaggagtgtgcccttttaatctttactagttattgtgagattgctgtgtaagctaataaacacatttgtaaatacattgtttgcaggacgaaaacttctgagttacagctcaggaaaagcctgctgaatttatgttgtaagcattacttaacacagtataaagatgaaaagacaacaaaaatatcttcatacttcctcatcccctcattggaacaaaaaccttaaactgggagaaccttagtcccctctctttcctcttcctcctccacttcccacttattgtcaccttgtaatattcagagagcacttggattatggatctgaatagagaaatgcttacagataatcattagcccacataccagtaacttatacttaaagatgggatggagttgtaaagtgcttttataatacaatataattgttaaaggcaagggttgactctttgttttat

SEQ ID NO: 21 - DNA, 322 bp, human
cacacccgtttaaggatttcacatcatacaaagcgcttgcttagatggcttctatcctaggcatatgctggccgggtgctctacatataaattctcattgtatcctcccatctgtccactgaggaagattatcaaatggatcttcatccaatggatgcataaactttcctacttacttgtagtggcaaagctggctttcaagtacaagtttgttggctccattacctatgctcctattatccgcttctgtcccgcaacaaagtagctcacttaggcgtatgaccacatgcattatgatagtttcccaccaccatattgaata

SEQ ID NO: 22 - DNA, 461 bp, human
agcccacagtgcctgtacaggaaggtgcctggccatgtcacctggctgctaggccagagccatgccaggctgcgtccctccgagcttgggataaagcaaggggaccttggcgctctcagctttccctgccacatccagcttgttgtcccaatgaaatactgagatgctgggctgtctctcccttccaggaatgctgggcccccagcctggccagacaagaagactgtcaggaagggtcggagtctgtaaaaccagcatacagtttggcttttttcacattgatcatttttatatgaaataaaaagatcctgcatttatggtgtagttctgagtcctgagacttttctgcgtgatggctatgccttgcacacaggtgttggtgatggggctgttgagatgcctgttgaaggtacatcgtttgcaaatgtgagtttcctctcctgtccgtgtttgtttagtac

SEQ ID NO: 23 - DNA, 554 bp, human
gttcagattaactgcttatagccttagaaagccttttacaaaattaaaaaaaaaatagatgtgcattcagtttttaagaatggaatcatccaaaggaattcctttttttgaggtttggatgttgcagctagtaaaggatatttttgctctgttcagcagttctaaaaattgctgaagtaggggccaggtcactggtagttatagtatggaatgggagaagtgaaagttcagttatagaactttccatacttccaagtttactgcaagtttttatgcttgagagagatgctttctaatataagactgatgtgttgattttactgattgtactgtacatctattaaagccttagattattacattacgggttggaacccataccaatgtaatttcaatcgtgttaagaaagtaatggtgacttcacatgttattgtagttagttacattatagaatattacttatttttcttgttaaaatgtagtttttcatttcctacatttattagattttcattttctattaacaattgaataccatttcagtttatagactt

SEQ ID NO: 24 - DNA, 521 bp, human
gaaatccctgagtatcaggccttgttataaataagctgcataatcaataaatagaacaagggactttttgttgataatccaaatactcaaagtttacgtaatgaaaattatagcgtgtgtgcaaactcttgagggttgattatgctgcaatttagcatgttggaacgtctagggagaaggttgactttttgcacttctgtatatagtcaaaagagagaaacctgtataatagtaagatcttattttgaataaaaacgtctataattacaaggagttttgttaaggctaatacaatgacagactgagcaaaattgcttgcaaaagtggcacagagttagcactccataccccttcaaacatgttgctttgctttcttgtggacagcttgtagtttgccaggattttttcagctggaaagatacgccatcctttcaaaccctcatgactgacaaaaactccatggggccaaatctgcctgaagatcattaccaaaaatagcaggtacttctaccattaaggtg

SEQ ID NO: 25 - DNA, 434 bp, human
cggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagccc

SEQ ID NO: 26 - DNA, 491 bp, human
ttcaattccatcctaaaactccttttaaccaagcttagcttctcaaaggcctaaccaagccttggcaccgccagatcctttctgtaggctaattcctcttgcccaacggcatatggagtgtccttattgctaaaaaggattccgtctccttcaaagaagttttatttttggtccagagtacttgttttcccgatgtgtccagccagctccgcagcagcttttcaagatgcactatgcctgattgctgatcgtgttttaactttttcttttcctgtttttattttggtattaagtcgttgcctttatttgtaaagctgttataaatatatattatataaatatattaaaaaggaaaatgtttcagatgtttatttgtataattacttgattcacacagtgagaaaaaatgaatgtattcctgtttttgaagagaagaataatttttttttctctagggagaggtacagtgtttatattttggagccttcctg

SEQ ID NO: 27 - DNA, 474 bp, human
gaattgtcaggcaccctacaggaaggcctgccatgcttgtggccaactgtttcactggagcaaagaaagagatctcataggacggagggggaaaatggttttccctccaagcttgggtcagtgtgttaactgcttatcagctattcagacatctccatggtttctccatgaaactctgtggtttcatcattccttcttagttgacctgcacagcttggttagacctagatttaaccctaaggtaagatgctggggtatagaacgctaagaattttcccccaaggactcttgcttcctcaagcccttctggcttcgtttatggtcttcattaaaagtataagcctaactttgtcgctagtcctaaggagaaacctttaaccacaaagtttttatcattgaagacaatattgaacaaccccctattttgtggggattgagaaggggtgaatagaggcttgagactttcctttgtgt

SEQ ID NO: 28 - DNA, 398 bp, human
ccctttcctggatttctaaactgctcaattttgactcaaaggtgctatttaccaaacactctccctacccattcctgccagctctgcctccttttcaactctccacattttgtattgccttcccagacctgcttccagtctttattgctttaaagttcactttgggcccacagacccaagagctaattttctggtttgtgggttgaaacaaagctgtgaatcactgcaggctgtgttcttgcatcttgtctgcaaacaggtccctgcctttttagaagcagcctcatggtctcatgcttaatcttgtctctcttctcttctttatgatgttcactttaaaaacaacaaaacccctgagctggactgttgagcaggcctgtctctcctattaagtaaaa

SEQ ID NO: 29 - DNA, 381 bp, human
catccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgccccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactc

SEQ ID NO: 30 - DNA, 488 bp, human
gttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttca

SEQ ID NO: 31 - DNA, 534 bp, human
  misc_feature (306)..(306): n is a, c, g, or t
atgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacncccgtcctgcctgtgcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacag

SEQ ID NO: 32 - DNA, 527 bp, human
gaggagcttatatttggaaccgaccatattacaacaggtgcttccagtgattttgataatgccactaaaatagcaaagcggatggttaccaaatttggaatgagtgaaaagcttggagttatgacctacagtgatacagggaaactaagtccagaaacccaatctgccatcgaacaagaaataagaatccttctaagggactcatatgaacgagcaaaacatatcttgaaaactcatgcaaaggagcataagaatctcgcagaagctttattgacctatgagactttgatgccaaagagattcaaattgttcttgaggggaaaaagttggaagtgagatgataactctcttgatatggatgcttgctggttttattgcaagaatataagtagcattgcagtagtctacttttacaacgctttcccctcattcttgatgtggtgtaattgaagggtgtgaaatgctttgtcaatcatttgtcacatttatccagtttgggttattctcattatgacacctattgcaaa

SEQ ID NO: 33 - DNA, 440 bp, human
  misc_feature (30)..(31): n is a, c, g, or t
ctcacattttgcgagattatcctgatgganntgnnnnnccatgatgcagcctggcctttcctagagcctgtgaacccacgtttggtgagtgggtaccggcgcatcatcaaaaatcctatggatttttccaccatgcgggagcggctgctcaggggagggtacaccagctcagaggagtttgcggctgatgccctcctggtatttgacaactgccagactttcaacgaggatgactctgaagtaggcaaggctgggcacatcatgcgccgcttcttcgagagccgctgggaggagttttatcagggaaaacaggccaatctgtgaggcaagggaggtggggagtcaccttgtggcatctccccccaccttccaaacaaaaacctgccattttcacctgctgatgctgccctgggtccagactcaagtcagatacaaccctg

SEQ ID NO: 34 - DNA, 447 bp, human
gagcagtcactctcagaatcttgattccccatcagccaaagcaaaagatggctgctgctttgtaggcatgtgcctgcaagtgggaccttgctgggcattatatgccctgtgggggtttcagagaccctgaaagaggagggaggacccgcctccttgtctgcacaactgcatgcacttctctccccatcgctccacaacctgaaaccgagaaggagttgctgaccagtgcccaccccggcagcccgggaggaacacaggcagctcctttcccttcacgtggtctgcagagagcagggtgagctgccagctgcccctctccaccagggtaccctgtcttggtggttaggggccacttttcctttgaggctctagtggaggtggatgtccttctctgccaggcttggcacatgatgtgaagaataaatgcccaattcttactgttcaggt

SEQ ID NO: 35 - DNA, 530 bp, human
ggggtggctccaagcattaagaggtttaatctttgatggcattgttctagttttgaaatttctagtatatttcagagtctcttagaagacttgtgtgggaagtttcactttgttttcagtgaagatcacaaacctccttcttcctttactcaagaggaaaggtcccagtatacatatttgaatggttgatggttttcaagaccttcagggagctccctgcattttacctagaaacagaaaaggcccgcaaaatcttaagtttcctggcctgcatttcccgggtaggggcaaatgactccaagctggtctctaagccaatacccttataaaccagagcccaggaaagacagctcgagtgtataattctctggagctcaattctatgcagttgtgctgatatttcattaagtcactgtgtatttttaagtgttgatacattaaaagtcgctttatggaagatgagtaaattttttaaatacttggaaattttatttccttgttaacttctacagatcagggcatgcaaccaa

SEQ ID NO: 36 - DNA, 503 bp, human
tagatttcttctggcttatgtttctactttaggaccctcattgttctcttattaaaaaaaattatttcctgtgcatctcatggactgcagggtaaattatttgggcataaataatttaaatagttttctttcattttgactatctccagtaataacagtttttattatccagcatattggcttattgcacaaatcttaaaatgtacattgactactttctgagaagaaagtggtatcagtactcatgatgaaaaggttactactgaacaaattcacatttcaggaacacctctatctttggtttaaatcttactcttagtttttccgtctaaaaatcatactggtattagtatcaggtaaggaaattaaagtttttaaaatggtttcattctctgcaatatgcaaaatttagattttactttctggtactgtaaagaacctgaagtgatttacacttaatgggtgattaatccagtattctttaccctgaatgtttggatatt

SEQ ID NO: 37 - DNA, 452 bp, human
atgagattcacattctagtctttattttcctcctgttttgtccctgtgcttgtacatcttccttttatttgtgtgttatagttctattccatttgagaaggcagttggtaagaactagattgcatgtacaaagacaggtttactaagtgctgtacagtggtcctgaggttacagttgaattagaaaaacgaaatgtacttacaggaaataagaaagcaaacctttcaaatgagagtgatgatttctttaaaaaaaatcagtttttttctctcaaataatgttctttatttcacgaaatcgtcaatcttaagcatgagcagggataaacaactcctagaaggaactcaattcattcttcctggattttctctgttgttaaatcacaaaaatgatagtccccaatcgtttctttataggaggttattacatttcattacagtcactgcattttg

SEQ ID NO: 38 - DNA, 510 bp, human
gctcaagggtaggtgggcgtgggtggtaggagggttttttttacccagggctctggaaggacaccaaactgttctgcttgttaccttccctcccgtcttctcctcgcctttcacagtcccctcctgcctgctcctgtccagccaggtctaccacccaccccacccctctttctccggctccctgcccctccagattgcctggtgatctattttgtttccttttgtgtttctttttctgttttgagtgtctttctttgcaggtttctgtagccggaagatctccgttccgctcccagcggctccagtgtaaattccccttccccctggggaaatgcactaccttgttttggggggtttaggggtgtttttgtttttcagttgttttgtttttttgttttttttttttcctttgccttttttcccttttatttggagggaatgggaggaagtgggaacagggaggtgggaggtggattttgtttatttttttagctcatttccaggggtggg

SEQ ID NO: 39 - DNA, 544 bp, human
cagttcccatgtgagccattcaaatttttggagccaactctaagactagaatattgtgaagcattggctatgcttagggaagctggagtcgaaatgggagatgaagacgatctgagcacaccaaatgaaaagctgttgggtcatttggtaaaggaaaagtatgatacagatttttatattcttgataaatatccattggctgtaagacctttctataccatgcctgacccaagaaatcccaaacagtccaactcttacgatatgttcatgagaggagaagaaatattgtcaggagctcaaagaatacatgatcctcaactgctaacagagagagctttacatcatggaattgatttggagaaaattaaggcttacattgattccttccgctttggagcccctcctcatgctggtggaggcattggattggaacgagttactatgctgtttctgggattgcataatgttcgtcagacctccatgttccctcgtgatcccaaacgactcactccttaaattcacactttgccacttaactccagtg

SEQ ID NO: 40 - DNA, 436 bp, human
gagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcaga

SEQ ID NO: 41 - DNA, 484 bp, human
ggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttccc

SEQ ID NO: 42 - DNA, 510 bp, human
aaccatcacatatgagtctccacagattgatggcggggctggtggtgattcgggcacgttactgaccgcacaaaccatcacatctgagtccgtgtcaacaacgacaaccacacacatcaccaagactgtaaaaggtggaatttctgaaacaagaattgagaaacgcattgtgatcacaggagatggagatattgatcatgaccaggcactggctcaggcgatcagggaagccagagagcagcaccctgacatgtcggtcacaagagtggtggtacacaaagaaacagagttggctgaggaaggggaagattaagtaagaaagtcattttttaaacaacactcaactttgtgaacccctgaagattttttgaccgttccaagtcttaatgccacaccactattccagcgaatttatgctacaactggtaacaatgaccagaagcctgaagaattaaaatgccaacaccaaacctttccttaccagctctggtctatattgctcccatgcat

SEQ ID NO: 43 - DNA, 439 bp, human
ggtgtggctcattgactttaatccatttggtgaagtcacagattcactgctgttcacctgggaagaactgatatctgagaacaacttaaacggcgattttagtgaagttgacgctcaagagcaggattccccagctttccgttgcacaaacagtgaagtgacagtccagcccagcccctatttgagttaccggctacccaaggactttgtagacctctctactgggagggacgctcacaagctaatagacttccttaagctgaagagaaatcagcaggaggacgactgatgagcgtactgtaactggagaagaggaggccccgccccaccgctccgggagctgctcatcagccgcaacttcctgccgaccctgatgcgggtgggccgagcagtgtggacatcagccactttttatattcatgtacattcacctggggaa

SEQ ID NO: 44 - DNA, 537 bp, human
aaacctgtccagtgtgcaagcaaaaagttgttccttctcaaggcgattcagactctgacacagacagtagtcaagaagaaaatgaagtgacagaacatacccctttactgagacctttagcttctgtcagtgcccagtcatttggggctttatcggaatcccgctcacatcagaacatgacagaatcttcagactatgaggaagacgacaatgaagatactgacagtagtgatgcagaaaatgaaattaatgaacatgatgtcgtggtccagttgcagcctaatggtgaacgggattacaacatagcaaatactgtttgactttcagaagatgattggtttatttccctttaaaatgattaggtatatactgtaatttgattttttgctcccttcaaagatttctgtagaaataacttattttttagtattctacagtttaatcaaattactgaaacaggacttttgatctggtatttatctgccaagaatatacttcattcactaataatagactggtgctgtaactcaagcatca

SEQ ID NO: 45 - DNA, 364 bp, human
gggagaggcaacaaggtaattcagcctttcctcctatcagcacaaagaaactcaaagctgttttttccctttctgttccaaagcagtcttatcctgacaggagcggtctatactagtgcagatttcaacacttttttttaacgttttaattactatagtgttatgtagagatttgattgagcagctaatgtttctgaactttacttactaattttcagtgtccttaagggttctgtagtgttatcaaagcaaaaagaaaatgctgcataaaaataccaaacttcagcaactgttaatactcagatcatatacctcttaataaatagcatcttatgctaattagccctgctaaactatgtacaga

SEQ ID NO: 46 - DNA, 514 bp, human
tggagtttcagtctccaactctcagcccaccaatgaaagccactctatcaaagccatcctgaaaaatatctcagtcctggctttctctgtctgcttcatcttcactatcaccattgggatgtttccagccgtgactgttgaggtcaagtccagcatcgcaggcagcagcacctgggaacgttacttcattcctgtgtcctgtttcttgactttcaatatctttgactggttgggccggagcctcacagctgtattcatgtggcctgggaaggacagccgctggctgccaagcctggtgctggcccggctggtgtttgtgccactgctgctgctgtgcaacattaagccccgccgctacctgactgtggtcttcgagcacgatgcctggttcattttcttcatggctgcctttgccttctccaacggctacctcgccagcctctgcatgtgcttcgggcccaagaaagtgaagccagctgaggcagagaccgcaggagccatcatggccttcttc

SEQ ID NO: 47 - DNA, 549 bp, human
cggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccag

SEQ ID NO: 48 - DNA, 519 bp, human
tgtcagagtatgcacggcgctttggtgttccggtcattgctgatggaggaatccaaaatgtgggtcatattgcgaaagccttggcccttggggcctccacagtcatgatgggctctctcctggctgccaccactgaggcccctggtgaatacttcttttccgatgggatccggctaaagaaatatcgcggtatgggttctctcgatgccatggacaagcacctcagcagccagaacagatatttcagtgaagctgacaaaatcaaagtggcccagggagtgtctggtgctgtgcaggacaaagggtcaatccacaaatttgtcccttacctgattgctggcatccaacactcatgccaggacattggtgccaagagcttgacccaagtccgagccatgatgtactctggggagcttaagtttgagaagagaacgtcctcagcccaggtggaaggtggcgtccatagcctccattcgtatgagaagcggcttttctgaaaagggatccagcacacctcct

SEQ ID NO: 49 - DNA, 553 bp, human
agatccaccttcagaatggacatgattcagggagctagaagacactttaagttatactggaaaattcaggtgccactgaaagccagatttatagtattccatctttaatatgtgggactaacagcagtgtagattgttaccttaatattttttgctgggaccatctacctgccttatactacacttaggaaaaagtattacatatggtttattttgaaacttcaagtattattgccttaatgtctcttaaccctgttacacgctgcttgtagacatgttaatatagtaatacctttatgatatattgagtttaaggactaccctttttctgttttatcatgtattcattattttgtatatgtacagggcaagtaggtatataatttgataaagttgcaattgaaatattattaacagaagatgtaagaaatttctgcatggtctaaatctttgtgtactttatttgtaaattatttgccctggagttttagaaaatagtttctgaattttaaacttgctggattcatgcagccagctttgcaggttatcagag

SEQ ID NO: 50 - DNA, 493 bp, human
gagtggactcagctggatatgttttataggaattgggcacttaaggaaagcttcataaaagccattggtgttggactaggatttgaattgcagcggcttgaatttgatctatctccattaaacttggatataggccaagtttataaagaaacacgtttattcctggatggagaggaagaaaaagaatgggcatttgaggaaagcaaaatagatgagcaccattttgttgcagttgctcttaggaaacccgatggatctagacatcaggatgttccatctcaggatgattccaaaccaacccagaggcaatttactattctcaactttaatgatttaatgtcatctgccgttcccatgacacctgaagatccttcattttgggactgtttttgcttcacagaagaaattccaatacgaaatggtacaaagtcatgatgattccctgagtaacaaagggaaatgaaaactgtttgtgatcttccgtattcactga

SEQ ID NO: 51 - DNA, 446 bp, human
gtctgtcagctctcggaatagggacagtccttactggtgccccaaggtgggacttggagaatattttgcttggcatatgtttggtctgaatggtgtagttgctggttccctagagaggaaaaggtggcaggcccagctttgctgggaaatggctcttaatttccagttgaaaccctagtagaattgtgaatgaaaacctcaaggttgagcccctctgccaagcagcagagctagtagaaggggatgcaggggcaaagcactcagttgccaagcaaggaggagagatgtacgtgggctgtgtggcagtccccacaccctgccctggcttcttcaggttatcgcaccactatggaatcctttgcagaatggtactcatataatggtttaaaacaacacattcataattgactctgtgcaggatgtcactcaatcagtttgggtttgct

SEQ ID NO: 52 - DNA, 448 bp, human
aagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaa

SEQ ID NO: 53 - DNA, 421 bp, human
caactggggagttccgtgtggaccgtgaaggttctccagtgctgctcaactgcctcatgtacaagatgtgttactatcgctttggacaggtttacacagaagccaagcgtcctccaggctttgaccgtgtccgaaatgctgagattgggaataaagactttgagcttgatgtcctggaggaaggctataccacagaacattggctggtcaggatatacaaggtaaaggacctggataatcgaggcttgtcaaggacataaatgtcacgtccagctctgatatcttcgcactgagcacatcacatttaggacgttgaagattttttttttttttttttttttaatatgcagtttgtaagaacaaaactggatggcatccgaattgtctggaagttttgtcttgggcatgatgggctgggcca

SEQ ID NO: 54 - DNA, 537 bp, human
gacaccatcctgaatgaggggtccagcctgggggggactctaccaaggtcttcttgggctgggaaagcccatgtagggcaggccttctataagtgcgggcaccaagggcgcctacatccccaggccttgctggggtgcaggggtatatcaacttcccattagcaggagctccccagcggcaagcctggcccagtgggctcggtagtgcccagctggcaggcctgaggtgtacatagtccttcccggccatattaaccacacagcctgagcctggcccagcctcggctgccagaggtgcctttgctaggcccggagccgttgcccggccttgcccttgccctattcctctcctcctcctcctcctgggtcccccagggtggctgggcttgggctatgtgggtggtggtggcggggggtcttgggggcctctcagctcccgcccatgcctccctgatgggtgggcccagggcggcctctctctgaggagacctcacccactcctcgctcagtttgaccactgtaagtgcctgcactctg

SEQ ID NO: 55 - DNA, 468 bp, human
taggatcctagcctgtaaagacacagaagatgctgacctcacccctgccacctatcccaagacctcactggtctgtggacagcagcagaaatgtttgcaagataggccaaaatgagtacaaaagttctgttttccatcagacccagtgatgttgcgattcacacgttcaattcaagacctgaccgttagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattgggaattatgagcagaacaaatactcaactaaatgcacaaagtataaagtgtagccatgtctagacaccatgt

SEQ ID NO: 56 - DNA, 490 bp, human
atgagcccaagcacacatgcatggcatttgttcctgaacaggagggcatccctggggatgtggctggagcatgagccagctctgtcccaggatggtcccagcggatgctgccaggggcagtgaagtgtttaggtgaaggacaagtaggtaagaggacgccttcaggcaccacagataagcctgaaacagcctctccaagggttttcaccttagcaacaatgggagctgtgggagtgattttggccacactgtcaacatttgttagaaccagtcttttgaaagaaaagtatttccaacttgtcacttgccagtcactccgttttgcaaaaggtggcccttcactgtccattccaaatagcccacacgtgctctctgctggattctaaattatgtgaattttgccatattaaatcttcctcatttatactattatttgttacgttcaatcagaatccccgaaacctcctataaagcttagctgccccttctg

SEQ ID NO: 57 - DNA, 417 bp, human
aatgataaaacacctcacacctcactctttatagtgcacaaaatgaatgaggtctgggctaggtagaaaaagggtcaatgctatttttgtttttagaatcattaccttttaccagcttttaaccatctgatatctatagtagacacactatcatagttaacatagttaagttcagcacttgtctcattttaatgtaaagatttgcttccattttcctacaggcagtctctctcttcctcacagtcccactgtgcaggtgctattgttactcttacgaatattttcagtaatgttattttcttctaagtgaaatttctagcctgcactttgatgtcatgtgttccctttgtctttcaaactccaaggttcccctgtggccctctcccttaccctgggaaggcctcttggagaccttac

SEQ ID NO: 58 - DNA, 540 bp, human
atcagcccactccagatagctgcatcatcagcatggttgtgggccagcttaaggcggatgaagaccccatcatggggttccaccagatgttcctattaaagaacatcaacgatgcttgggtttgcaccaatgacatgttcaggctcgccctgcacaactttggctgacctcctctcagctaggcactcacgctgtttcctcctccctcctcttcccaatactattcccactcctccagatgctccaaatatcatgcacaaatgagcagggccgcggtgggagtgggcgcagtgcgctgctgccactgaggtgttgtgcatgatgtttggatgctagactagttgcatctgacgggagaagtttgtgttgtaccagcgcatgccttggaaagacttaagtaatgcaaaaggttgtccttttttttttttttttttttttaatctactgacaagttgctctagtaacccaaagaagtgaaggagaaagcagctgcctcaccgcccagacattgatttgttcagatgtttcaatgcctcatga

SEQ ID NO: 59 - DNA, 443 bp, human
tatatccccacgaaggccttcattacttacgtgctcctggctgggatggcactgggcattcagaaaaggttctccccggaggtgctgggcctgtgtgcaagcacagcgctggtgtgggtggtgatggaggtgctggccctgctcctgggcctctacctggccaccgtgcgcagtgacctgagcacctttcacctgctggcctacagtggctacaaatacgtgggaatgatcctcagtgtgctcacggggctgctgttcggcagcgatggctactacgtggcgctggcctggacctcatcggcgctcatgtacttcattgtgcgctctttgcggacagcagccctgggccccgacagcatggggggccccgtcccccggcagcgtctccagctctacctgactctgggagctgcagccttccagcccctcatcatatactggct

SEQ ID NO: 60 - DNA, 543 bp, human
taccctcagatcaccttcgagaacatgattgtggataacaccaccatgcagctggtgtcccggccccagcagtttgatgtcatggtgatgcccaatctctatggcaacatcgtcaacaatgtctgcgcgggactggtcgggggcccaggccttgtggctggggccaactatggccatgtgtacgcggtgtttgaaacagctacgaggaacaccggcaagagtatcgccaataagaacatcgccaaccccacggccaccctgctggccagctgcatgatgctggaccacctcaagctgcactcctatgccacctccatccgtaaggctgtcctggcatccatggacaatgagaatatgcacactccggacatcgggggccagggcacaacatctgaagccatccaggacgtcatccgccacatccgcgtcatcaacggccgggccgtggaggcctaggctggccctaggaccttcttggtttgctccttggattccccttcccactccagcaccccagccagcctggtacgcagatcccagaat

SEQ ID NO: 61 - DNA, 433 bp, human
gccagcgctctgacatgcagaaggtgaccctgggcctgcttgtgttcctggcaggctttcctgtcctggacgccaatgacctagaagataaaaacagtcctttctactatgactggcacagcctccaggttggcgggctcatctgcgctggggttctgtgcgccatgggcatcatcatcgtcatgagtgcaaaatgcaaatgcaagtttggccagaagtccggtcaccatccaggggagactccacctctcatcaccccaggctcagcccaaagctgatgaggacagaccagctgaaattgggtggaggaccgttctctgtccccaggtcctgtctctgcacagaaacttgaactccaggatggaattcttcctcctctgctgggactcctttgcatggcagggcctcatctcacctctcgcaagagggtctc

SEQ ID NO: 62 - DNA, 502 bp, human
catccagcagctggcatctgaccttggcactcgaactgacctcaagctcagctacctggaagaggccgtgatgcacctggaccacagtgaccccatcactcgggaccacatgggctccgttatggcccaggtgcgccaaaagctttttcagttcctgcaggctgagccacacaactcacttggcaaagcagctcggcgtctcagcctcatgctgcatggcctcgtgacccccagcctcccttagctgctaagcctgccttgcccaggggtgggatggcactgaaggccagcagacaggcctaggctggggcagggtcacggctggcctttacctgctcaggcccccatctctggggtgtttgggggtcagggagcagggagcactggccgtggtctacagcgtgtggtagtcagaaggtttagctgggcccagggcaggtattgcgcctgcttgggttctgccatgcctggagcatgaccctgagatcgtgacaccacttga

SEQ ID NO: 63 - DNA, 524 bp, human
tggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttgtgaaatttgagaatttgtaagactaattatggtaacttagcttagtattcaatataatgcattgtttggtttcttttaccaaattaagtgtctagttcttgctaaaatcaagtcattgcattgtgttctaattacaagtatgttgtatttgagatttgcttagattgttgtactgctgccatttttattggtgtttgattattggaatggtgccatattgtcactccttc

SEQ ID NO: 64 - DNA, 380 bp, human
ggaaacctctcagtgtcttgacatcaccctacccaggcggtgggtctccaccacagccactttgagtctgtggtccctggagggtggcttctcctgactggcaggatgaccttagccaagatattcctctgttccctctgctgagataaagaattcccttaacatgatataatccacccatgcaaatagctactggcccagctaccatttaccatttgcctacagaatttcattcagtctacactttggcattctctctggcgatggagtgtggctgggctgaccgcaaaaggtgccttacacactgcccccaccctcagccgttgccccatcagaggctgcctcctccttctgattaccccccatgttgcatatcaggg

SEQ ID NO: 65 - DNA, 473 bp, human
aagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacaga

SEQ ID NO: 66 - DNA, 359 bp, human
gttgtttgacgattctgatgagagggggactttgggtggttttgggagtgttgaagaagggcccctatccactggcagcagctttattctcagtagcgatgatgatgacgatgatatttaatcccttaaacttgctttttagggagagtcctccatctacatttgcctgtgcttcagggtaattactagtagtgttacatgaacatgtgcatagtggtaggatgccatcagattaaagcattgaagtgtttcattgttacctgtacctaatggttttaaatatatgttaattgattgtttagttaaaatgtcatagttacaatgcaagtaaactggatacttgttcttttgtcagattt

SEQ ID NO: 67 - DNA, 549 bp, human
  misc_feature (76)..(76): n is a, c, g, or t
aaactactaaccactgcaagctcttgtcaaattttagtttaattggcattgcttgttttttgaaactgaaattacntgagtttcattttttctttgaatttatagggtttagatttctgaaagcagcatgaatatatcacctaacatcctgacaataaattccatccgttgttttttttgtttgtttgttttttcttttcctttaagtaagctctttattcatcttatggtgcagcaattttaaaatttgaaatattttaaattgtttttgaactttttgtgtaaaatatatcagatctcaacattgttggtttcttttgtttttcattttgtacaactttcttgaatttagaaattacatctttgcagttctgttaggtgctctgtaattaacctgacttatatgtgaacaattttcatgagacagtcatttttaactaatgcagtgattctttctcactactatctgtattgtggaatgcacaaaattgtgtaggtgctgaatgctgtaaggagtttaggttgtatgaattctacaaccctataata

SEQ ID NO: 68 - DNA, 533 bp, human
tgcaatcattgatgtgcctgtccccagtttctctgatagtgaccctgcagcaattattcatgactttgaaaagggaatgactgcttacctggacttgttgctggggaactgctatctgatgcccctcaatacttctattgttatgcctccaaaaaatctggtagagctctttggcaaactggcgagtggcagatatctgcctcaaacttatgtggttcgagaagacctagttgctgtggaggaaattcgtgatgttagtaaccttggcatctttatttaccaactttgcaataacagaaagtccttccgccttcgtcgcagagacctcttgctgggtttcaacaaacgtgccattgataaatgctggaagattagacacttccccaacgaatttattgttgagaccaagatctgtcaagagtaagaggcaacagatagagtgtccttggtaataagaagtcagagatttacaatatgactttaacattaaggtttatgggatactcaagatatttactcatgcatttactcta

SEQ ID NO: 69 - DNA, 508 bp, human
aagtgttgcacagtcttccagcagttcggcagtatctgttttcactctatgaatgccgttactctgttttcttccaatcattagcggttgtggaacaggaaatgaaaaaggactggctttttgcccctcattatcgatactatgtaagagaaatgagaattcatgcatacagtcagctgctggaatcatataggtcattaacccttggctatatggcagaagcgtttggtgttggtgtggaattcattgatcaggaactgtccaggtttattgctgccgggagactacactgcaaaatagataaagtgaatgaaatagtagaaaccaacagacctgatagcaagaactggcagtaccaagaaactatcaagaaaggagatctgctactaaacagagttcaaaaactttccagagtaattaatatgtaaagccatgtaactaacaaaggatttgctttagagataattatttggaatttttatagcttacttcacaatgtgcccaggtc

SEQ ID NO: 70 - DNA, 430 bp, human
gtgatgaccgctgctggaacgggatggccagaggccggtacctccccgaggtcatgggtgacggcctggccaaccagatcaacaaccccgaggtggaggtggacatcaccaagccggacatgaccatccggcagcagatcatgcagctgaagatcatgaccaaccggctgcgcagcgcctacaacggcaacgacgtggacttccaggacgccagtgacgacggcagcggctcgggcagcggtgatggctgtctggatgacctctgcggccggaaggtcagcaggaagagctccagctcccggacgcccttgacccatgccctcccaggcctgtcagagcaggaaggacagaagacctcggctgccagctgcccccagcccccgaccttcctcctgcccctcctcctcttcctggcccttacagtagccag

SEQ ID NO: 71 - DNA, 363 bp, human
gtcccgcacagtggacggaggtccccggttgctggtcaggtccccatggcttgttctctggaacctgactttagatgttttgggatcaggagcccccaacacaggcaagtccaccccataataaccctgccagtgccagggtgggctggggactctggcacagtgatgccgggcgccaggacagcagcactcccgctgcacacagacggcctaggggtggcgctcagaccccaccctacgctcatctctggaaggggcagccctgagtggtcactggtcagggcagtggccaagcctgctgtgtccttcctccacaaggtccccccaccgctcagtgtcagcgggtgacgtgtgttcttttga

SEQ ID NO: 72 - DNA, 464 bp, human
ctacccttatgatgacccattttccctcatgaccgatcccaagctcatcatttggagccctgtgcgccgctcagatgtggcctggaactttgagaagttcctcatagggccggagggagagcccttccgacgctacagccgcaccttcccaaccatcaacattgagcctgacatcaagcgcctccttaaagttgccatatagatgtgaactgctcaacacacagatctcctactccatccagtcctgaggagccttaggatgcagcatgccttcaggagacactgctggacctcagcattcccttgatatcagtccccttcactgcagagccttgcctttcccctctgcctgtttccttttcctctcccaaccctctggttggtgattcaacttgggctccaagacttgggtaagctctgggccttcacagaatgatggcaccttcctaaaccctcatgggtgg

SEQ ID NO: 73 - DNA, 380 bp, human
gaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaact

SEQ ID NO: 74 - DNA, 529 bp, human
gagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagta

SEQ ID NO: 75 - DNA, 510 bp, human
agagcagatttccactgcaggcaccgaagcctcggggacaggcaatatgaagttcatgctaaatggggccctaactatcgggaccatggatggggccaatgtggaaatggcagaagaagctggggaagagaacctgttcatctttggcatgaggatagatgatgtggctgctttggacaagaaagggtacgaggcaaaagaatactatgaggcacttccagagctgaagctggtcattgatcaaattgacaatggctttttttctcccaagcagcctgacctcttcaaagatatcatcaacatgctattttatcatgacaggtttaaagtctttgcagactacgaagcctatgtcaagtgtcaagataaagtgagtcagctgtacatgaatccaaaggcctggaacacaatggtactcaaaaacatagctgcctcggggaaattctccagtgaccgaacaattaaagaatatgcccaaaacatctggaacgtggaaccttcagatctaaa

SEQ ID NO: 76 - DNA, 335 bp, human
gttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggc

SEQ ID NO: 77 - DNA, 479 bp, human
caaaggacaggcggagaccggccgtcatacgcgagcctcatgaaagaaagatccttgcactgctggatgctctgagtacggtgcatagtcagaagatgaagaaggccaaggagcagcggcacctgcacaataaagagcacttcagagccaagcagaaggaggaggaggagaagctgaagcggcagaaggacctcaggaagaagctcttcagaattcaggggcagaaggaaagaagaaaccagaagtccagtttgaagggggctgagggccaattgcagtgagcctttggactggagggactgtccctggatctgcggaggtagacagtttcaaacatcacagtttgaatgcctgtgaatgacacgtcagtgggaaagagctcaagagatgtctctactcaaactgtgcctgcaggaggaggaacagagaagcctgggctgctgggactgggttcattctcatgacttggggctgtcgag

SEQ ID NO: 78 - DNA, 542 bp, human
gcagccacctacaagtatgtgaacatgcaggatcccgagatggacatgaagtcagtgactgaccgggcagcccgcaccctgctgtggactgagctcttccgaggcctgggcatgaccctgagctacctgttccgggaaccggccaccatcaactacccgttcgagaagggcccgctgagccctcgcttccgtggggagcatgcgctgcgccggtacccatccggggaggagcgttgcattgcctgcaagctctgcgaggccatctgccccgcccaggccatcaccatcgaggctgagccaagagctgatggcagccgccggaccacccgctatgacatcgacatgaccaagtgcatctactgcggcttctgccaggaggcctgtcccgtggatgccatcgtcgagggccccaactttgagttctccacggagacccatgaggagctgctgtacaacaaggagaagttgctcaacaacggggacaagtgggaggccgagatcgccgccaacatccaggctgactacttgtatcggtgacgccc

SEQ ID NO: 79 - DNA, 444 bp, human
  misc_feature (368)..(368): n is a, c, g, or t
gaagtggagtctcatgacctctcagaataaggtgtcacctccctgaaattgcatatatgtatatagacatgcacacgtgtgcatttgtttgtatacatatatttgtccttcgtatagcaagttttttgctcatcagcagagagcaacagatgttttattgagtgaagccttaaaaagcacacaccacacacagctaactgccaaaatacattgaccgtagtagctgttcaactcctagtacttagaaatacacgtatggttaatgttcagtccaacaaaccacacacagtaaatgtttattaatagtcatggttcgtattttaggtgactgaaattgcaacagtgatcataatgaggtttgttaaaangatagctatattcaaaatgtctatatgtttatttggacttttgaggttaaagacagtcatataaacgtcctgtttc

SEQ ID NO: 80 - DNA, 444 bp, human
gggaggccttatagatgtgcttgggggaaaatgaaggggagaaagtagccatacaggagttcaaagaattccatgcccttcagattagcccaattaccagaaacatcatgaaagatattttaaaaactaattatttactacagtgtatttcacttgtcttgtgtgtctgaacacacagaagctaattagcaagtttttaagaagtatttaaaaatcttactaggattgacattttttctgaattctgtataaatagcttatagtgagaagtactgtgctcaaattttacatttttttcctttgcaaattctgtaatttcactcaacgattaagtctaccaaagaacacactgcatgtaaaagatgtattacaatctcaaagccagtaaaagaaatcttgcttcactgttcacctgctacaagtaagagtttggtgctggtagaa

SEQ ID NO: 81 - DNA, 435 bp, human
cacgctggctgctaagggcgacttggtgttcaccgccatcttcattggggctgtggcggccatgactggctactggttgtcagagcgcagtgaccgtgtgctggagggcttcatctagggcagataatcgcggccaccacctgtaggacctcctcccacccacgctgcccccagagcttgggctgccctcctgctggacactcaggacagcttggtttatttttgagagtggggtaagcacccctacctgccttacagagcagcccaggtacccaggcccgggcagacaaggcccctggggtaaaaagtagccctgaaggtggataccatgagctcttcacctggcggggactggcaggcttcacaatgtgtgaatttcaaaagtttttccttaatggtggctgctagagctttggcccctgcttaggattaggt

SEQ ID NO: 82 - DNA, 319 bp, human
gagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctttatctgaccaaaccgctctacacgtcagcaccatctctgcagcatctctgtaggctcaccattaacaaatgtaccggtgccatctg

SEQ ID NO: 83 - DNA, 533 bp, human
  misc_feature (36)..(36): n is a, c, g, or t
tatgcacaccatcaaatagtttctgtactttttatngggtaaaaatggaattgaacagcaacctcaacataagattttttttctagtagcctcccactgattaaagaagcaagtttgaggtttcatccttcaaaagggggttccgagagagcaccgtagggcttttctcaaatagaaaagccagattttgaaaaaattttaaagataaaataggacatattttgcagatatatatatatatatanacannaacacatctccaggtatagagaaccatccagatgttcacttttgaaaatatctaatgatgcaaagttttattcttgaacttggacactgatgccatcaaacaattaacaaatatatttaagtactaaaggtgatttttttttnaaagactttttcaaattgtcaaatgatttaatgcagatgaacatatttctattttaagtaacgggaatctgtaagaatgtttgcttgagatatggttaacttttttcttttgttggttttgacttagatggacaccatga

SEQ ID NO: 84 - DNA, 479 bp, human
gattcgacgcttggaacttgagaggattcaggcagaagaaagtgtgaaaaccttgtctagagaaacaattgaatataagaaagtactggatgaacagatacaagaaagggagaattcaaagaatgaggaatcaaagcacaatcaagaactgacatctcagttgttagctgcagaaaataaatgcaatctattagaaaaacaattggaatacatgcgaaatatgataaagcatgccgaaatggagaggacatctgtcttagagaaacaagtttccctagaaagagaacgacaacatgatcaaacacatgttcagagccaacttgaaaaattggatcttcttgaacaggagtataacaaacttaccacaatgcaggcccttgcagaaaaaaaaatgcaagagttggaagcaaaactccatgaagaagaacaggaaaggaaacgcatgcaagctaaggcagctgagttgcagactggtctag

SEQ ID NO: 85 - DNA, 325 bp, human
actagtacttagttgccacactcatgcttacatagaaagagagcccaagaatattagatttcctcatgatacaagatactacagtaacaggctttaatttaggatccttaagattttggggtattatttgtgactctcctgaaattgtaaacttgtgcttctgtgtccagttttctaatgagtaggttcgtagcttgattgaattaataattgtgagcccatagacacaagggaagtgagaaacagtgctctggtgacatgataaatatatgtgtcaaccaccatttcagctattaaaaactcctgttatctccttgtttgaatt

SEQ ID NO: 86 - DNA, 442 bp, human
ttctccgatgtctaactctttgtgccttgagctcctttatgtttgggtggcatgttcatgaaaaagccatacttctagcaattctcccaatgagccttttgtctgtgggaaaagcaggagacgcttcgatttttctgattctgaccacaacaggacattattccctctttcctctgctcttcactgcaccagaacttcccattaaaatcttactcatgttactattcaccatatatagtatttcgtcactgaagactttattcagaaaagaaaaacctctttttaattggatggaaactttctacctgcttggcctggggcctctggaagtctgctgtgaatttgtattccctttcacctcctggaaggtgaagtaccccttcatccctttgttactaacctcagtgtattgtgcagtaggcgtcacatatgcttggttcaa

SEQ ID NO: 87 - DNA, 555 bp, human
gactcctaaccaaccagatggactgccagacaggcagacatcagtccacatggaatcctgacatcccagccagccggccagactctcatcttgatgtcttgatggatggaccccagctagtcagacatgatcctccagattgacagacaagtcccccaaatgagtacacatctccagctattcagacagatggagccccagcaaatcaggacctatctaggcagaccccagccagacccccgccagacagactcccaaccagactgaccccttgctgttcacacagcctgccgagtagctgggactacaggtctaattttttttttttttaagaaatgagtttttgccatgttgcccagactggtcttgaactcccaacctcaagcaatcctcctgcctcagcctcccaaagtgctgagattacaggtgtgagccaccaggctcagccccctaagatttgaaacactttaaatggcccatggtagggttcctgctaggataaaacattaagtggctgttaaaagaaataaaaggaggacacgtctctgtgcaaaa

SEQ ID NO: 88 - DNA, 547 bp, human
agaaggagaccctgcagtacttccagaccctcaaggccgtggaccccatgcgggcaacgtatctggatgacctgcgcagcaagttcttgctggagaatagcgtgctcaagatggagtatgccgaggtgcgtgtgctgcacctggctcacaaggatctgacagtgctctgccatctggaacagctgctcttggtcacccatcttgacttgtcacacaatcgcctccgaaccctgccacctgcactggctgccctgcgctgccttgaggtgctgcaggccagtgataatgcaatagagtccctggacggcgtcaccaacctaccccggctgcaggagctgctactgtgcaacaaccgcctccagcagcctgcagtgctccagcctcttgcctcctgccccaggctggtcctcctcaacctgcagggtaacccgctgtgccaagcggtgggcatcttggagcaactggctgaactgctgccttcagttagcagcgtcctcacctaagaggccctgccccctacccttgccctttaacttattgggactga

SEQ ID NO: 89 - DNA, 460 bp, human
gggatactgagggcagtcctgtggctgaggggcacagattgaactgctgaactagttggaggtctagatgaggtgctttacgcatcagctgccttagacagcttctagaaaggagcgagcgctacttcttaagtacttaagtgacatttagataatttatagtaaaactgaaattattattagccaatgcattggtgcatagaatttactagggctacttctggaagcccccaatagaatagcatttccatgtgcattaaatactttgccagcactgcctttgccagcatcctaaatctggagttttaccaagaaggaaactgtatctttaggttaatccaagctatgcatttcatatagctttttcatttaaaacaaggcaaagaaacaaattcctatgaccaaattgcttgcctacagttccctgcagtaattgtatgatctcacccagtgtgcaatt

SEQ ID NO: 90 - DNA, 485 bp, human
  misc_feature (53)..(53): n is a, c, g, or t
tttgttccacgtcaagttctggccttctgatgcaaatgcaaaggaacttagtntgttatgaacccaggttgatgacagaccagtccttgtggaataagattccctttaaaaactctttagccagtcgtgacatcaaccctagacctgtctgccttggcatttgctgtcaanatntgctgggctatgtaggcaggttaatcctccacttctcatgtggttgaaccagtgtgttttttggtaaaatggtgattgtagataagattagttccctgatcccctgccccctgtcccctgcctcttttcccaattcccttccttatgctggacttttaaagcttaaaaaaaatccgattgaatataaatgcctaatttcattcttttgtgaaatggttgcttcctcctgattccctaattgtgctgtgttcgtgtcttgcactggaattcaacattcccttctccttttgtactgtgttgtgcttgctgtc

SEQ ID NO: 91 - DNA, 435 bp, human
caacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgattctcttcagtgaattttgataatggcttccagactctttgcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcact

SEQ ID NO: 92 - DNA, 394 bp, human
  misc_feature (284)..(284): n is a, c, g, or t
tccccaaaatgattccaatagacaacaaagttgaggaaccactggcacatcccaagctaagatacaaggttaaatggcctttttaagtatgtcatactggatctttaaataaagcaaggcttttgttacactttgtcatgttattaaaagcagacctttgggctgtttaaccgtgtaacaaaaatgccacgtgaaaaataaaaatttttattgtatagcaattctctataaatagtagataatctaagtccttattttctgatggctcttgttccactattaanattgtttttaatttttaaaatcctatcagcagcctcctaattagcagtgttaggaatttgccttatgttttccatctcatctcctgaacctgttattctgagaactcata

SEQ ID NO: 93 - DNA, 416 bp, human
gattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaagggagccggtccttgct

SEQ ID NO: 94 - DNA, 437 bp, human
tggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaacccaagcctatttcacagcaaggaaattcaccttcaaaagcactgattacccaatgcacctccctccccagctcgagatcattcttcaattaggacacaaaccagacaggtttaatagcgaatctaattttgaattctgaccatggatacccatc

SEQ ID NO: 95 - DNA, 466 bp, human
tgccagtatgacccacttggactcaccccctcttggggagggagttctgggcctggccaggtctgagatgaggccatgcccctggctgctcttatggagccgagatccagtcagggtgggggcgctggagccgtgggagcccggccagggcagggggctcgtcgctgtggcaccccctctctgccagcaccaagagattatttaatgggctatttaattaaggggtaggaaggtgctgtgggctggtcccacacatccaggaaagaggccagtagagaattctgcccactttttataaaaacttacagcgatggccccaccaaggcctagacacggcactggcctcccaggagggcaggggcattgggaatgggtgggtgccctccagagaggggctgctacctcccagcaggcatgggaagagcactggtgtgggggttccaccgagaaggggacctcatctaga

SEQ ID NO: 96 - DNA, 443 bp, human
aggaatctcagtcacaccaaaccaaccttttcatttcctgctctctcccctcttttgtgaagaaagcgggtccagatgtgattcaaacaactgtacggagtggcatattagaattgccctaaactgaactgcaaataattatgtgtgtatgtatatgtgtgggaaagagaatgtactgtatatgtgtatgttatacagacatatacacatacatacattgacccacaggacattgtaaaatattatcacatgacatcttaagtagaaataagtagggacttttattccatcctttttttcacgtttacattttaattattacaagttgctcctgccccctccctgaactattttgtgctgtgtatatcactgctttatataagttattttttaaggtgaactcagatgttatggttttgtatatgtctgcaatcatggatagg

SEQ ID NO: 97 - DNA, 460 bp, human
  misc_feature (57)..(59): n is a, c, g, or t
gaagctgaactgggccttcaatatgtatgacctggatggtgatggcaagatcacccnnntggagatgctggagatcatcgaggctatctacaaaatggtaggcactgtgatcatgatgaaaatgaatgaggatggcctgacgcctgagcagcgagtagacaagattttcagcaagatggatangaacaaagatgaccagattacactgggtgaattcagagaagctgcaaagagcgacccttccattgtattacttctccagtgcgacatccagaaatgagctgatgtcaatgctatgggctncncccaagtctcnatgttccattcagtctgcagctattcacacacacacacacacacacacacacacacacacacacacacacacncaaatattgcttggnctacctataaatggacttgcttcttgtgtttgaaacactcgtgtgcatgagaatgt

SEQ ID NO: 98 - DNA, 412 bp, human
  misc_feature (258)..(258): n is a, c, g, or t
tcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaaccntcatgctggacaaangatggccacatcaagatcactgactttggcctctgcaaagnagggncatcangtgacggggccaccatgaaaaccttctgtgggaccccnggagtacctggcgcctgaggtgctggaggacaatgactatggccgggcc

SEQ ID NO: 99 - DNA, 488 bp, human
ttccccatctgggccttcataaaatgcaggggaagccagactggtctcaggagcgctaaagcccttccgtggggggtcgtctttctgggactagccctgctgtttaggacctgggaccacaatggggtacctgccgagggggtccccaagagatccaggctgtcatgtgatttatggtggcatgtgttgtgtatttgttggctacttgtgtcttgaaatctagaattatttcacgcagaattgtcactgtttgtcaggaagagaaaatgggctagtggaagcccagtcttgagttcttgtcttgttaccatttaaaattgacatttaattttcaaatcactgttggtgcctaatcacttaagttattaatttattctgttgtattcttttttttaaattgtaacatatttatccggtgggtgggacaggagtgtgttcaagtgggtcatgtttttgctgtggtgacacatggtacaggcttggagctt

SEQ ID NO: 100 - DNA, 558 bp, human
tttttgcctctgtgagttcatctgatgattgagcagtagcatttgccttttgggttttttgtttgttattatagaagagatgacttctgctgattttgctttagaatggttaccttagaagaatttgggtggctcatgttgaatttcacttctgcaatagctttcattttctcataggctttataagagatgggttcagtggtatgagcagaggaagagatcccagatagtagccagttaaccaagactcattcatatagcacgtagtttatgttcctgaggcagcacttttagatcctttgtgagcaagttctatttgttcattgcttgccagagatgaacacagaatgttctgtttcattttacaagaactatcctgagtttctgtggatggaaacattacatgtaatgcagatatagtgaacactggaaagatttattaaagaattatatttgtgtatactttataaattagtccctcattagatttttttttcttaagcataagactgaacttaaatgtgttaattttagtagaatcaggcactgctcgcagaa

SEQ ID NO: 101 - DNA, 444 bp, human
gagccctgattgatatgtatatagaaggtatagcagatttgggtgaaatgatcctccttctgcccgtatgtccacctgaggaaaaagatgccaagcttgccttgatcaaggagaaaataaaaaatcgctacttccctgcctttgaaaaagtcttaaagagccatggacaagactaccttgttggcaacaagctgagccgggctgacattcatctggtggaacttctctactacgtcgaggagcttgactccagtcttatctccagcttccctctgctgaaggccctgaaaaccagaatcagcaacctgcccacagtgaagaagtttctacagcctggcagcccaaggaagcctcccatggatgagaaatctttagaagaagcaaggaagattttcaggttttaataacgcagtcatggaggccaagaacttgcaataccaatgt

SEQ ID NO: 102 - DNA, 372 bp, human
  misc_feature (57)..(57): n is a, c, g, or t
cagccatgtccatgggcctggagatcacgggcaccgcgctggccgtgctgggctggnctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtg

SEQ ID NO: 103 - DNA, 471 bp, human
gtggaccgcgtggactatgacagacagtccgggagtgcagtcatcacgtttgtggagattggagtggctgacaagattttgaaaaagaaagaataccctctttatataaatcaaacctgccatagagttactgtttctccatacacagaaatacacttgaaaaagtatcagatattttcaggaacatctaagaggacagtgcttctgacaggaatggaaggcattcaaatggatgaagaaattgtggaggatttaattaacattcactttcaacgggcaaagaatggaggtggagaagtagatgtggtcaagtgttctctaggtcaacctcacatagcatactttgaagaatagacttaacagaatcatgaaaactatagctttttaacccggattactgtaaatgtttgacaagaatgaatatgcttttccttaaaaaatgaaaactttaatttttaccatccatttatg

SEQ ID NO: 104 - DNA, 537 bp, human
atttacctggtatctacatccttacctgcattggaaaatgtttgctacctcacaacaaccatttgcctcctttaagaacactgatgggctgcactttttggatagaaatagaatttgatttcagaatgtatgcttggtgagtctcagtgcccaggaacacttttggaataatttatcagacattgaacttctgtgattaatcgcttttatagatttactcagtctttaaaattcgtctctgatttgccagagaaaaacggtggtagccatggaaatcgggagtgaaggagcactgcttcattgtggctcagcccttcctaggggcctctgccctttgatgtccttgagctactcttcagctctggaagttgtggacaaaccgtaggaatgtatgtgtgcgtgtggtggagtgattgtctgtgaatgacaggccctggctattgattgatgttgcatcaatttagcaaattcatttcctcattcttgatggcctgaatatatgtctgcacttttaatgctcctcttaaccagttgtaa

SEQ ID NO: 105 - DNA, 390 bp, human
gaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtggg

SEQ ID NO: 106 - DNA, 515 bp, human
  misc_feature (89)..(89): n is a, c, g, or t
aattgccacattttcttatggcattaaaaattttacaaaaacataattttaatggctatattatattccatttaatggatgcaactcangtttatttaaccattcccatngttgttaactatttaggttgtttctaattttcattattataaagttgcagaaatttggtgtacataaaactgtctccatataattgattattaggatatattcccatgaaggattcntttttttaaaaaaatgtgaaatntcatcttgtacttacacctttcatgnaaagggatttcctgcttttgtactgcatgggtggcagttgtgaggaaaagccagtcaaatgacctttttacaaaagaaatgcagtggtcacttcagttgagagtgactttttaatacaacaagatcaactagaagaattcaactgtctcaagaatcaaggtaccccaatatatctcgcaattccaaactttgtttgagggactcgttatccagctcttggtagccacacctgcaatgta

SEQ ID NO: 107 - DNA, 430 bp, human
  misc_feature (43)..(44): n is a, c, g, or t
gggtccatctgtagcaaatgggttgagtgtgtcagtatgtggnntnggttactgtgtattcgccaggaatcaccccgataggctgccaccctattaggtgatacctgtttaatatgttgnccaggtagactagtagttgcatcagtttgctgtaacaagtaaccagtgaggtaacacagtggtgaagcaggtcaggggaggtcaggaggatgtctgagagaaagaagtccgggagatgaatggctgtctaggaaggaggatgtcagtgcacggttagtgtttgagcagagggcagacttgtaaagtacctgtagtgaaaagaatgtggggacccgattagcagaaaggtgtttgcacntactttatacaaaatacagaatactttatattggaagtgaaagaaatgaacgtggacttttacacatgtgca

SEQ ID NO: 108 - DNA, 427 bp, human
aatgagcgccacctttaggcagatcctctgctgccagcgcagtgagaaccccaccggccccacagaaagctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccattta

SEQ ID NO: 109 - DNA, 414 bp, human
ggatcagtgcattactgcctcagacactccagttgtcctcgtcagcgttggagctgacctgcccgtctctgccttagacccaagtcagcttctttgggctgaagagccgacgaacagaaccactctctactcaggcactggcttaagtgttaacagagaaaatgcaaaatgtttcacaaccaaagaggagctggggaagacagtgcctcagctactgactcctgggctgatgggcgaatcttcagaatcctttagtgcctcagaagatgaaggccacagggaataccaagccaatgactctgactcggacgggcctatcttgtacaccgatgatgaggacgaagacgaagatgaggatggcagtggagaaagtgctttggcaagtaaaatacgccggagggatactcttgctat

SEQ ID NO: 110 - DNA, 541 bp, human
cacctagagtctgttgtcatttcactaataggataagacaaattttttgctttgaaaaaatttttctgcatgcccaggtgtctgtctctggctgaggttttgtctattttacagtgtttcaatccagccataaaaattaacttgtgattttttttttcccaaagtcatgcttttccttaattatatttttattttattattttagtgtcttgagaaaaataccaagagatataatgtttcttttaattgtctatgcttaatcatctttaaacactttttaaatttctaaccacaagacctctctataatggtaaatgtaagacatcaccattttatcactcaaagtatgttattgaaagtttctatttggttgataaaaggaacaattttttcccacttttgatgcctgtgatgcaattttttattgcctacaatgagatacacttagtacaaaaaatgaaaatctggtatttcaaaattgcatttcttgtataataggtcagatttattaactactcatactttttctttacactaatcg

SEQ ID NO: 111 - DNA, 306 bp, human
gaggcccagataatgagctgagattcagcatcccctggaggagtcggggtctcagcagaaccccactgtccctccccttggtgctagaggcttgtgtgcacgtgagcgtgcgagtgcacgtccgttatttcagtgacttggtcccgtgggtctagccttcccccctgtggacaaacccccattgtggctcctgccaccctggcagatgactcactgtgggggggtggctgtgggcagtgagcggatgtgactggcgtctgacccgccccttgacccaagcctgtgatgacatggtgctgattctgg

SEQ ID NO: 112 - DNA, 490 bp, human
tctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccaga

SEQ ID NO: 113 - DNA, 491 bp, human
aagcaaatagtgccctcagctactgcagaagaaaagtcccactgaggaaaagaaagtcttgtgatttttaaaggcaagttttcaagtgctctcatagttctatcctctaattccattaaatccatactaggagcgtcagtgagggttttcatagcttttggaaatactttggtctctgaactgtaattagcaagaagtaaaaacagaaacgtcaaacgtcaaatgtttgctttgttacctggaggactaaatgtagatgtctttagtatactttgtatgttcttaaatattggaagataattttgtgaatctgtagattttattttttcagtcttaccttacaaatttcttttctatgaataatagaggaactcacggcactctgccacttgttaatgaaaggaagtgcagaggatttagaaaagtacatgatccccagaccacaacaaaccaaaacataaactcatgtctgtgtcccatggtcatagtca

SEQ ID NO: 114 - DNA, 503 bp, human
ctctcatgcgtgttttaccttttgatgttcagagtggctcactggctgggagtccttacctcggggaggagggggaggttggttccttggggggccaaagaaggcagggaatgcctggagggtaactggggccaccatgaaccccttttctccagaaaagctgcttctccccccatcccgggtcccacccccaaacccccagaggtggcccttgtttacagtgaggactcggccactgtgtctctgtttcctgaaatataaactgtagcgaccccagactgtagagatttttatgtgtttggaacatctgctgtgtggaaaaaaaaaaaaactacaaaaaccctaattttgtacatactgtatttttactattgaactgtattctagtggctgttcatgctccaagactttagttaccgagacatgaatactatccatgtaataagcacttgcctggaataaaatataaaactgaaataaacctgcactgaaacctgagatgg

SEQ ID NO: 115 - DNA, 379 bp, human
cctggtctgccacagatccatgatgtgcagttctctggagcaggcgctggctgtgctggtcactaccttccacaagtactcctgccaagagggcgacaagttcaagctgagtaagggggaaatgaaggaacttctgcacaaggagctgcccagctttgtgggggagaaagtggatgaggaggggctgaagaagctgatgggcagcctggatgagaacagtgaccagcaggtggacttccaggagtatgctgttttcctggcactcatcactgtcatgtgcaatgacttcttccagggctgcccagaccgaccctgaagcagaactcttgacttcctgccatggatctcttgggcccaggactgttgatgcctttgagtt

SEQ ID NO: 116 - DNA, 344 bp, human
  misc_feature (173)..(173): n is a, c, g, or t
gcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtgggctgtgcnaagtgtgcccagggctgcatctgcaaagggacgtcagacaagtgcagctgctgtgcctgatgccaggacagctgtgctctcagatgtaaatagagcaacctatataaacctggatttttttttttttttttttgtacaaccctgacccgtttgctacatctttttttctat

SEQ ID NO: 117 - DNA, 512 bp, human
gagaacgttgccttatactctctacttcagatgatgaacactgtgtactgtgtgtgctttaaagaagttttatttaattgctcccttcttcctttccttgttattcacctccctgatgcctgctttcagttgagggttgggggcaatgatgagcatatgaattttttctcactctagcaattcccttttctaaatgacacagcatttaaactcaaatctggattcagataacagcacctgcacatcctgcacctcctccctctcccttcacctcacccctgcccggcccaagctctacttgtgtacagtgtatattgtataatagacaattgtgtctactacatgtttaaaaacacattgcttgttatttttgaggcttttaaattaaacaaaaatccaactttatttttagttgtaactgcttgaggtatgttttatgaattaagtgacagatttgttatcctttattaacgtactttgttggtcagcactgggctgacaaaaattttttc

SEQ ID NO: 118 - DNA, 396 bp, human
attcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacga

SEQ ID NO: 119 - DNA, 117 bp, human
gatttaagatggataacaaactgcagttaatccctggggtctgtggattccgaatttcaaatcctcccattttgttggtctgttccttgcatgctagtttagagatctttaagcaag

SEQ ID NO: 120 - DNA, 318 bp, human
ttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttggcttaattccaatcattgttaaagtaaaaacaattcaaagaatcacctaattaatttcagtaagatcaagctccatcttatttgtcagtgtagatcaactcatgttaattgatagaataaagccttgtgatcact

SEQ ID NO: 121 - DNA, 503 bp, human
tatcggctacatatgcagtctgtgaattatgtaacatactctatttcttgagggctgcaaattgctaagtgctcaaaatagagtaagttttaaattgaaaattacataagatttaatgcccttcaaatggtttcatttagccttgagaatggttttttgaaacttggccacactaaaatgtttttttttttttacgtagaatgtgggataaacttgatgaactccaagttcacagtgtcatttcttcagaactccccttcattgaatagtgatcatttattaaatgataaattgcactcgctgaaagagcacgtcatgaagcaccatggaatcaaagagaaagatataaattcgttcccacagccttcaagctgcagtgttttagattgcttcaaaaaatgaaaaagttttgcctttttcgatatagtgaccttctttgcatattaaaatgtttaccacaatgtcccatttctagttaagtcttcgcacttgaaagctaacat

SEQ ID NO: 122 - DNA, 506 bp, human
actgcacttcgaatgtgttaccaaggttatggttaccctttgatgctgttcctggaagaaggaggagtggtgacagtctgcaaaatcaatacacaggaacctgaggagaccctggactttgatttctgcagcaccaatgttattaataaaattattctgcagtcagaggggctccgtgaagcattttctgaattggatatgacgagtgaagtcctacaaattaccatgtctcctgacaagccttatttcaggttatctacttttggaaatgcaggaagttcccaccttgactatcccaaagattctgatttgatggaagcatttcattgtaatcagacccaagtcaacagatacaagatttccttactgaaaccctctacaaaggcattagtcctatcttgtaaggtatctattcggacagataacagaggcttcctttcattacagtatatgattagaaatgaagatggacaaatatgttttgtggaatattactgctgccctga

SEQ ID NO: 123 - DNA, 358 bp, human
gtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattg

SEQ ID NO: 124 - DNA, 117 bp, human
gaaaatgtccatttacacgtatttgaatggccttcatatcatccacacatgaatctgcacatctgtaaatctacacacggtgcctttatttccactgtgcaggttcccacttaaaaa

SEQ ID NO: 125 - DNA, 357 bp, human
attaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaa

SEQ ID NO: 126 - DNA, 553 bp, human
tgttcaatataggacaccccagcttgtccttggctcatccaagaactcttctgtatctaaaacaatacatctcaatcttggccaagggaaaatggactgctttgctggattggcactgagcaactttaggaaatgtcggtggagtgttcagcaagatcagacagcagtccaggtcaaaggcaaacacacacgctccagcccaaatcctcctggtggcacatcctaccccagatgctaaagtgattcaaggactccaggacacctcttaagagcctttctaagaacatgataggcttacttctgctccataataaagtgggagaaaaaagccagaatataacttaagactagataactgcgtacatgatggaccattttttttttttttggctgggtagagaaatcatataaaacgcaggctgtttagcatggagatgactctcagaacactgggagggtctggcacttgatgggggttagttgcttggcagcctgcctgccactgagggaagtcccattagagatgtatcaccaccttgtcaccaacaggatg

SEQ ID NO: 127 - DNA, 514 bp, human
gctcttgttttcgatgtgtgtttgtaaagggaaaattagtactctgctcgactcttggtaacatgaaattctgaatgttactttatcatgattgcactgcaacttttttccttaaaataactgcttttgtaagaacggtgatattggagtgattagtataaattcaatggaatttgagaagcaatggcagcgggataatttagagtcactgatattacgagaggggtctttttgtaaacctccttttcaatgtcaaagcaccaatttataaaacgctgcagatgtagaggttatgtgcaactgatctgtccagtttgtgtatgaaatggatttgataaagtttttgctagttatttactacattttgggattaataagtgatttatatgcatatttttctgtaaatctacagttttttgtacaagatattctacaagttatgaagctaagggaagaaaatgccaaagatacctctagttatgttgaacacagccagcacagtttcgacaggtca

SEQ ID NO: 128 - DNA, 465 bp, human
ttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatat

SEQ ID NO: 129 - DNA, 531 bp, human
  misc_feature (280)..(280): n is a, c, g, or t
agagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataangtataatgggaaatgtttggccatgcttggttgttgcagttcagacaaatgtaacacacacacacatacacacacacacacacacanngagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttt

SEQ ID NO: 130 - DNA, 418 bp, human
ggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataa

SEQ ID NO: 131 - DNA, 474 bp, human
cagtgatccattgcacctttattccctcagcatatgcctgaggcgatcttttatgcttttaaaaatgtttcccgttggtctcattggaatctgcctcctaatgattttttttcaggaaaacctgtttggttatcctcattcaaaagcactggacagagaatgttactgtgaatagagcacatattactctttttagcaacctagcatgatgccaacaagactatttttgaaagagcaaaggttcctgtaaatttaattagggctagatttgagctgcttgtaagtcacaggttttccagatgtctgccaacaagaaatgactcatactgtgatgataccttttgctttgccttgtggacaatgtgggtttttgaaatttgcacccttcaaacaatgatttatcagagaaaggggtctgttttcaaaaaagattctgtaatgaattttatgtgtggcatatacttatttcttgag

SEQ ID NO: 132 - DNA, 563 bp, human
gtgcaggcggtgcctttggtgaagctgcaggagacaggggtccctatggcactgacatcggcccaggcggaggctatggggcagcagcagaaggcggcatgtatgctggcaatggcggactattgggagctgactttgctggagatctggattacaatgagctggctgtgagggtgtcagagagcatgcagcgtcagggcctactgcaagggatggcctacactgtccagggcccaccaggccagcctgggccacaggggccacccggcatcagcaaggtcttctctgcctacagcaacgtgactgcggacctcatggacttcttccaaacttatggagccattcaaggaccccctgggcaaaaaggagagatgggcactccaggacccaaaggtgacaggggccctgctgggccaccaggtcatcctgggccacctggccctcgaggacacaagggagaaaaaggagacaaaggtgaccaagtctatgctgggcggagaaggagaagaagtattgctgtcaagccgtgagctagccatggcaggacagctcctggaccaggt

SEQ ID NO: 133 - DNA, 273 bp, human
acagaacaggctcagtcagcatcctcacccagagatggcaacatctattaagaccaatgcaataccttttcatcttcagcaaatgttgtttcatctgtttttgatccttggcattgtcaaaaacttaactgcaggtccagtgtatatttttccttatttttcccttttagctatctgctaaagcgagtaaatgccacaactgtacttttccaaagaaaaagaactattgacaacttatagcctgtcatgcaggtcatgtttcaaatcaaggct

SEQ ID NO: 134 - DNA, 543 bp, human
tggccgagacagagtgccgctatgccacgcagctgcagcagatccaggggctcattggtggcctggaggcccagctgagtgagctccgatgcgagatggaggctcagaaccaggagtacaagatgctgcttgacataaagacacggctggagcaggagatcgctacttaccgcagcctgctcgagggccaggatgccaagatggctggcattggcatcagggaagcctcttcaggaggtggtggtagcagcagcaatttccacatcaatgtagaagagtcagtggatggacaggtggtttcttcccacaagagagaaatctaagtgtctattgcaggagaaacgtcccttgccactccccactctcatcaggccaagtggaggactggccagagggcctgcacatgcaaactccagtccctgccttcagagagctgaaaagggtccctcggtcttttatttcagggctttgcatgcgctctattccccctctgcctctccccaccttctttggagcaaggagatgcagctgtattgtgtaaca

SEQ ID NO: 135 - DNA, 360 bp, human
  misc_feature (250)..(250): n is a, c, g, or t
attttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaantctgagttcctggaatggcacgttgctgccagtgccccagacagttcttttctaccctgcgggcccgcacgttttatgaggttgatatcggtgctatgtgtttggtttat

SEQ ID NO: 136 - DNA, 538 bp, human
  misc_feature (314)..(319): n is a, c, g, or t
gtcaattagagcgatcccaaggcatgggaccaggcctgcttgcctatgtgtgatggcaattggagatctggatttagcactggggtctcagcaccctgcaggtgtctgagactaagtgatctgccctccaggtggcgatcaccttctgctcctaggtacccccactggcaaggccaaggtctcctccacgttttttctgcaattaataatgtcatttaaaaaatgagcaaagccttatccgaatcggatatagcaactaaagtcaatacattttgcaggaggctaagtgtaagagtgtgtgtgtgtgtgtgtgnnnnnncgtgcatgtgtgtgtgtgtgtatgtgtgtgaataagtcgacataaagtctttaattttgagcaccttaccaaacataacaataatccattatccttttggcaacaccacaaagatcgcatctgttaaacaggtacaagttgacatgaggttagtttaattgtacaccatgatattggtggtatttatgctgttaagtccaaacctttatctgtctgtta

SEQ ID NO: 137 - DNA, 521 bp, human
atgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgca

SEQ ID NO: 138 - DNA, 547 bp, human
cttcgttcgcagagcttttcagattgtggaatgttggataaggaattatagacctctagtagctgaaatgcaagaccccaagaggaagttcagatcttaatataaattcactttcatttttgatagctgtcccatctggtcatgtggttggcactagactggtggcaggggcttctagctgactcgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttgggtggataagctatccctgttgccggttcatggattacttctctataaaaaatatatatttaccaaaaaattttgtgacattccttctcccatctcttccttgacatgcattgtaaataggttcttcttgttctgag

SEQ ID NO: 139 - DNA, 483 bp, human
ggtagtactagttcacctaaggccttatcacctcctccttctgtggattcaaattacccaacgagagatagagcatctttcaacagaatggtcatgcatagtgctgcttctccaacacaggcaccaatccttaatccctctatggttacaaatgaaggacttggtcttacaactacagcttctggaacagacatctcttctaatagtctaaaagattgtcttcctaaatcagcacaacttttgaaatctgtttttgtgaaaaatgttggttgggctacacagttaactagtggagctgtgtgggttcagtttaatgatgggtcccagttggttgtgcaggcaggagtgtcttctatcagttatacctcaccaaatggtcaaacaactaggtatggagaaaatgaaaaattaccagactacatcaaacagaaattacagtgtctgtcttccatccttttgatgttttctaatccgactcctaat

SEQ ID NO: 140 - DNA, 373 bp, human
tgcgctggtttgcggctttgggaaataaaataccgttgtatatattctggcaggggtgttctagctttttgaggacagctcctgtatccttctcatccttgtctctccgcttgtcctcttgtgatgttaggacagagtgagagaagtcagctgtcacggggaaggtgagagagaggatgctaagcttcctactcactttctcctagccagcctggactttggagcgtggggtgggtgggacaatggctccccactctaagcactgcctcccctactccccgcatctttggggaatcggttccccatatgtcttccttactagactgtgagctcctcgagggcagggaccgtgccttatgtctgtgtgtgatca

SEQ ID NO: 141 - DNA, 364 bp, human
gccaaaatgatacctggaggcttatctgaggccaaacccgccactccagaaatccaggagattgttgataaggttaaaccacagcttgaagaaaaaacaaatgagacttatggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctga

SEQ ID NO: 142 - DNA, 523 bp, human
aacagagatgtcccccagggagcacatcaagggcaaagagaccaccccctctagcctagcagtgacccagaccatggccaccaaagctcccgagtgtgtggaggacccagatatggcaaaccagaggaagactgccctggagttctgtggagagacttggagctctctctgcacattcttcctcagcatagtgcaggacacgtcatgctaatgaggtcaaaagagaacgggttcctttaagagatgtcatgtcgtaagtccctctgtatactttaaagctctctacagtccccccaaaatatgaacttttgtgcttagtgagtgcaacgaaatatttaaacaagttttgtattttttgcttttgtgttttggaatttgccttatttttcttggatgcgatgttcagaggctgtttcctgcagcatgtatttccatggcccacacagctatgtgtttgagcagcgaagagtctttgagctgaatgagccagagtgataatttcagtgcaacgaactttctgctg

SEQ ID NO: 143 - DNA, 389 bp, human
cttgtggcaccaggcatctgatggtgccaggaaagccactggaattgtcacacggcgagcacagagggccggccaccagtcctcgatgcttctgaaccctgaaccccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgcacgcacgcacacacacacacacacacacacacacacacacacacacacgaaggagagagagagaaaactgatggggaaaacaagctgtgtcttcttaactgcccaagtgaaa

SEQ ID NO: 144 - DNA, 512 bp, human
tcttccgtgccatgtatgactatatggctgctgatgcagatgaggtgtccttcaaggatggagatgccatcataaatgttcaagcaattgatgaaggctggatgtatggcactgtgcagaggactggcaggaccggaatgctcccagccaactacgttgaagctatttaggcatttcaaagcatcacacttgtctgcaggacttacagatcctgcagtcaatgtttcggtttagactctccactgttacctaagttctcaagctgcctatggtttttctgtgtcaatgtgatttatggtagtaccatcctttctcctttgggttttaaaataagttgcagaacagacactttaaaagcttctgcaatattatttctgtgcctagagtctttctccattataaacatgttttaacattatttcttttctaaaacagggattttgaatatgccaaacacattaaaggaaaaatagcagagatgttcaccttttccttgctgattgctaatgctt

SEQ ID NO: 145 - DNA, 530 bp, human
tacagagtattcctctcttcacaccaggaccagccactgttgcagcatgagttcccagcagcagaagcagccctgcatcccaccccctcagcttcagcagcagcaggtgaaacagccttgccagcctccacctcaggaaccatgcatccccaaaaccaaggagccctgccaccccaaggtgcctgagccctgccaccccaaagtgcctgagccctgccagcccaagcttccagagccatgccaccccaaggtgcctgagccctgcccttcaatagtcactccagcaccagcccagcagaagaccaagcagaagtaatgtggtccacagccatgcccttgaggagccggccaccagatgctgaatcccctatcccattctgtgtatgagtcccatttgccttgcaattagcattctgtctcccccaaaaaagaatgtgctatgaagctttctttcctacacactctgagtctctgaatgaagctgaaggtcttagtaccagagctagttttcagctgctcagaattcatct

SEQ ID NO: 146 - DNA, 193 bp, human
gtcccaagtgcaacaaggaggtgtacttcgccgagagggtgacctctctgggcaaggactggcatcggccctgcctgaagtgcgagaaatgtgggaagacgctgacctctgggggccacgctgagcacgaaggcaaaccctactgcaaccacccctgctacgcagccatgtttgggcctaaaggctttgggcg

SEQ ID NO: 147 - DNA, 495 bp, human
gataactccaggtacacacacttcctgacccagcactatgatgccaaaccacagggccgggatgacagatactgtgaaagcatcatgaggagacggggcctgacctcaccctgcaaagacatcaacacatttattcatggcaacaagcgcagcatcaaggccatctgtgaaaacaagaatggaaaccctcacagagaaaacctaagaataagcaagtcttctttccaggtcaccacttgcaagctacatggaggttccccctggcctccatgccagtaccgagccacagcggggttcagaaacgttgttgttgcttgtgaaaatggcttacctgtccacttggatcagtcaattttccgtcgtccgtaaccagcgggcccctggtcaagtgctggctctgctgtccttgccttccatttcccctctgcacccagaacagtggtggcaacattcattgccaagggcccaaagaaagagctacctggaccttttgtt

SEQ ID NO: 148 - DNA, 335 bp, human
gcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgacca

SEQ ID NO: 149 - DNA, 464 bp, human
attggaatctcctgtgcatcacatttcttaaaaagagaaatgcagaaaaccaaagaactaatgacaaaagtgaataaacaaaaactgtttgaaaagagtacaggacttcctcacaaagcatcacgtcatcttgacagctatgaattccttaaagccattttaaactgaggcattaagaagaaatgcactcaccatgagcaccaacttctgcatctgcctgatcatatttaaaggaacagagaaatatttgtaattaatctgcccagtaaataccagctcgtagcagttggcaggtgcatgtctagataaaatttcttgcagctaatttaaactttctacacgcaccagtagataatctcaatgtaaataatacatttcttcttggctctttaatgtaagccaacatggagaggaagatcttgacttatattctgtaccacatacacttctgtggacttttagca

SEQ ID NO: 150 - DNA, 545 bp, human
tgccgtgggttttcaagtttactcatttctatggttgcaaataactctaaaacttattatataaactttcatattataggcagaacacaatggctaaatatctgttgcatgtactttaaagtttattataaaatataaacagatatataaagatgttgactcttacctgtgattttgcatggtcagactcggtgtcaggtacggagaggattctcatgactgtcttacctctactgaatattctagtgagttatatgatttacggagtgattaacagaggtctatataaagttacttttcccctttacttaattatattgtagtgtgcagataacaaaactgctaccttctcatccaagtggtctgtagaattcatgtcccttacagtggtcatttaaagtcaatatttatttatgtatgtaataaaaaaagttggatttttgtgtatgtctgtcacattatttagagagaagtaatcttgtaaaaatgttttgtaaaaaacaaaaaagtattgtaaatagtcttgatattctgtgactcattat

SEQ ID NO: 151 - DNA, 243 bp, human
gatctgcaggtgtctgggaattagcctggaggaactccgaacccaaatcctgagtccaaacactcaagatgtgctcatcttcaaactctatcagcgggcaaagcatgtgtacagcgaggctgcgcgagtgctccagtttaagaagatatgtgaagaagcacctgaaaacatggtccagctgctgggagagttgatgaaccagagccacatgagctgccgggacatgtatgagtgcagctgccc

SEQ ID NO: 152 - DNA, 388 bp, human
gcacccagccttttaccatgtgggtttctttagtgtcttaaaagcgtccataagccaccattctgtggaaccaaggccccctccacgcaaacaccctccctcctggggacctctggagcctcagccagaagtaccattaggtttaattttaatttgttttgctggagaaacatcaggtttgtaggagactgagttgttagcaggtgtgcttagctcttgatagtgaacgtgtaccttgggaactggctcacccacctgctaatagcaccatcgtcactattaagcagacatttcagttggtagaatccatgtagaagtcatggacttttctgggaaatgacttttctgggaaatgacagtttctttgacatattttctttgcccactt

SEQ ID NO: 153 - DNA, 472 bp, human
gccagtgtcacgtacagcatctgtggttccagtctgtgcttgacatgctccgccacttccacacacaccccatcccactggagtcagggggctcggccgacatcacccttcgcagctatgtgcgggcccaggaccccccaccagagccgggccccacgccccctgccgcgcccgcgtccccggcctgctggagcgactcgcccggccagcactacttctccagcctcgccgcggccgcctgcccgcctgcctcgccctccgacgccgccggcgcctcctcgtcttccgcctcgtcgtcctctgccgcgtcggggcccgcccccccgcgccccgtcgagggccagctcagcgcgcggagccgcagcaacagcgccgagcgcctgctggaggccgtggccgccaccgccgccgaggagcccccggaggccgcgcccggccgcgcgcgcgccgtggagaaccagtactccttcta

SEQ ID NO: 154 - DNA, 527 bp, human
tgttactaacagccctttgaatttaacaaaaactgggaatccattaggaaacggattgcatcatacctgaacataagctggactgctgaaattgtatttttagctaatgaaaaagtgtttggactagtactctaaaaatgttctaatgataaagttttgagtcaaaatagaaaagaaaaaaatctgcattccaggccgaattttgtatatttttattgcatttaaaattgctattctgtaatattgggaaatcaagtggcttatcatgtatatcgtgtacttaaaatgtattcacaaactactgttgtatttgtataaaatatagacaaagatcatattttttgtgtgtgtataagctctgtaaaatagcaatcacattatgaagctgcagtgatactacattttaaacattcacatccaaagaagcagactatttattgtccatataccagatttaaaatattaatttgctgctaattaaataatagtactgcagcttcttgtggcctacagtgttatgtttgctg

SEQ ID NO: 155 - DNA, 515 bp, human
tattgccaatgacttgttccttcaagaacagaaaacccttggcgcagatgataccatgttggcaaaggatcccgcatctggcatctgtactcttctgtatgacagtgcacccagtggcaggtttggcaccatgacctacctctccaaggcagccgccacctacgtgcaggagttcctgccccacagcatctgtgccatgcaatgagggctttggttcctggcttctgggagccttttgacagctggtccctgcctcggttgattgtgcatggaactaaaatgttattgcctaatcactccaaccctgcccctttctgtcccatccttcccaagaagagagaactttttcgataaactaactactgtagaagaagtgaacacttacctggaggctcaccttgcagaaccagtgacaatcttatgagtataatgaacactcagccaggcctgtcatgattggctttatttctttcatcattcataaaagtttgcatgtgtttttattctctagatct

SEQ ID NO: 156 - DNA, 493 bp, human
  misc_feature (87)..(87): n is a, c, g, or t
cactgctagcagggcttcaaccaggaagggatcaacccaggaagggatgatcaggagaggcttccctgaggacataatgtgtaaganaggtgagaagtgctcccaagcagacacaacagcagcacagaggtctggaggccacacaaaaagtgatgctcgccctgggctagcctcagcagacctaaggcatctctactccctccagaggagccgcccagattcctgcagtggagaggaggtcttccagcagcagcaggtctggagggctgagaatgaacctgactagaggttctggagatacccagaggtcccccaggtcatcacttggctcagtggaagccctctttccccaaatcctactccctcagcctcaggcagtggtgctcccatcttcctccccacaactgtgctcaggctggtgccagcctttcagaccctgctcccagggacttgggtggatgcgctgatagaacatcctcaagacagtttcctt

SEQ ID NO: 157 - DNA, 469 bp, human
agcctatccttaataaatcctccactctctggaaggagactgaggggctttgtaaaacattagtcagttgctcatttttatgggattgcttagctgggctgtaaagatgaaggcatcaaataaactcaaagtatttttaaatttttttgataatagagaaacttcgctaaccaactgttctttcttgagtgtatagccccatcttgtggtaacttgctgcttctgcacttcatatccatatttcctattgttcactttattctgtagagcagcctgccaagaattttatttctgctgttttttttgctgctaaagaaaggaactaagtcaggatgttaacagaaaagtccacataaccctagaattcttagtcaaggaataattcaagtcagcctagagaccatgttgactttcctcatgtgtttccttatgactcagtaagttggcaaggtcctgactttagtcttaa

SEQ ID NO: 158 - DNA, 545 bp, human
ccatcccatagaagtccagcagacaggatttgttaagtgccagactttgtcaggaagtcaaggagcttctgctttgtccgcctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtac

SEQ ID NO: 159 - DNA, 444 bp, human
gtctttcttacctgattgatattacattcacctttgattgttttttaaaagtttatttttacagaatatatttagtacctttcttaaggagtaactgaattgaatcaaccagtttgcatttaaataaaagaacaggctcagtggtcttcctgtagaatggtttacatgcctgcatgtgcagtagttgtgtctggaatcctagaattggcactttctgcctccttgctctaaatgtcacaaaaaattatacttccttaaagtaaatgtaatgatttcttcttttcctattgaccagtacagatagatatgttgtgtttgcttcatttttaatgatgacttcaagattgatgatgtgatccaataactgtggaggtagctttaacttggttctgtgtaaatagtatgtattttattataatatttctcattttaagatgcttggtt

SEQ ID NO: 160 - DNA, 355 bp, human
tcaaccagcgtgagaagcccctggccctctacatgttctccagcaacgacaaggtgattaagaagatgattgcagagacatccagtggtggggtggcggccaacgatgtcatcgtccacatcaccttgcactctctgcccttcgggggcgtggggaacagcggcatgggatcctaccatggcaagaagagcttcgagactttctctcaccgccgctcttgcctggtgaggcctctgatgaatgatgaaggcctgaaggtcagataccccccgagcccggccaagatgacccagcactgaggaggggttgctccgcctggcctggccatactgtgtcccatcggagtgcggaccac

SEQ ID NO: 161 - DNA, 565 bp, human
tatgaaacccgctacatctatggcccaatagaatcaacaatttacccgatatcaggttcttctttagactgggcttatgacctgggcatcaaacacacatttgcctttgagctccgagataaaggcaaatttggttttctccttccagaatcccggataaagccaacgtgcagagagaccatgctagctgtcaaatttattgccaagtatatcctcaagcatacttcctaaagaactgccctctgtttggaataagccaattaatccttttttgtgcctttcatcagaaagtcaatcttcagttatccccaaatgcagcttctatttcacctgaatccttctcttgctcatttaagtcccatgttactgctgtttgcttttacttactttcagtagcaccataacgaagtagctttaagtgaaaccttttaactacctttctttgctccaagtgaagtttggacccagcagaaagcattattttgaaaggtgatatacagtggggcacagaaaacaaatgaaaaccctcagtttctcacagattttcaccatgtggcttcatcaa

SEQ ID NO: 162 - DNA, 462 bp, human
gtcatgaggcagctttcatcacacccttttaacatttatctaaaagaatttaaattctttttcaaaaattacactacaagtttataagcccaaatggctctgtgaaatcagaagtgcaaaggtgtgcaaacttgtatctgaagacctaccagggacaagcaggtaagagctgatgtgagtgtgtgtgatgggatctgtaaggaactggaacacacatgtcctatccaaaggaatcagctgcagctgcttgttgtcaagtataaagtcaggacctggcttggctttaaccgtttttcaagaaaactggaaatctggattttcagcgaacatgcctgattttaaaaggttgactcaagtttttacaaaatactatgtgggacacctcaaatacatacctactgactgatgacaaacccaggagtttgtgtgtcttttataaaaagtttgccctggatgtcatat

SEQ ID NO: 163 - DNA, 498 bp, human
cactggattttattggagccccatctccccagcgggcaggcgggcggagcctgtatatatgtatatactcagtgcctcagttcagcttcctccacctcgcttccactgcacaggcccaggaaggagaaaggccaagccaaagtgggccccaccctgcccccgtcgtgctccatccttccctgccggggcctgctggcccctgtaaggtcccgcccccaaagaccctggggccagcggggccgaaagcggagttgggtttgcttattttgctcattggattcaagttcttttgcatagtttttttctaacccctgttggagtccaggggctggagaaaaggacagatttatgcagctattttcatacattccctgttcagagtggggtaggggttttccgccgttacccgatccaatccatcccccaccctttgaggggtgagtgtgtctttgcatgtttcctttgctgtggtgggagatagtttgactgaacccccac

SEQ ID NO: 164 - DNA, 298 bp, human
aaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacattgaactgagaattctacaaactataaaacattaatttttgaaggtaatttagttttgtggctgggcattcag

SEQ ID NO: 165 - DNA, 477 bp, human
attgtggtgagcgatcctgactccacagatgcttcaagcattgaagacaatgaggacatttgtaacaccacctccttggagaattgcacagcaaaatgagcgggggtgtttgtgcctgtttctcttatcctttcccaacattaggttaacacagctttataaacctcagtgggttcgttaaaatcatttaattctcagggtgtacctttccagccatagttggacattcattgctgaattctgaaatgatagaattgtctttatttttctctgtgaggtcaattaaatgccttgttctgaaatttattttttacaagagagagttgtgatatagtttggaatataagataaatggtattgggtggggtttgtggctacagcttatgcatcattctgtgtttgtcatttactcacattgagctaactttaaattactgacaagtagaatcaaaggtgcagctgactgagacgacatgc

SEQ ID NO: 166 - DNA, 474 bp, human
ggttcctagatgtgagggcagcccctttgttggctgatgacaacaaattacgtggcttacccctgacctatgtcatcacctgtcaatatgatctcttaagagatgatggactcatgtatgtcacccgacttcgcaacactggggttcaggtgactcataaccatgttgaggatggattccatggagcattttcatttctgggacttaaaattagtcacagacttataaatcagtatattgagtggctaaaggaaaatctatagtaaaacatgtagctataacatattttaaaaataaaatctgaaaacctcagaaaatttcgattagaaattggtctttcttagaatggtctagttaagttccacatgtagcataattcttaaataggcacttttctgttttttttttcttactgtgggatttcatttcaattttctacattgtctatctgctttttcggagattttccttctt

SEQ ID NO: 167 - DNA, 438 bp, human
  misc_feature (195)..(195): n is a, c, g, or t
atcactattttgaagcacagctttacagatgagtatctatgatacatatgtataataaattttgattgggtattaaaagtattagaaggtggttataattgcagagtattccatgaatagtacactgacacaggggttttactttgaggaccagtgtagtcaagggaaaacatgagttaaaaagaaaagcaggcnatattgcagtcttgattctgccacttacaggatagataacgcctgaactttaatgacaagatgatccaaccataaaggtgctctgtgcttcacagtgaatcttttccccatgcaggagtgtgctcccctacaaacgttaagactgatcatttcaaaaatctattagctatatcaaaagccttacattttaatataggttgaaccaaaatttcaattccagtaacttctattgtaaccattatt

SEQ ID NO: 168 - DNA, 388 bp, human
ccccaagactttactagtgccgataaactttctcaaagagcaaccagtatcacttccctgtttataaaacctctaaccatctctttgttctttgaacatgctgaaaaccacctggtctgcatgtatgcccgaatttgtaattcttttctctcaaatgaaaatttaattttagggattcatttctatattttcacatatgtagtattattatttccttatatgtgtaaggtgaaatttatggtatttgagtgtgcaagaaaatatatttttaaagctttcatttttcccccagtgaatgatttagaattttttatgtaaatatacagaatgttttttcttacttttataaggaagcagctgtctaaaatgcagtggggtttgttttgca

SEQ ID NO: 169 - DNA, 385 bp, human
aaacagttgctcacctacagacagtgcaacataaattagcagaattaaaaacacatatatgtgtaacccgagcatttgtggacaactgtctccagctgcatgaagcgaaacgtttggactccgccactgcttgcatggcgaaatattgggcatctgagttacaaaatagtgtagcttacgactgtgtacagctccatggaggttggggatacatgtgggagtacccaattgcaaaagcttatgtggatgccagagttcagccaatctatggtggtacaaatgaaataatgaaggagctgattgcaagagagattgtctttgacaagtagacatctgcccacatcctggagtcctattacagctaatctcgttttaaatctgctca

SEQ ID NO: 170 - DNA, 268 bp, human
tatttgaatatgtatcgtgccccctccggagtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcattgatccttaaccaataatggtcagtcctcatctctgtcctgacttcataggt

SEQ ID NO: 171 - DNA, 479 bp, human
gaccttcccgctgcggacagggaagaggcaacctggccagcggcggcccgctctgggggccggggtacgcgaccacccaaccgagcagaggctttgggtacagaccccccagctactcgacagcctacctgcctggcagctatggctcttcccactgcaaactggaagccccctcaccgtgctccctccctcagagtgaccctaggctccagggggaactgctgcccacctatacccactacctgccccctggctctcccactccatacaaccctccccttgctggtgcccccatgcccctaacccacctctaaccctcatggacgcagacctcacgggacgggcctcatcctccttttttaatccagcagcatcccctaccccaggctgtcaaccctttctcctgttggactacagttcagaggcagcctgcagtcctcccatgatagccagggagagccgcacaacatacaattata

SEQ ID NO: 172 - DNA, 552 bp, human
  misc_feature (487)..(487): n is a, c, g, or t
gatcaacaattctttttgggggtagattagaaaacccttacactttggctatgaacaaataataaaaattattctttaaagtaatgtctttaaaggcaaagggaagggtaaagtcggaccagtgtcaaggaaagtttgttttattgaggtggaaaaatagccccaagcagagaaaaggagggtaggtctgcattataactgtctgtgtgaagcaatcatttagttactttgattaatttttcttttctccttatctgtgcagtacaggttgcttgtttacatgaagatcatgctatattttatatatgtagcccctaatgcaaagctctttacctcttgctattttgttatatatatttcagatgacatctccctgctaatgctcagagatcttttttcactgtaagaggtaacctttaacaatatgggtattacctttgtctcttcataccggttttatgacaaaggtctattgaatttatttgtntgtaagtttctactcccatcaaagcagctttctaagtttattgccttgggttattatggaatgat

SEQ ID NO: 173 - DNA, 173 bp, human
gcccctaatgcaaagctctttacctcttgctattttgttatatatatttcagatgacatctccctgctaatgctcagagatcttttttcactgtaagaggtaacctttaacaatatgggtattacctttgtctcttcataccggttttatgacaaaggtctattgaatttatt

SEQ ID NO: 174 - DNA, 451 bp, human
gaattgaccctatcttggacagcacctggagaagactttgatcagggccaggctacaagctatgaaataagaatgagtaaaagtctacagaatatccaagatgactttaacaatgctattttagtaaatacatcaaagcgaaatcctcagcaagctggcatcagggagatatttacgttctcaccccagatttccacgaatggacctgaacatcagccaaatggagaaacacatgaaagccacagaatttatgttgcaatacgagcaatggataggaactccttacagtctgctgtatctaacattgcccaggcgcctctgtttattccccccaattctgatcctgtacctgccagagattatcttatattgaaaggagttttaacagcaatgggtttgataggaatcatttgccttattatagttgtgacacatcatactttaagcagga

SEQ ID NO: 175 - DNA, 497 bp, human
cctgtcctccagcaccatagagaggcgctagagcccattcctctttctccaccgtcacccaacatcaatcctttaccactctaccaaataatttcatattcaagcttcagaagctagtgaccatcttcataatttgctggagaagtgtatttcttccccttactctcacacctgggcaaactttcttcagtgtttttcatttcttacgttctttcacttcaagggagaatatagaagcatttgatattatctacaaacactgcagaacagcatcatgtcataaacgattctgagccattcacactttttatttaattaaatgtatttaattaaatctcaaatttattttaatgtaaagaacttaaattatgttttaaacacatgccttaaatttgtttaattaaatttaactctggtttctaccagctcatacaaaataaatggtttctgaaaatgtttaagtattaacttacaaggatataggtttttctcatgta

SEQ ID NO: 176 - DNA, 311 bp, human
ctttttgtaactaccctctcaaatggacaataccagaagtgaattatccctgctggctttcttttctctatgaaaagcaactgagtacaattgttatgatctactcatttgctgacacatcagttatatcttgtggcatatccattgtggaaactggatgaacaggatgtataatatgcaatcttacttctatatcattaggaaaacatcttagttgatgctacaaaacaccttgtcaacctcttcctgtcttaccaaacagtgggagggaattcctagctgtaaatataaattttgcccttccatttcta

SEQ ID NO: 177 - DNA, 386 bp, human
cgccctggcacggtgctgagaattcgcggcttggttcctcccaatgccagcaggttccatgtaaacctgctgtgcggggaggagcagggctccgatgccgccctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctgagcagaagcccaggcggc

SEQ ID NO: 178 - DNA, 244 bp, human
gccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctgatgtcgggacagccctgctgtcagatgaaaacagaatgacacgtaaaatccgaggttttttttttctacaactccgactcatttgctacattcctttttttctg

SEQ ID NO: 179 - DNA, 557 bp, human
ttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcaatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactga

SEQ ID NO: 180 - DNA, 397 bp, human
gtgaacatttcaaccagccttatagctgttctcatcatcaccttctgcattgtgaccgtgcttggaagggaggctctcaccaaaggggcgctgtgggcagtctttctgctcgcagggtctgccctcctctgtgccgtggtcacgggcgtcatctggaggcagcccgagagcaagaccaagctctcatttaaggttcccttcctgccagtgctccccatcctgagcatcttcgtgaacgtctatctcatgatgcagctggaccagggcacctgggtccggtttgctgtgtggatgctgataggcttcatcatctactttggctatggcctgtggcacagcgaggaggcgtccctggatgccgaccaagcaaggactcctgacggcaacttggaccagt

SEQ ID NO: 181 - DNA, 463 bp, human
gagagattatttctgtggtctaaaggttaaaaagccaacaacctgttaccaattatttcagctttttttgttttaataagtgtgacaacttaaaacttgtttctatttaaagtgaaatgtatctttcaactgtttagttacccagctgtttaatattccagtcttcccaaagtgaaaagatttgtatacaaatgttttctatgatttaataaaaatatatggcacaaaaaaccacttcgccgggtcgcgccccgacggccgggcccgggagacgcgccggcagccccggcaccttgccaaagtttcaaacccgggaaaataaacgtaagctaaggatcccccccatgtatccaacctcatgctctatgggacccaggccatccccgtgaggttctccagatcctccatgccttggacgaaaggtgttggatcactggtgcatcatgacaccaaatctatagtt

SEQ ID NO: 182 - DNA, 482 bp, human
atttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactg

SEQ ID NO: 183 - DNA, 391 bp, human
  misc_feature (295)..(295): n is a, c, g, or t
gataaccacttgaaccaggcaaatacactgaagataaaggttatttctttttttagctttgaatttgtcatgaccattttagtcttgcagatagcagggcagccccttggggcaaggatttactctgggaggtaccgttaagagccttctttccccctttgaaagatccttttacaatgttaaagtatactagttgcaagaacaagcaggatttgcaggttgctttaccagcatgagtctcatttttctggcttaaaatctgggactgtgaaattattccataggaaagtgaatnttattttgcagaattagcctcttacataaaagtatttgttgaagtgtctttaaaattgctatcatgagcaaaactggttgctgtaatgcttgtttt

SEQ ID NO: 184 - DNA, 477 bp, human
ctccttcatgggcaagcgctacatgacccaccacatcccacccagcgaggccgccacactgccggtgggctgcaagcctggcctggaccccctccccagcctcagcccctagcctggcctctttgcatggggctgggggagatggggcgccgggagtgagtgcatggtgctttgtcccagctcctgcacccacaggccccctcagggctccttgcctttcccccccaccagcacaccccgtaccctgcctggaatcccagcaccagcccccctgcctctcctctgcctttctggtttctctccctctccaagcatctgtaagttgcactcaggagggtttaggggagggccatgggcaggctggtctcgtgatagtgagtgagtgctcatgggatctggttgtttagaagcatgcagcacctcctgcttcactctctctgtctctcctgctccaccatggccagaaacgtgcctgct

SEQ ID NO: 185 - DNA, 546 bp, human
ggcaatctgtcacactctcagagtctgggacttgacttgctaccaacaactgctgtgcaattctgctgagcaggaatatcatgagctgttcaataatgacggacgcattggttgagatgaagtttccagtaaggaagtgacagtgcaatgtggatatttatggctgtaaaataggaagagctttagttcccaggctgaacctgccactgctggagccatttcaacaaggcatcctcacaacaaagaagagatgtgatttggtaccatttcacaccagcaggtgtctggacgaaaacatcaatgtgaataagggccaagtgcagtcctgtcttgattaaattacttaataatattattaaataataataggtctgggcagtattgtttttaacctgactcatccagctgtccttcaaatagctccgtctccctctacccagaactgatttttaaaaagaagtaatttttctccctgggctgggaaaaccctaatgaactgaaacacacttttactttaaaatttttctgtctggcgtttttgtaatc

SEQ ID NO: 186 - DNA, 409 bp, human
ttaacttcattttgtatcttgcttaagtagcaggctcactaaaattagagaaagtccaataactctccccctttcccttgagaaatctttaagtttcgattctggagcaaaaactttcagcattaaatatttcagaggctccattcacagctttcagataaactggagtgttcagatggactgttttaataaaaatctttgagcaagtgagttatggcaagagaaactcagcctctttctgtataaacttaacagggaagggctggggtgtgaaaaagaagattgtatgaaaaccattggtaatttttattttttatttttgggactgcactatcctgttcacgaagacatgtgaacttggttcagtccaaatggggatttgtataaaccagtgctctccattagaaat

SEQ ID NO: 187 - DNA, 339 bp, human
ggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctcca

SEQ ID NO: 188 - DNA, 287 bp, human
tatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaa

SEQ ID NO: 189 - DNA, 460 bp, human
agctcttgcaacgtttactggacatgggagagactgacctcatgctggcggctctgcgtacgctggttggcatttgctctgagcatcagtcacgggtaggtggagtggagaggctggttacagcttcagtccctttgtctgtctgtccatccattcctccatccacccatctgtccatccgcccatctgcccgtccatccatccatccactcttccaccctctaccccttaccttattctaaaaagaacttaggtaggttatggtgcctcacacctgtaatcccagcactttggggggctgaggcaggtggatcacttgaggtcaggagttcaagaccagcctggccaacaaggcgaaaccctgtctctactaaaaataaaaaaattagccgggcatggtagtgcacgcctgtaatcccagctactcaggaggctgaggcacgagacttgcttgaaccca

SEQ ID NO: 190 - DNA, 444 bp, human
gaaagccatttgtttggcatgtgagttaaaggcagttccaatgcctgatggttccccagatctatgaaatgggtggaccattaaccttacatgtaaagattatgttagtaattaagaaaccctaacaaaggtgttaccaaggaacctttgggagtgccttttttgtttttcaagatggacccaaaaaaagtggaggaagatattgttcttttgtgccctcctacctgtgagagatatttgtagtcctatgtgaatgagcttatccctccacaaccaggtgcatatgaaagtgtacatattatgactgccaagtattggaaatgaaaagacctggagtctatgctaggaagctgagatattttggtattgcattggtttttatggtaactaggttttgcatgcaattaaaaatccttatttcttgttctagggcttcccttagtt

SEQ ID NO: 191 - DNA, 376 bp, human
ggagcctcaggcactgaaggtcctgagaactgcagagtttgctccttttgttgttttcattgctgcaccaactattactccaggtttaaatgaggatgaatctcttcagcgtctgcagaaggagtctgacatcttacagagaacatatgcacactacttcgatctcacaattatcaacaatgaaattgatgagacaatcagacatctggaggaagctgttgagctcgtgtgcacagccccacagtgggtccctgtctcctgggtctattaggcctctccccagatatctgagcataactgggagcacctcatttgtggaaaagcctctttgttatcggccttgtgtcagcaggtcatggtccctagagactaccta

SEQ ID NO: 192 - DNA, 537 bp, human
gagaacacggtggcagagacggagtgccgctatgccctgcagctgcagcagatccagggactcatcagcagcatcgaggcccagctgagcgagctccgcagtgagatggagtgccagaaccaagagtacaagatgctgctggacatcaagacacgtctggagcaggagatcgccacctaccgcagcctgctcgagggccaggacgccaagaagcgtcagcccccgtagcacctctgttaccacgacttctagtgcctctgttaccaccacctctaatgcctctggtcgccgcacttctgatgtccgtaggccttaaatctgcctggcgtcccctccctctgtcttcagcacccagaggaggagagagccggcagttccctgcaggagagaggaggggctgctggacccaaggctcagtccctctgctctcaggaccccctgtcctgactctctcctgatggtgggccctctgtgctcttctcttccggtcggatctctctcctctctgacctggatacgctttggtttctcaacttc

SEQ ID NO: 193 - DNA, 467 bp, human
ttaggcactttttgaccttgctgtctggatgagtttcctcaatgggatttttcttccctagacacaaggaagtctgaactcctatttagggccggttggaagcagggagctggaccgcagtgtccaggctggacacctgccattgcctcctctccactgcagacgcctgcccatcaagtattacctgcagcgactcaaccctatgcatggagggtcaatgtgggcacatgtctacacatgtgggtgcccatggatagtacgtgtgtacacatgtgtagagtgtatgtagccaggagtggtggggaccagaagcctctgtggcctttggtgacctcaccactccctcccacccagtccctccctctggtccactgccttttcatatgtgttgtttctggagacagaagtcaaaaggaagagcagtggagccttgcccacagggctgctgcttcatgcgagagggagat

SEQ ID NO: 194 - DNA, 358 bp, human
ggagcagtggactgccacaagccaccatgtaacccctctcacctgccgtgcgttctggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcag

SEQ ID NO: 195 - DNA, 467 bp, human; identical to SEQ ID NO: 193

SEQ ID NO: 196 - DNA, 218 bp, human
atgtcttatcaacagcagcagtgcaagcagccctgccagccacctcctgtgtgccccacgccaaagtgcccagagccatgtccacccccgaagtgccctgagccctgcccaccaccaaagtgtccacagccctgcccacctcagcagtgccagcagaaatatcctcctgtgacaccttccccaccctgccagccaaagtatccaccgaagagcaagta

SEQ ID NO: 197 - DNA, 233 bp, human
gcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtcggctgtgccaagtgtgcccagggctgcatctgcaaagggacgtcagacaagtgcagctgctgtgcctga

SEQ ID NO: 198 - DNA, 578 bp, human
cccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacc

SEQ ID NO: 199 - DNA, 277 bp, human
aagaggggcctgatgagactccactcaggtgcacacatcaccaggtgcatctgcaggcaccgggctggctgcttgcagccaggagaaggtcagcgagaaggagtgtatgagtgtgagtgtgtgtgcatggaagttggggcactgggcgtctgactccctccccacccaagagaggaaggacccctcaccacccccactggcgagacagtttactttgccgacttgccatgtttttgccaaaaccaagattttgaaggaaatgagtggccagcgccag

SEQ ID NO: 200 - DNA, 332 bp, human
tggtgagaagggccggcggattcgggaactgactgctgtagttcagaagaggtttggctttccagagggcagtgtagagctttatgctgaaaaggtggccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgggaaactccgaggacagagggctaaatccatgaagtttgtggatggcctgatgatccacagcggagaccctgttaactactacgttgaca

SEQ ID NO: 201 - DNA, 537 bp, human
tgaacagtaccgcctgcagagggagaaagaattcaaggccaaggaagctgcggcattgggatcccgtggcagttgcagcactgaagtggagaaggagacccaggagaagatgaccatcctctagacatacttccggcagaacagggatgaagtcttggacaacctcttggcttttgtctgtgacattcggccagaaatccatgaaaactaccgcataaatggatagaagagagaagcacctgtgctgtggagtggcattttagatgccctcacgaatatgaagcttagcacagctctagttacattcttatgatatggcattaaattatttccatatattatataataggtccttccactttttggagagtagcaaatctagcttttttgtacagacttagaaattatctaaagatttcatctttttacctcatatttcttaggaatttaatggttatatgttgtctttttttcctatgtcttttggctcaagcaacatgtatatcagtgttgactttttctttcttagatctagtt

SEQ ID NO: 202 - DNA, 527 bp, human
tcagtctaactctgtgtgctacgccaagaacgggcaggttatcggcattggagcaggacagcagtctcgtatacactgcactcgccttgcaggagataaggcaaactattggtggcttagacaccatccacaagtgctttcgatgaagtttaaaacaggagtgaagagagcagaaatctccaatgccatcgatcaatatgtgactggaaccattggcgaggatgaagatttgataaagtggaaggcactgtttgaggaagtccctgagttactcactgaggcagagaagaaggaatgggttgagaaactgactgaagtttctatcagctctgatgccttcttccctttccgagataacgtagacagagctaaaaggagtggtgtggcgtacattgcggctccctccggttctgctgctgacaaagttgtgattgaggcctgcgacgaactgggaatcatcctcgctcatacgaaccttcggctcttccaccactgattttaccacacactgttttttggcttgctta

SEQ ID NO: 203 - DNA, 564 bp, human
tagaccatctccatttttagcacttggcagcctcatgatccttttataaatgtgagattaacaggagagcagcaatacgattttgccaatggaataacagatttgccggcattcactgaaagagggcagatattgggtccttgtaacttcaactgactcttccaaattgtatgaatttatcaatgtattacacaaatccagtttcagaatgataaaaaatgttagaccaaataatgcggctaattaacagtcgtatgatttctagcccatgggtttaaaactgtatcttaaagagtcattttaaaataatataaatattaaaaaatgtaactgctatcttaatgttctgaaataaaacattttaaaatataaatcctgtagtttaaaaggaagaaatggtgggaaggaaaagtagagaaagaaatgccaattacaggccaaagcgttatttgccaagttttcttagaatgaattttaccaatgtatgagttcttgttaacagaatgtgtaacggaaatactgaaagacttttgcttaaagtggcattattgactgctgatgtga

SEQ ID NO: 204 - DNA, 464 bp, human
aatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagcca

SEQ ID NO: 205 - DNA, 536 bp, human
ccccccacttttggagatgcgtcagtcattgcccttgaattactaaattctggatatgaatttgatgaaggctccatcatctttaataaattcaggtctgtcatctcctataagacagaagaaaagcccatcttttcccttaataccgttgcaagtgctgacagcatgagtatctatgacgatattgatgctgacgtgctgcaaaattaccaagaatacaatctggccaacatcatctactactctctgaaggagtccaccactagtgagcagagtgccaggatgacagccatggacaatgccagcaagaatgcttctgagatgattgacaaattgacattgacattcaaccgtacccgccaagctgtcatcacaaaagagttgattgaaattatctctggtgctgcagctctggattaatgaaaatcaagttccatcctcagacaagaggtaaagaaggaaaattcagccagttgattttgtttttagcttactgctgcctttgtccgaagaaaccgttcctccattatttgaat

SEQ ID NO: 206 - DNA, 540 bp, human
  misc_feature (373)..(373): n is a, c, g, or t
tgcctgtatttgagactggagctgcctgtatgaggactggatcaactgctagtcacgttatatccaaatctgcattatcattgggcacattttcacagaattttactgaattattccttaattgtttaatggttgggaatagtttgggaattaccttccatcaactctgctaagaaaggaatggattctggtagcaagacaatataattctcctttagtttttcagccagtgctaacacagtaatcaaagcagcaaatcgaacctgaaagggataaaagagcaaagaaataaaaagtagtgttactgtatttattatcttaagagctgtactgacttgagacaagctctaactttttaaacattagttcacangcgtttattcacttcattatgttcattaagctttcatcttagaataccagtttcaccatttgggagctgtttgtaatatgtgcaaccttataaatagtgttttccaaactgtgtcccaggactgcaaatctttaatgtgaaatgtctttttataatctcttccttta

SEQ ID NO: 207 - DNA, 445 bp, human
gagcaactttgagtgaaatctgcaacatggataccatgtatgtaagatactgctgtacagaagagttaaggcttacagtgcaaatgaggcgtcagctttgggtgctaaaattaacaagtctaatattattaccatcaatcaggaagagataataaatgtttaaacaaacacagcagtctgtataaaaatacgtgtatatttactctttctgtgcacgctctatagcataggcaggagaggcttatgtggcagcacaagccaggtggggattttgtaaagaagtgataaaacatttgtaagtaatccaagtaggagatattaaggcaccaaaagtaacatggcacccaacacccaaaaataaaaatatgaaatatgagtgtgaactctgagtagagtatgaaacaccacagaaagtcttagaaatagctctggagtggctctccca

SEQ ID NO: 208 - DNA, 337 bp, human
gaagaggtctttgcatggaataatgaagtaaaacagggactttcaagtagcatctttaccaaagatctgggcagaatctttcgctggcttggacctaaaggatcagactgtggcattgtaaatgtcaacattccaacaagtggggctgagattggaggtgcctttggaggagaaaagcacactggtggtggcagggagtctggcagtgatgcctggaaacagtacatgagaaggtctacttgtactatcaactacagtaaagaccttcctctggcccaaggaatcaagtttcagtaaaggtgttttagatgaacatcccttaatttgaggtgttcca

SEQ ID NO: 209 - DNA, 355 bp, human
gagcttccccaactcataaatgccaattttccagtggatccccaaaggatgtctatttttggccactccatgggaggtcatggagctctgatctgtgctttgaaaaatcctggaaaatacaaatctgtgtcagcatttgctccaatttgcaaccctgtactctgtccctggggcaaaaaagcctttagtggatatttgggaacagatcaaagtaaatggaaggcttatgatgctacccaccttgtgaaatcctatccaggatctcagctggacatactaattgatcaagggaaagatgaccagtttcttttagatggacagttactccctgataacttcatagctgcctgtacag

SEQ ID NO: 210 - DNA, 528 bp, human
  misc_feature (472)..(472): n is a, c, g, or t
aaggaattgtagcaggccccatgaatgaagagaacttttttgaatgggaggcattgatcatgggcccagaagacacctgctttgagtttggtgtttttcctgccatcctgagtttcccacttgattacccgttaagtcccccaaagatgagatttacctgtgagatgtttcatcccaacatctaccctgatgggagagtctgcatttccatcctccacgcgccaggcgatgacccccatgggctacgagagcagcgccggagcggtggagtcctgtgcagagtgtggagaagatcctgctgtcggtggtgagcatgctggcagagcccaatgacgaaagtggagctaacgtggatgcgtccaaaatgtggcgcgatgaccgggagcagttctataagattgccaagcagatcgtccagaagtctctgggactgtgagacctggcctcgcacaggcgcacacacaccgccaancagctcagcattctcccccggcacacttagtgacagtgatgctctgtgctggtacc

SEQ ID NO: 211 - DNA, 364 bp, human
cacttcagaccagccaggtgtcttcccgggccctgccagaccctgctcacattccctctgctggtctgtgctggtctcagaaggccaccgcgcccgcattccactcagccagggtccagctgcagcccccgccacccttccttcccttccctgtcctgggtcatgttgttgccaccctgtgtgacttttgaagctgtaaaatgagcttccagggcttgggtggcgtcggggcagggccgccgaggctgggaggaagcccttctgccttttgctggtgtttctggaatttgctttccctcacctctcacttccttctagaaggagcttcctgactggaaccagagaatgcatgtctgtccacttg

SEQ ID NO: 212 - DNA, 488 bp, human
gagtctcagaatgctcaggaccaaggtgcagagatggacaagagcagccaggagacccagcgatctgagcataaaactcattaaacctgcccctatcactagtgcatgctgtggccagacagatgacaccttttgttatgttgaaattaacttgctaggcaaccctaaattgggaagcaagtagctagtataaaggccctcaattgtagttgtttccagctgaattaagagctttaaagtttctggcattagcagatgatttctgttcacctggtaagaaaagaatgataggcttgtcagagcctatagccagaactcagaaaaaattcaaatgcacttatgttctcattctatggccattgtgttgcctctgttactgtttgtattgaataaaaacatcttcatgtgggctggggtagaaactggtgtctgctctggtgtgatctgaaaaggcgtcttcactgctttatctcatgatgcttgcttgt

SEQ ID NO: 213 - DNA, 318 bp, human
tgctcaccatcagatcaacctctgattttacatcatgatgtaatcaccactggagcttcactttgttactaaattattaatttcttgcctccagtgttctatctctgaggctgagcattataagaaaatgacctctgctccttttcattgcagaaaattgccaggggcttatttcagaacaacttccacttactttccactggctctcaaactctctaacttataagtgttgtgaacccccacccaggcagtatccatgaaagcacaagtgactagtcctatgatgtacaaagcctgtatctctgtgatgatttctgt

SEQ ID NO: 214 - DNA, 385 bp, human
gccgccattgctgatgctgagcagcgtggggagatggccctcaaggatgctaagaacaagctggaagggctggaggatgccctgcagaaggccaagcaggacctggcccggctgctgaaggagtaccaggagctgatgaacgtcaagctggccctggacgtggagatcgccacctaccgcaagctgctggagggcgaggagtgcaggctgaatggcgaaggcgttggacaagtcaacatctctgtagtgcagtccaccgtctccagtggctatggcggtgccagcggtgtcggcagtggcttaggcctgggtggaggaagcagctactcctatggcagtggtcttggcgttggaggcggctttagttccagcagcggcagagcca

SEQ ID NO: 215 - DNA, 177 bp, human
gccgggcgggggaatatacaaagtgaagccacattgccaaacttgcagcagcgattgcagcagttgctgccgctgcgccgcgcctgaagccgcgccgcgcgggccgagggctcctgcagctgctcgcgcgcagtcggaggcggagaaggacgaagactgagactgacacttctgctc

SEQ ID NO: 216 - DNA, 497 bp, human
gtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccg

SEQ ID NO: 217 - DNA, 432 bp, human
ctggctatggctcaccagggcagaagggcacggtcatgggtacactgcgcagcctaggtgctctggccagggccgcggggcccctggtggccgcttcagtgtactggctggccggggcccaggcctgcttcaccacgtggtccgggctctttttgctccccttcttcctcctgcagaagctgagttacccggcacagacgctcaaggctgagtagctgagccactgtgcccaggctgtgggcaccaggcagagtgggagcctaggtcaggcccctgcccactgcctgacccccaccccccgccagtccagggagaccctgtgggtgggggccggcccctaagcaggaagctcaggcagctcctccagacttacttactccttcagtgactccgagctgcagcactccaaggctgtcagggcttctgtttgtt

SEQ ID NO: 218 - DNA, 399 bp, human
taaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactgactgcctggtgcccagcacatgtgctgccctacagcaccctgtggtcttcctcgataaaggga

SEQ ID NO: 219 - DNA, 560 bp, human
agacaacctgacactgtggacggccgacaacgccggggaagaggggggcgaggctccccaggagccccagagctgagtgttgcccgccaccgccccgccctgccccctccagtcccccaccctgccgagaggactagtatggggtgggaggccccacccttctcccctaggcgctgttcttgctccaaagggctccgtggagagggactggcagagctgaggccacctggggctggggatcccactcttcttgcagctgttgagcgcacctaaccactggtcatgcccccacccctgctctccgcacccgcttcctcccgaccccaggaccaggctacttctcccctcctcttgcctccctcctgcccctgctgcctctgatcgtaggaattgaggagtgtcccgccttgtggctgagaactggacagtggcaggggctggagatgggtgtgtgtgtgtgtgtgtgtgtgtgtgtgtgcgcgcgcgccagtgcaagaccgagattgagggaaagcatgtctgctgggtgtgaccatgtttcctctcaataaagttcccct

SEQ ID NO: 220 - DNA, 391 bp, human
atgtaattgcccttgtgtgtagttaaaatgagtcatcatctggtcctttgtgaaatggaattcatggtattttctgtaacgttttcctgaagctgtttctggagagccacacatttaaatacagacagctttcctgatcatttgatttattgtgcacctgatttttggtctaaaaggaattattgccacaatatattttatttattctttagattttagccttgtaagttaaagtgctttacatgatgatgtgaaaagctgtttgtccctttactgggtttggggggttgttaaaagatagggaatgaagaatgcaaaatggtttatcgttcaaactgtccactctgatccaaccctgtactgatagtacttcccagtatgatattgtgat

SEQ ID NO: 221 - DNA, 541 bp, human
gcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagccca

SEQ ID NO: 222 - DNA, 488 bp, human
tctggcttcgacccaacaaagttcattaacctgggatgaatggggttggcctgttggtgatttggatgctgttctgtgatctaaaacaactcttattgaattgtatttactccctaaacaacacttgacaggctgttgcacagggcttctatagatcagtgtgttaggaatgggaggccccttcctgcctgccttcccatattggtcccttgacattgacaaaagcacagtgactgtcagcagattcctttacttttgtttgtgggaggtaggaattgttttaatgcattttaaacagtgtttctgaaattggatggctggctaatagacactgaatcacccggagtgcttatcttaaaattgcagatttagggagcctgccaatttaacagtctcatcagtgatctttcaacagtaatgtttgagaattactgggttaaattgtggaaagggtccagattttaaaggtgctttaaggttgccctctg

SEQ ID NO: 223 - DNA, 262 bp, human
ggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcatta

SEQ ID NO: 224 - DNA, 418 bp, human
cagctccaagacctgttggttccaagccctggagccaactgctgctttttgaggtggcactttttcatttgcctattcccacacctccacagttcagtggcagggctcaggatttcgtgggtctgttttcctttctcaccgcagtcgtcgcacagtctctctctctctctcccctcaaagtctgcaactttaagcagctcttgctaatcagtgtctcacactggcgtagaagtttttgtactgtaaagagacctacctcaggttgctggttgctgtgtggtttggtgtgttcccgcaaaccccctttgtgctgtggggctggtagctcaggtgggcgtggtcactgctgtcatcagttgaatggtcagcgttgcatgtcgtgaccaactagacattctgtcgccttagcatgtttgct

SEQ ID NO: 225 - DNA, 579 bp, human
agtgatgagttgtgtgcttgtccctggcagtggcaggctgtgtcctatggacatcttggcaggacatggaatttggcctcatgacaggcccaactagggataggaaggaaaatgaagagagccagtatttccccttctccagaagcaggtactcagctttctgggaaaagcgtgcctccagccgtggggacaggccatcctactgactacctcttgcttggcatgaaataaactgctatcctccccttggaatctaccgccactctacatcctactgctttggcctccctctcctctcaccagatggcatgtggtgtggcacctgtggctggacacaggaggcctcaggatcacaaatgttacactagacatatgtcctaatgtgctgcccagaaacctcaactgttccccagctactgaggggcactgtcagcgagatgttgggtctggaggtgatgagatcgggccacacttgagctgagtcaccagaccctattgcttcaacagtgcttggcccccgccagcttgtcccagccactctagctgctggatgtgatcctgggacatgtactccaagcc

SEQ ID NO: 226 - DNA, 423 bp, human
ttcactatgtatcctgttactgggcttaaacagctttcagagaagagatgtcatttctattaaatgctctttcagtagcgaactgagttcacacttgactaaggatattttccggactgtctgtcatcagcatccttagtgggtttccccatatttaaattggtagaggccagggatggtggctcacacctgtaatctcagtactttgggaggccaaggtaggtggattgcttgagctcagaagaccagcctgggcaacctggtgaaaccctgtctctactaaaaattcaagttagctagctgggcatggtgatgcacttctgtagtcccagctacttggagagggggtggtgctggggcagcaggatcgcttgaacccaggaggttgaggttgcagtgagccaagatggtaccagcctaggt

SEQ ID NO: 227 - DNA, 505 bp, human
ggaagcacaatttccaccttattttttgaactttggcagtttcaatgtctgtctctgttgcttcggggcataagctgatcaccgtctagttgggaaagtaaccctacagggtttgtagggacatgatcagcatcctgatttgaaccctgaaatgttgtgtagacaccctcttgggtccaatgaggtagttggttgaagtagcaagatgttggcttttctggattttttttgccatgggttcttcactgaccttggactttggcatgattcttagtcatacttgaacttgtctcattccacctcttctcagagcaactcttcctttgggaaaagagttcttcagatcatagaccaaaaaagtcataccttcgaggtggtagcagtagattccaggaggagaagggtacttgctaggtatcctgggtcagtggcggtgcaaactggtttcctcagctgcctgtccttctgtgtgcttatgtctcttgtgacaattgttttcctccct

SEQ ID NO: 228 - DNA, 510 bp, human
tctccattcgatatcagctcgttcgactcttctatgatgtctgagtcccttgatcattgtcctttacctcacagtctctaggattcctgactcaggctgacctctctctctggtcccagactgcctccttgcccaggcctctctcactcttcatactcctccagattttgttctcagcattttcctttctctgtgatcattggcatcctgggcgtttcttgccctctgctgactactgattggattttacctatggctttctgcaacttgctactctctccctctccatcccatctttgcagcctcatagggtgggatacagcagctttttttgcagttatccacactcacatttcagagtcctgactctcaaggaaccactggtttttgggatagaacttgggccagggctaggaacacaggctccacggtgacatgtcatttgattgtaaattaagtgttctgattagtaagaactaagcagggggccacatgctctcaatggagaca

SEQ ID NO: 229 - DNA, 350 bp, human
ccgagagcgccttaactcgagtcggatccgtgttggggcgggcgttgggtttggggggacggtgcccccagcccaggatcgggcactcagtggagccgcacacggcccggcgcgcctggtagagcctcgctggccccgcgccccggagccctatattaaggccacggagcgacagcgggcagtgcgggcctggcgggaggtgggggaggtccatctcagaacaccccagccttgagcttagctgcaggcccaggccctctgctctgctcccgggctaggaggtggccctctgtctgggcgaacagccccctcctcaccgcccgccgtgcaagagtcgagccggcagagca

SEQ ID NO: 230 - DNA, 440 bp, human
atttcagtgcagttcattcttaatggaaaatctgaaacctaaattgcagatttaaaaggtactgtacaaccattatatctgtaaataacttagcacctttttgtcacttagaataatatgtactactacttgagtgagcgcttttggaagttatatcaagttctagtgtttgcttcttagtaactgaactgaatttacagttctgtcctagacattttgcactaaagtagccgaatccactctcatgtcttttcgttaatgtgctctgtaccactggtgagtgctccatagtttccttacctgctgctacagaatgttattttacatccctatggctattgccaaggctacaaaaaaggaaagctatatttgtatgcaacactaaccttttgactgctaatgtatgtttctgcttgctgtgccttgttatggctgctttt

SEQ ID NO: 231 - DNA, 496 bp, human
tctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggcgacttcagtaagctacagctaagcccatcggccggaaaagaa

SEQ ID NO: 232 - DNA, 363 bp, human
gtcgatttacacttacctcggttcaaagtggaagagagctatgacctcaaggacacgttgagaaccatgggaatggtggatatcttcaatggggatgcagacctctcaggcatgaccgggagccgcggtctcgtgctatctggagtcctacacaaggcctttgtggaggttacagaggagggagcagaagctgcagctgccaccgctgtagtaggattcggatcatcacctacttcaactaatgaagagttccattgtaatcaccctttcctattcttcataaggcaaaataagaccaacagcatcctcttctatggcagattctcatccccgtagatgcaattagtctgtcactccatttgg

SEQ ID NO: 233 - DNA, 336 bp, human
gcaacaacagattctcttggctcatatttcttttctttctcatcttgatgatgatcgtcgtcatcaagaatttaatgattaaaatagcatgcctttctctctttctcttaataagcccacatataaatgtactttttcttccagaaaaattctccttgaggaaaaatgtccaaaataagatgaatcacttaataccgtatcttctaaatttgaaatataattctgtttgtgacctgttttaaatgaaccaaaccaaatcatactttttctttgaatttagcaacctagaaacacacatttctttgaatttaggtgatacctaaatccttcttatgt

SEQ ID NO: 234 - DNA, 388 bp, human
aaagaaggaacctactcactgcccaagagcgaattcgttgtgcctgacctggagctgcccagttggctcaccaccgggaactaccgcatagagagcgtcctgagcagcagtgggaagcgtctgggctgcatcaagatcgctgcctctctaaagggcatataacatggcatctgccacagcagaatggagcggtgtgaggaaggtcccttttcctctgttttgtgtttgccaaggccaaactcccactctctgcccccctttaatcccctttctacagtgagtccactaccctcactgaaaatcattttgtaccacttacattttaggctggggcaagcagccctgacctaagggagaatgagttggacagttcttgatagcccagggc

SEQ ID NO: 235 - DNA, 470 bp, human
atgacttagattgtgaccttttagattcggtgttgagctctgtgttgtattacttcctaaaagataatgcttaaacattaagcattagtgtgctcttcatgttaatatggcagagttttgtaaactaaattaaaacttactgatatattggactttgagccaagggaaagaatgagtactatctttccagatatcttaagggtaaaagcttattctaagacagtctgtccattgagaatattagatttctgacttgcaaatatgtttgtactccagaagaattagaggaaaagcagatactagaattctaatttaattacatatacagccgtctttgtttatagtgtagaattctttatattttgtacaaaaactaattcttttggtaaaatgaaccatttacagttcggttttggactctgagtcaaaggattttcctttaaatgcttgtctcaattttagtctggtct

SEQ ID NO: 236 - DNA, 531 bp, human
tcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtg

SEQ ID NO: 237 - DNA, 337 bp, human
agagcagccgcagtgaggtgacggagctccggagggtgctccagggcctggagattgagctgcagtcccagctcagcatgaaagcatccctggagaacagcctggaggagaccaaaggccgctactgcatgcagctgtcccagatccagggactgattggcagtgtggaggagcagctggcccagctacgctgtgagatggagcagcagagccaggagtaccagatcttgctggatgtgaagacgcggctggagcaggagattgccacctaccgccgcctgctggagggcgaggatgcccacctttcctcccagcaagcatctggccaatcctattc

SEQ ID NO: 238 - DNA, 528 bp, human
aacactagggccttggaaattcctgtactgtgtctcatggatttggcactagccaaagcgaggcacccttactggcttacctcctcatggcagcctactctccttgaggatgagtagccagggtaaggggtaaaggatagtaagcatagaaaccattagaaagtgggcttaatggagttcttgtggcctcagctcaatgcagttagctgaagaattgaaagtttttgtttggagacgtttataacagaaatggaagcagagttttcattaatccttttacctttttttttttcttggtaatcccctaaaataacagtatgtgggatattgaatgttaaagggatattttttctattatttttataattgtacaaaattaagcaaatgttaaaagttttatatgctttattaatgttttcaaaaggtattatacatgtgatacattttttaagcttcagttgcttgtcttctggtactttctgttatgggcttttggggagccagaagccaatctacaatctctttttg

SEQ ID NO: 239 - DNA, 556 bp, human
ggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 240 - DNA, 541 bp, human
gaccaatgacccttgccttggaaataaatgcgtacatggcacctgcttgcccatcaatgcgttctcctacagctgtaagtgcttggagggccatggaggtgtcctctgtgatgaagaggaggatctgtttaacccatgccaggcgatcaagtgcaagcatgggaagtgcaggctttcaggtctggggcagccctactgtgaatgcagcagtggatacacgggggacagctgtgatcgagaaatctcttgtcgaggggaaaggataagagattattaccaaaagcagcagggctatgctgcttgccaaacaaccaagaaggtgtcccgattagagtgcagaggtgggtgtgcaggagggcagtgctgtggaccgctgaggagcaagcggcggaaatactctttcgaatgcactgacggctcctcctttgtggacgaggttgagaaagtggtgaagtgcggctgtacgaggtgtgtgtcctaaacacactcccggcagctctgtctttggaaaaggttgtatacttcttgaccatgtgggact

SEQ ID NO: 241 - DNA, 441 bp, human
ttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaacccccttttttttgtcttaatgttgcacataagtttatacagagtggatgaccacactagcacagaagagaacaacatgtattaaagcaggtgattcctcccttggcgggagagctctctcagtgtgaacatgccttctgtgggcggaaatcaggaagccaccagctgttaatggagagtgccttgcttttatttcagacagcagagttttccaaagtttctctgctcctctaacagcattgctcttt

SEQ ID NO: 242 - DNA, 532 bp, human
ggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaata

SEQ ID NO: 243 - DNA, 460 bp, human
atctgaatgacacggaacgctcccactgcaggcaaaccgtgacgccctccccactcgggagaagcagagctgaccttaggaccgagcaccagggcaggttgcgctgactctgcggccctccaggacggacaccgggtgaccccttaggcaccaggcaagatccctaagaggcacccaatgcccaggccagggggctgcagccctcagcccccgccaggattccgcaggctcctggactggaagctccctccgcggtcggattctggagtgtgggaggcatcttggcctgcagtaagcggtgctgacggggactctggccacagaggtcaggcctcctgaaaacagcactgccttccgcgctgccccagcttgccccattccttgtccgccaacccaccgtgattcatcttctgaagctgggagtgaaactgggtcagctgtaacctgttcctattcatct

SEQ ID NO: 244 - DNA, 409 bp, human
agtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgcccc

SEQ ID NO: 245 - DNA, 497 bp, human
tggcctcgtcaatgctgtgttatgaacaatcttaaagaacctctcaacctggaggcttgtaaactaggcgtgcctggtttttatcacaatcagggctgctatgaactgatctctggtccaatgaaccgacacgcctggggggttgcctggtttggatttgccattctctgctggactttttgggttctcctgggtaccatgttctactggagcagaattgaatattaagcataaagtgttgccaccatacctccttccccgagtgactctggatttggtgctggaaccagctctctcctaatattccacgtttgtgccccacactaacgtgtgtgtcttacattgccaagtcagatggtacggacttcctttaggatctcaggcttctgcagttctcatgactcctacttttcatcctagtctagcattctgcaacatttatatagactgttgaaaggagaatttgaaaaatgcataataactacttccatccctgc

SEQ ID NO: 246 - DNA, 565 bp, human
gtttagtagcctcaattctccattaattaaaagtgtgggctgggcgtgggggctcatgcctgtaatcccagcactttgggaggccgaggtgggcagatcacctgaggtcaggagttcaagaccagcctggccaacatggtgaaaccccgtctctacaaaaatacaaaaattagccaggcgtgatggcaggtgcctgtaatcctagctacttggcaggctaacgcaggagaatcacttgaccgggagacagaggttgcagtgagctgagatcgtacctattgcactccatcctggatgaaagagccagactctgtctcaaaacaaacaaaaaagcgtggggacttctggggacagacaaggtgcctgttatatatttactcagtctttgccctgaatggtctcagcttgagaccatttcaaactggagagaagcaagccagccaatagaatggggtgatttacagggatttctgtttactgtcaaaatatttctcatctgcactatgtttccatttgtggtcctgaaggaaattcttataactcaacatttgtctggtcttataag

SEQ ID NO: 247 - DNA, 538 bp, human
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccggggatgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagggactcatgccagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctggggtgt

SEQ ID NO: 248 - DNA, 461 bp, human
accccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccataaataatcaccatttttgaaaccaaaccagagcctgagtgttgcctaatttgttttcccttcttacaatgcattctgaggtaacctcattatcagtccaaagggcatgggttttattatatatatatatattttttttttaaaaaaaaacgtattgcatttaatttattgaggctttaaaacttatcctccatgaatatcagttatttttaaactgtaaagctttgtgcagattctttaccccctgggagccccaattcgatcccctgtcacgtgtgggcaatgttccccc

SEQ ID NO: 249 - DNA, 522 bp, human
tctatcccgtcaaattcaccttaaataacacccagctagatacaggcactaggtttgtgtaagatatgttgatacacacgaacaaagtttattttgactataatgtgtggactgactttcaacatttgcattttatctcacaaaggtgtatctattcaagtaacctttttttttttgtttgtttgtttcttttttgtttttttttttcttttggttgtttgtttcaattcatgtagctatttaaactgggataccttggactaagccagtctgtatcccaattcgctagcaagcctaagtttgtggggttttgtttttgtttttgttttaccttctaatttacaagaaagaggaaaagctcttctaactgaactttggtatgcggttgagctttgtaactatttgttctccatgaaaacaaaattatttatatttgacatatttttttctagtgtattaagttattttaaacaaaagatgttatctcatgacgtgttgtcagtacaaaatgtgtcgcctcca

SEQ ID NO: 250 - DNA, 492 bp, human
gcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaagaggcgtgctgtgggcgctgtgggccaggtctcgggggctccgggaggtgctgcctgccaggggaagctggaaccagtagcaaggagcccgggatcagccctgaactcactgtgtattctcttggagccttgggtgggcagggacggcccaggtacctgctctcttgggaagagagagggacagggacaagggcaagaggactgaggccagagcaaggccaatgtcagagacccccgggatggggcctcacacttgccacccccagaaccagctcacctggccagagtgggttcctgctggccagggtgcagccttgatgacacctgc

SEQ ID NO: 251 - DNA, 445 bp, human
gagctcgattcacggaggcattgaaattttcagcagagaccttccaaggacatattgcaggattctgtaatagtgaacatatggaaagtattagaaatatttattgtctgtaaatactgtaaatgcattggaataaaactgtctcccccattgctctatgaaactgcacattggtcattgtgaatatttttttttttgccaaggctaatccaattattattatcacatttaccataatttattttgtccattgatgtatttattttgtaaatgtatcttggtgctgctgaatttctatattttttgtaacataatgcactttagatatacatatcaagtatgttgataaatgacacaatgaagtgtctctattttgtggttgattttaatgaatgcctaaatataattatccaaattgattttcctttgtgcatgtaaaaataac

SEQ ID NO: 252 - DNA, 527 bp, human
gatgtttcgttgaatctatttagagcttcaccatggcaatatgtatttcccttaaaacactgcaaacaaatatactaggagtgtgcccttttaatctttactagttattgtgagactgctgtgtaagctaataaacacatttgtaaaaacattgtttgcaggaagaaaacttcgagttacaggtcaggaaaagcctgctgaatttatgttgtaaacgttacttaacacagtataaagatgaaaagacaacaaaagtatcttcatacttcctcatcccctcattgcaacaaaaccttaaactgggagaaccttagtcccctctctttcctcttcctcctccacttcccacttattgccactttgtaatattcagagagcacttggattatggatctgaatagagaaatgcttacagataatcattagcccacataccagtaacttatacttaaagatgggatggagttataaagtgcttttataatccaatataattgctaaaggcaagggttgactctttgttttat

SEQ ID NO: 253 - DNA, 543 bp, human
gcaggaggtctgtgttgtcatggtgaactggagtttctcttgtgagagttccctcatctgaaatcatgtatctgtctcacaaatacaagcataagtagaagatttgttgaagacatagaacccttataaagaattattaacctttataaacatttaaagtcttgtgagcacctgggaattagtataataacaatgttaatatttttgatttacattttgtaaggctataattgtatcttttaagaaaacatacacttggatttctatgttgaaatggagatttttaagagttttaaccagctgctgcagatatatatctcaaaacagatatagcgtataaagatatagtaaatgcatctcccagagtaatattcacttaacacattgaaactattattttttagatttgaatataaatgtattttttaaacacttgttatgagttaacttggattacattttgaaatcagttcattccatgatgcatattactggattagattaagaaagacagaaaagattaagggacgggcacatttttca

SEQ ID NO: 254 - DNA, 511 bp, human
atgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaagagggtttcaatatacatctacatactatatatatattgggcaacttgtatttgtgtgtatatatatatatatatgtttatgtatatatgtgatcctgaaaaaataaacatcgctattctgttttttatatgttcaaaccaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatt

SEQ ID NO: 255 - DNA, 278 bp, human
gggtcagatggtcccatgggtggattaggaggaatggagtcacatcacatgaatggctctttaggctcaggagatatggacagtatttccaagaattctcccaataatatgagcctgagtaatcaaccgggcactccaagggatgatggcgaaatggggggaaatttcttaaatccttttcagagtgagagttactcccctagcatgacaatgagcgtgtgatccattaccaagtctcctcatgaaaaccacagtgagtcagcccttcacagaactac

SEQ ID NO: 256 - DNA, 306 bp, human
cggctgtacgactccataatgggcatggggactcaagataaggtcctgatcagaatcatggtctcccacaatgaagtggacatgttgaaaattaggtctgaattcaagagaaagtatagcaagtccctgtactactatatccagcaagacactaagggtgctgtacctgtgtggtggagatggctgaagtccgacacagcacgagcgtccagaaatggtgctccccatgcttccagctaacaggtctagaaaacccgcttgtgactagcagtccctgtggctgttcctgtgaggatgacgttagca

SEQ ID NO: 257 - DNA, 512 bp, human
atttcaacaatctctatggcaccaaaggtaactacacctcccgggtctgggagtactcctccagcattcagaactctgacaatgacctgcccgtcgtccaaggcagctcctccttctccctgaaaggctatccctccctcatgcggagccaaagccccaaggcccagccccagacttggaaatctggcaagcagactatgctggtggtttaagcaaggtctgagcatccctcaagtcctgctgtggaccacacaaaggcaaggagctgaccaggaggtgtcagttccctgagagacagggccaaaggcagtgtacccttgagtctcactaccggccattctacgtcaacaaaggcaacgggattgggtccaacgaagccccatgagctcctggcggaagggtgctcaaggattggagaggagacaaaaccaggagcagcacagtggggacatctcccgtctcaacagccccaggcctatgggggctctggaaggatgggccagcttgcaggg

SEQ ID NO: 258 - DNA, 528 bp, human
agccctgcgttgtgtgttttcagatgagttactgttaacaggtaggttcgtgtaggccttgctgggcactctgtacaattagttgcttattacgtatgattactcgcagcgatctattgttccatataaccaaaaagcatggtttattcattgaaacacggttgacctgaactcgtgccttaggaattaatgcccccttatggaacctgcctgaattgcacctgcgggtggaggctccggctgtgaagtcactgaacagaacgtcgctgatggagaaagggctcccgcagaaggaacggcctgtaccgtgcgctccggcacaatcgcgtctcttgtgtctcactcacggaaagaaacaacctgaaggccatcccgtcggtctgcacgtaaccgtgaagacgtgtggccgcgtcccacctgcggctgggtaccctgcacccggcactgtaggagtcacgtgcagcctttctcaggggactgtcattgaaaaggaaacgtttgatgtctgtgtcagctgtctttgtagtt

SEQ ID NO: 259 - DNA, 452 bp, human
gagttgcacttattgaccattttttaatttacttgttttggatggcttgtctatactccttcccttaaggggtatcatgtatggtgataggtatctagagcttaatgctacatgtgagtgcgatgatgtacagattctttcagttctttggattctaaatacatgccacatcaaacctttgagtagatccatttccattgcttattatgtaggtaagactgtagatatgtattcttttctcagtgttggtatattttatattactgacatttcttctagtgatgatggttcacgttggggtgatttaatccagttataagaagaagttcatgtccaaacggtcctctttagtttttggttgggaatgaggaaaattcttaaaaggcccatagcagccagttcaaaaacacccgacgtcatgtatttgagcatatcagtaacccccttaaatt

SEQ ID NO: 260 - DNA, 176 bp, human
ggagtgagcctcaccatgtgagctcttcctatccctctcctaactgccagccccctaaaagcactcctgcttaatcttcaaagccttctccctagctcctccccttcctcttgtctgatttcttaggggaaggagaagtaagaggctacctcttacctaacatctgacctggcatc

SEQ ID NO: 261 - DNA, 482 bp, human
agcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcagagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggg

SEQ ID NO: 262 - DNA, 362 bp, human
agttagctttgtaagaaacagtcaaactgttttcccaacgtgacattttatattcccaccaggaatgtttaaaactagtgtcttcaaatcctcaccaacatccaggattgtgtctttatgattatagccatttttgtaggtacaaagtggcatctcatggtggttttaatttgcatttccataatatctaattaggttgagctttttttatgtgcttattggccatttgtttgactttgtttggtgaaatgtatacaaatcatttgctcatttttaatttgggttgtctgtcttgtcttctcattttattgagttaaatgagttcttaataatctctggcttacaagtccttaatttatcaa

SEQ ID NO: 263 - DNA, 473 bp, human
acttggtgtcatgcacctaccagctggccagacggatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcga

SEQ ID NO: 264 - DNA, 496 bp, human
aattacatttcctactgcagtatttgagcagggacagtcattttttaaatgtttttggccgggcgtggtggctcatgcctgtaatctcagtacattgggaggccaaggcaggtggatcacctgaggtcaagagttcgaggccagcctggccaacatggtgaaaccctgtctctactaaaaatacaaaaaattggccgggcgtgatggtgggcgcctgtaatcccagccactccagaggctgaggcaggagaatcgcttgaacctgcgaggcagagattgcagtgagccaagatcaagccattgtactccagcctggacaacaagagcgaaactctgtctaaaaaaaaaaaaaaaaaaaacacacacacacacaacacaatgttttcacgcctgtaaacctagcacattgggaagccaaggtgggaggattgcttgaggccaggagttcaaggctgcagtgagctatgattgcacactgtactctagcctgggagac

SEQ ID NO: 265 - DNA, 195 bp, human
gccggctcctgcaagtgcaaaaagtgcaaatgcacctcctgcaagaagagctgctgctcctgttgccccctgggctgtgccaagtgtgcccagggctgcatccgcaaaggggcttcggaaaagtgcagctgctgtgcctgatgtcgggactgccctgctctcggatgaaaacagaatgacacgtaaagtccggga

SEQ ID NO: 266 - DNA, 545 bp, human
atatgatcatcatgcttccggacgagaccactgacttgagaacggtggagaaagaactcacttacgagaagttcgtagaatggacgaggctggacatgatggatgaagaggaggtggaagtgtccctcccgcggtttaaactagaggaaagctacgacatggagagtgtcctgcgcaacctgggcatgactgatgccttcgagctgggcaaggcagacttctctggaatgtcccagacagacctgtctctgtccaaggtcgtgcacaagtcttttgtggaggtcaatgaggaaggcacggaggctgcagccgccacagctgccatcatgatgatgcggtgtgccagattcgtcccccgcttctgcgccgaccaccccttccttttcttcatccagcacagcaagaccaacgggattctcttctgcggccgcttttcctctccgtgaggacagggcagtcttggtgtgcagcccctctcctctctgtcccctgacactccacagtgtgcctgcaacccaagtggccttatccgtgcagtggtggca

SEQ ID NO: 267 - DNA, 518 bp, human
cggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacc

SEQ ID NO: 268 - DNA, 403 bp, human
  misc_feature (40)..(40): n is a, c, g, or t
ttgtcatgacacatttgccaaatcagtaggatatatttgnttttggcagcctatcacgcagaggctagtggtatatttatgtaagaaaatgactgtaaatctcaagnaaaatctcagcagctaatagcaactcatttatttcattttggtcttaatgctttgtaaacaggtcaaaaaatactgtcatactctaagcttctattttccacactggacatacttctagttgtattctccatactattagactgtgtagtgatgtgacttccaagtagaatttaatctccccattgagtgtgtcatggtacaaatcactattcgtttttggtgttttttagggatgtgcaatgtgcattacataatgacagaaatactgagaaggttctgtgtgcccatttgaaag

SEQ ID NO: 269 - DNA, 476 bp, human
gctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctg

SEQ ID NO: 270 - DNA, 421 bp, human
gagtattacattggccttgggggacagaaaggaggaagttctgacttttcagggctaccttatttctactaaggacccagagcaggcctgtccatgccattccttcgcacagatgaaactgagctgggactggaaaggacagcccttgacctgggttctgggtataatttgcacttttgagactggtagctaaccatcttatgagtgccaatgtgtcatttagtaaaacttaaatagaaacaaggtccttcaaatgttcctttggccaaaagctgaagggagttactgagaaaatagttaacaattactgtcaggtgtcatcactgttcaaaaggtaagcacatttagaattttgttcttgacagttaactgactaatcttacttccacaaaatatgtgaatttgctgcttctgagaggca

SEQ ID NO: 271 - DNA, 384 bp, human
gttcatcaccatggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtgtcgatatttggagtggttca

SEQ ID NO: 272 - DNA, 406 bp, human
ggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagggggccaagactattagccgacgtattactatggttcggggagttatccgagtttgactactg

SEQ ID NO: 273 - DNA, 532 bp, human
caggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatagagggtgttgcacgacatctagcag

SEQ ID NO: 274 - DNA, 546 bp, human
tgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttat

SEQ ID NO: 275 - DNA, 286 bp, human
aaaggagccatgtgccatggcatataagtcagtgtagactggtgccatttgttgtcagactataggtgtggaggtgaaattacaggttcaacagtaattgggacagaaactccaggtaaatggggagtggagaagactgcagtaaattagatggaatgactcttctaaaagttcatctacaaattttccagtgaatatggttgtgtaggatcagtgcatacagaaattctcaggatcttctgtttactatcgctgagatcattatcagaaaatagtctggccgggc

SEQ ID NO: 276 - DNA, 378 bp, human
acatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgca

SEQ ID NO: 277 - DNA, 252 bp, human
aaaacttcacatttatgtggcttgtttatccttagctcacagattgaggtaataatgacactcctagactttgggatcaaataacttagggccaagtcttgggtctgaatttatttaagttcacaacctagggcaagttactctgcctttctaagactcacttacatcttctgtgaaatataattgtaccaacctcatagagtttggtgtcaactaaatgagattatatgtggactaaatatctgtcatata

SEQ ID NO: 278 - DNA, 265 bp, human
tgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtct

SEQ ID NO: 279 - DNA, 498 bp, human
  misc_feature (47)..(47): n is a, c, g, or t
aactgttctgatgaccacacagtgtgatttctttagcagagaaagtnggttttaaaaataaatagtaccacttttctaagactgtacagtttacaaataaggtttttttctttgntgttttcctcttctattaagttttagtgaaaagcctaattacagaaaattgtgcagatactagtgaagatactagtataagtttaaaggaacatgtgactgtaaaatctcacatttacaaagtgcttgatctcttcatatttcacacgcatgttttagaatagattttagggagtgtttaattcattatccttttgacttaaaatttttgttaccaacttcctaggacttagataatatataaataagtacaaatcccaggggaagtgttgtgatgctagactaaaaggtgggaatgtgctgctgttccgtgagccttgttccattgttgaaaatttgatgcctcagtgtttattcagtaccacctcatggagcttcaatgta

SEQ ID NO: 280 - DNA, 402 bp, human
  misc_feature (143)..(143): n is a, c, g, or t
aaagcttgtcaatcactcatgtgttttagagtaattacttttaaaatggtgcatttgtgcttctgaactattttgaagagtcacttctgtttacctcaagtatcaattcatcctccatacatttgaattcaagttgttttttngtcaaatttacagttgtcaattgatcttcaagctgcagggtgcctagaaatgggccgttgtctgtagccctggcatgtgcacacggacatttgccaccactgcaagcaaaagtctggagaagttcaccaacgacaagaacgattagggaaaatatgctgctgtgggttaacaactcagaaagtccctgatccacatttggctgtttactaaagcttgtgattaactttttggcagtgtgtactatgctctattgctata

SEQ ID NO: 281 - DNA, 466 bp, human
taagcattttagggtgggagggactattaagtaattttaagtgggtggggttatttagaatgttagaataatattatgtattagatatcgctataagtggacatgcgtacttacttgtaaccctttaccctataattgctatccttaaagatttcaaataaactcggagggaactgcagggagaccaacttatttagagcgaattggacatggataaaaaccccagtgggagaaagttcaaaggtgattagattaataatttaatagaggatgagtgacctctgataaattactgctagaatgaacttgtcaatgatggatggtaaattttcatggaagttataaaagtgataaataaaaacccttgcttttacccctgtcagtagccctcctcctaccactgaaccccattgcccctacccctccttctaactttattgctgtattctcttcactctatatttct

SEQ ID NO: 282 - DNA, 476 bp, human
actactgtacctttcattggcgcattaccccataaaactttttgagacgaggtgagatctgagtataaagataggtcagaagtattttaaagggcttaatgtgccaaaaagaaaaaaagctagagaccctttttgcaaacatttggtgaccacacatttgagggaagacgtggcgttaggtgaagcagaagcaaaccctgctcttaggggctcacctaggtgagtgcacagcctgtgacgctacagggagaggctgagtaaaccgagatccagcgttctgtatggcaggggtattgcttatcacagaggttctgaagagtaggaagtacataatgaagagggctttaaaaattgccaacaaagtgagtcaccagggctggcagtagtgtgacggggctgtcctgagctgttaggagagtagatgcggggagggctggtgacctccgtgggtttatatgtcggaaactcttctctcc

SEQ ID NO: 283 - DNA, 547 bp, human
  misc_feature (214)..(214): n is a, c, g, or t
gagatcctcgattactggggacccaattccggcccattgacgtggcgcctcacccctgctgaagtgcgccaggtgctggccctgcggatagacttccgcagtgaagatatcaagaggctgcgcctgtagctgcctggatgagcacacctggctcatcacacttgcaggcctgttccctaaggggccccagccaaggagctgagcgaggctgtcnggcttgggggagatctgacagcccagacctttctacggctggcagcagagaaacaaagtctggacccactccatgctctgccctcagacctggccaggtgatgctctgggggcagcatctccccaccgagagaagcgggctcctaatgaggtgggaaagccacggcaggcagcgagcagcccaggccagctttctgcatggatggtcagtctcttgccctcaaacactacagcaaacaagctacccctgccagtcctagacaacttgggtacatctggggacctagcagttaggcttgactttgaggagaggctgtgatgtttatgatccctg

SEQ ID NO: 284 - DNA, 466 bp, human
  misc_feature (111)..(111): n is a, c, g, or t
gccatccacataacctggtgttcacgagaacacactaaaggactcctgagtcactaccacagccacctggaaaccacaaggcatttgatgctaccgttctggtcagggatntgggctgcttcttcagttcctaataccagaccaagcctcctgatgcctttctgcactgcaactgtgtgattgaaaaatgagatgttcatccaagcagtcaagccacagaaacccagcatgtccctgtcacaatctcatgggcaccttgatcatgtcttaaccttcccttaaccttggggctcccaagccagagtcaaggtctgacgccacctcaaggtgacagctcatctccagcacagcacaggcgtgtgcacacagaggtgttccttgcagccccctccctctcaggtgtcctgagatgctgctcctgggagccccctcagaaaactgcctcacctgagacaagtgcctgctg

SEQ ID NO: 285 - DNA, 372 bp, human
  misc_feature (119)..(119): n is a, c, g, or t
tgcagtcccagctcagcatgaaagcatccctggagggcaacctggcggagacagagaaccgctactgcgtgcagctgtcccagatccaggggctgattggcagcgtggaggagcagctnggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctgaggactcagctac

SEQ ID NO: 286 - DNA, 462 bp, human
ttggtgtaagcaacctgtggcctgcacttgtggcctcgaaggaagcacaaaccctccatccacttcccatttcctctgcccttttccacctcccccttccatcccaccagctgccagtggctcccagaaagccttattgagccccttgttgacacttggggctgcggaggcctctccctactggtctggcctttcctgagaggcaggtcttccgtcctcagagcctttctggaacaaggagaatgcctgtgcaggtggacacacaggcctggcctgtcgctctcacttgtcttccagcggggagcttcacgttgccgagtggaagaaccatgacctccacttgcttccaaggtgctagggaagtttcagggtacgctggttcccctctccagctggaggccgagtttctggggactgcagatttttctactctgtgatcgattcaatgcccgatgcttctgt

SEQ ID NO: 287 - DNA, 538 bp, human
gagaactttttaactattcattgactaaaaatgaacattaatgttaaagacttaagactttaacctgctggcagtcccaaatgaaattatgcaactttgatatcatattccttgatttaaattggcttttgtgattgagtgaaactttataaagcatatggtcagttatttaattaaaaaggcaaaacctgaaccaccttctgcacttaaagaagtctaacagtacaaatacactatctatcttagatagatatatttttttttatttttaaatattgtactatttatggtggtggggctttcttactaatacacaaataaatttaatcatttcaaaggcattctatttggtttagaagttgattcccaggagtgccatatttcagctactgtatttcctttttcttgtaatgtaagcagctcagataccatgtgctatcatttttgtatcaagttttttgcacaggatgtgaccactgtcagatcactgttcttttctttctttttgtgattgaaaagcctatactacaatttgaag

SEQ ID NO: 288 - DNA, 390 bp, human
gtctcatgttttattggaccaaagttgtggtttgtatggagtgtagtagtagtgtgtacaggtagaaaacttttaaatacagcatgcaggtgtttcagttagcttgttttcatcaccataactgcaaagatgtggcttagttgtattgcatgcttcctataatttaactctccataattgatgcctgcagtagtgtaaggcatttcatactagtctcctctagtagacctgtgacttactgtgttggacatattatttagacttagtcatacaaagaaacttagctcttttttcatctcacagtaaagcctatttccccaggaaaaaaataaatgcctttgaatgaaaattctgaaattgtaaatgtctattttaatattcacctatgaa

SEQ ID NO: 289 - DNA, 429 bp, human
ggaaggtacgataatcccacaccatatcttggatttcttggaaattgactcaactctccattctaataacatctccattctccaggaactgtacgaaaggtgctggagccacagtaacgaggagtccttcagcccctgctccttggcctggctttacctgcacttgcggcttctctggggtgctatcctgcactcagccctgatctactttctgggaacctttctgctatccatattgatcgcctggactgtgcagtatttccagtctgtctcagcaagcgatccccctccaagaccatcccaggcctccccagacactgccacgtccactgcaagtccagctgtgactccagctgcagatgcctctgaccaagaccagcccacagtaactaataacccggagccacgtgggtgaactgtgcactccag

SEQ ID NO: 290 - DNA, 535 bp, human
  misc_feature (327)..(327): n is a, c, g, or t
tattttggtacctgtgcttgccacagccctgttcctcaaagctgaattgatagatttctctttgacttccaagacctagcagttataaggcaccttgaaataaattgtttgtgcctggaaatgcagggagggcaatagctttgtaaattggtttacatttttctccttgaatttttctagggtcctagtgcttccgaatcatttaatggcattgtcggatatcttttacatttcaattgcaatccatgaaattacatttagaagattcttagtacttaactgtagtcttctccatgaattacacgttagaatagactggcagcaacngaatatgcagcaagtaagcctctagcttatagtttcatccctacccctcatgcctgcgtgagtctgtacagggatatgtgtgtgtgtgtgtgtgtgtgtgtgttagagaggaagaggaagagcagaatgtctgtatactacatgctgctaaggtagtgaataaatcagtaatgcaatattgtgggtccaaactactctttgcactact

SEQ ID NO: 291 - DNA, 507 bp, human
tttccttatctgaggctacctggggattgtgggcagcaggcccctggactcccagaaatgctgagggtccctcttccaggggagttccctggggagcaagagtagaggggctattcccgagggtcctgtggtcagggtcctgctttgtccctggcaggctgtcagtccagctggagggggtcagtaggctccagggagtgggccctcccctccgatcttggagtctctgggtgttccttcagcctcagcctcactggtaccttctgccctttaggggaggctatgagtctgggacaggaggacaaattcagagcccagagggggaaatggagaaacctgggctgctgaggaaagccacgggtgttggcgtgaactgatgatgtctcatcccacgcccactatggccacattacctaacctctccatatctgggctattcaaggtcttggaggccagcacagacccagctagttgttgaagccagttcttttttgtcttggtcatgag

SEQ ID NO: 292 - DNA, 491 bp, human
  misc_feature (26)..(26): n is a, c, g, or t
gtaaattcctcatatcactacagtgncgattattctagaaatcgttgcttgtgtagcaaagaccaaataaatagatttcagacacaaccttgagcacagttgattttggacagctgctgtttattaggaaagggctccaggtggcaaaggtgcacacttcctcagacacaggtgagaagatgcagcaccttccacaggtgaatgggacggattcgaagtgagcaaagggattcacaaattatgtatttatttgttttcatagttaagtagctgaagctcagaggctttcagcaacagagatgaaagtgtggctttttagttttgtgaatggatgatcacaaagaaaaagcatttttaaaaagttggcaaacgctgaaacgcactgtggtatgaagcgcattgcatttccatagcactgaagtaccagtttccattcctgggctgagattgtttttcccgtggttgtattgttctgatttcacgtacaccag

SEQ ID NO: 293 - DNA, 405 bp, human
ctgttttttgtttgtcacctctatttagttgttacctgtttctctcttctttcaccccttgtccttttccacccttttaagagttacgctagcagatcttactccacgtatactttttggtttgtgaaggcatcggttaagggcacaaagacagccatggggacatttatgtaaatacgtctctaattgccacactgcagctgaacagtgtgtagtattttcccagtcagctttgccatactgacgtcaatcatttgagagaaattattcagattttatttttgtatctgtggtaacaaaacattaaccaaaagattttctgtccagaagcctccccgaccccccaagctatttgctcacattaacaaattaaagtgcctgaagcataattcattctttacctgt

SEQ ID NO: 294 - DNA, 379 bp, human
  misc_feature (99)..(99): n is a, c, g, or t
tccccaagcatgactacaactacccctacgtggactcctcagactttggcctaggcgaggaccccaaaggccgcgggggcagcgtgatcaagacgcagncctcccggggcaagtcgcggcggcggaaggaggacggagagcgctcgcggtgcgtgtactgcagggacatgttcaaccacgaggagaaccgccggggccactgccaggacgcncccgactccgtgagaacttgcatccgccgggtgagctgcatgtggtgcgcggacagcatgctctatcactgtatgtcggaccccgagggagactatacagacccttgctcgtgcgatactagcgacgagaagttttgnctccggtggatggctcttattgccttgtc

SEQ ID NO: 295 - DNA, 515 bp, human
ctgctctttgacactgcttttggtaccctgggtggcctatttcaggtggtttttagtgtctgcaagcggattggctacaaggttacttttgacaattctggggagttataaactcaaaaaactaatagtatccagtcacagtgaatttgaaagctggaatagtttgtctttacaatgggtttctgttcactgtcagttatcattatattttggcctttggtggggatgtctgcttgtttttgcaaaagaagatggcagaatttagacttgacagaggagaaatgctcagggtgagattaggtgtagtaatctgctgtttacctccagttatatgtgcaaactcccaagccactaataacttcagttatgcactctaacacagacgaccacctgaaatgcactggtatttatttctgataattaaaaattacaggggagggaagaactagaaaaagaacaactttagaccaaaggtgtctgagaaaaggagaaagggagcttgttcttcccattgc

SEQ ID NO: 296 - DNA, 460 bp, human
  misc_feature (62)..(62): n is a, c, g, or t
taaagatcttgctgtggctctgttatgttctgaggccttgggattagcctcttcctcattanggagctgattttctagtctgtggatcagctatgcctttggacacttctcttttccattgtgccttttgaatgttgtcttctcactcagcatcagcacttcgatctaaatgcagactaggnagttgggaggaggaaccaaagtgaaccatccttcatttattcagtcattcgttcatctgtcaaacacgtatttggacatcaaggttgcagagatgaacaatgcatggatttcatctttgaggagttcaaaacctagtggagagaacacatggtacaatcgtaacacatgaaggacaagtaagtgctgcagtaaaggtactaataacatgttccttggaacagaggaagaaaaaccacgaaaccatggaaattagggaagcctttacagagggtgtgac

SEQ ID NO: 297 - DNA, 415 bp, human
ctcccaatgctgctcagcttgcagtcatgcagggagccaacgtcctcgtaactcagcggaaggggaacttcttcatgggtggttcagatggtggctacaccatctggtgaggaaccaaggccacctttgtgccgggaaagacatcacataccttcagcacttctcacaatgtaactgctttagtcatattaacctgaagttgcagtttagacacatgttgttggggtgtctttctggtgcccaaactttcaggcacttttcaaatttaataaggaaccatgtaatggtagcagtacctccctaaagcattttgaggtaggggaggtatccattcataaaatgaatgtgggtgaagccgccctaaggattttcctttaatttctctggagtaatactgtaccatactggtctttgc

SEQ ID NO: 298 - DNA, 418 bp, human
  misc_feature (223)..(224): n is a, c, g, or t
tcagacacacacaggtcgccagtgacttcacacacacctcatgtgagaaccatgccttttttagtgtgtcctatttcatacctgtacacacttcctcgttttgtaatgagatttacttacacccaaacagatcctgaaagaaagcttcaagttttctcagatgatggatatgttttcactgtattcaataactgacggatgtaaggtgcacgtttcctgatgnntgacgcactgtattccagctggtgatcaagtctgggaacagccgtaacaggtcaaccttgtggagccatcgcgagttagagggtgaaagatggcagaaaaaaaagtcttgtgtgtgagtgtgttttttgagtttgcatcaatcttaatgtctcttcataatacttttataatacattaagcctcttgtctacat

SEQ ID NO: 299 - DNA, 372 bp, human
ggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttgtaatactgatgcacacattacaggagagagctgaacgttctctcagcccagagcagcaaacacatgaaagtcagaagtctctaatatatcatttgtcttttttccagtgaggtaaaataaggcataaatgcaggtaattattcccagctgacc

SEQ ID NO: 300 - DNA, 547 bp, human
tgcagccagcaagctgggactggcaggaaataacccacaaaagaagcaaatgcaatttccaacacaagggggaagggatgcagggggaggcagcgctgcagttgctcaggacacgctcctataggaccaagatggatgcgacccaagacccaggaggcccagctgctcagtgcaactgacaagttaaaaaggtctatgatcttgagggcagacagcagaattcctcttataaagaaaactgtttgggaaaatacgttgagggagagaagaccttgggccaagatgctaaatgggaatgcaaagcttgagctgctctgcaagagaaaataagcaggacagaggatttgctctggacagagatggaagagccgggaacagagaagtgtggggaagagataggaaccagcaggatggcaggggcaaagggctcaagggtgaggaggccagtgggaccccacagagttggggagataaaggaacattggttgctttggtggcacgtaagctccttgtctgtctccagcacccagaatctcattaaagctta

SEQ ID NO: 301 - DNA, 344 bp, human
aggagtggatcctggagcagctcacgcgcctctacgactgccaggaagaggagatcccagaactggagattgacgtggatgagctcctggacatggagagtgacgatgcccgggctgccagggtcaaggagctgctggttgactgttacaaacccacagaggccttcatttctggcctgctggacaagatccggggcatgcagaagctgagcacaccccagaagaagtgagggtccccgacccaggagaacggtggctcccacaggacaatcgctgccccccaacctcgtagcaacagcaataccgggggaccctgcggccaggcctggtgccatgagcagggc

SEQ ID NO: 302 - DNA, 487 bp, human
ttgggattgtgctgactttgggattaacatgagcttctttagcaaccaagcatgaacttgattaagaccagaagtttgggagatgagtcctggcattatgtctaggactaaagcagtggctttgtatagcaagctgagtaaaggttgacatattccaaaacccttctttttaaaatgaaaaaggatggagagaaggatggaaagcctggacttaaacctttagaaaaaacttctggagagaaatcccttttaaacagttacttttgtcattgcctctggtcatttgtctaaataggaatggaaaattaaaagaaaagcaacaatccaatcttttttctaaaaattatgctggggtctcgactaaaactgaatttgaattggaaaattctggtgttggttggagttccatcttgcaagggataatacaaatcctatgatctctatgcccaatatgctgcctcaactctgagctgtctgcaaggcttag

SEQ ID NO: 303 - DNA, 549 bp, human
  misc_feature (225)..(225): n is a, c, g, or t
ggatgggctggaccaggtgggacagattagctgatgcccttgtcacctgccctctgtgcaccctgagagctcacagtaacactgtgtgtgtcaccatataactgcacctcacccccgcacgtgtgcatgactcgcagagaatattccagcaattgtgtacccctgggccagtctctttgaaccctgagggtggccaggatctggagctgcatctctaaggggccnaggctttggggaccattgccaaaggtggactcaggaggaaagacacttaaagacacttttacatgtctagtaattcttgatgttcatcttcagcaccagtggaaacacatgaacttcgatgcaggtccagagaccatggacactcccacgaggctcagctctcaggcaccccctacacttcagttgagggaaaagctcaagtgccttaggcccgtggaccacagtcttggctgagatcaaagggatgagcaacagggacttctgccacagtgacaatggaattgtgttgtgccttacttcagaggtggtctcttctttcttgta

SEQ ID NO: 304 - DNA, 546 bp, human
  misc_feature (104)..(104): n is a, c, g, or t
ttctatgcatccacaccaaaatcctgcagaatgtaagtaagctctgctttataagatgggttcaccttcatcgcagactgaaagtttcagtttttatttttttncagaaagcacgaaaaattatttataatagtctggagaaaaaacacactgtaatatttcaagtgtatgcagtagaatgtactgtaactgagccctttcccacatgtctaggctccaatgtctcctgtaggtccacctaactgtgtgttttcagggacaatgccatccatgtttgtgctgtagacttgctgctgctgaatcctttctggggactttctcatcgggcagggagcagagggcttctcgttcatgcaccctttgcctgaacacccatgtagctgctgtgttgtgtatatattactcttaagaggagtgtgtgtgtctgtgtttgttttaaaagtcacttatttcttacagtgatttcaattgcaccatgacttcttcactaaaaccacaaagtcctgcttaaaactatggaaaacctaacctgattagagccttgac

SEQ ID NO: 305 - DNA, 351 bp, human
ccccacccgctttgaatgtagagacccgtgggcacttttccttttgtggtggggggtgcggaggaggtacccccacccctggcacagccgcctggaatgcaggactgtcactgctgttcgggtgatgacctcgttgccaagctcctcctgtccccttgttctgggggcaggcgctgtgcttctgtgaggtggtttagcttttgctttcgaagtggccagctgcggccaccaggtctcagcacaagagcgcttcctttgcacagaatgagcttcgagctttgttcagactaaatgaatgtatctgggaggggtcgggggcacgagttgattccaagcacatgcctttgctga

SEQ ID NO: 306 - DNA, 508 bp, human
  misc_feature (26)..(26): n is a, c, g, or t
gggagaagatggttgagcacttacgngttgagcacnggaaaaaacttacacnggaaaaaacttacacagaaaaaacatacacttgtgtatgatggaatctgaagccactgaaaatcctacttgaaaagccatgtctaactcacctattgaatttttagttaacagcaactatttttcatacagtaaaatgttactaatttagattaagggagtgctgggggaagacactgatgccgaaactgaacaaatgttttaaaagaaatgcaggtttattatccagcactgagagagttaacaaggactggaaaaataaggctaacaaagaataggatgtgaatgttttcaagggccaaattaatgtatttataggtaaccagagcatatgttgcaaaaagacacattttaagaatcaaaatcaattctccaaaatgttttaaactttactgtatattctcgctagcaacagtaattctgtaaataccttttccccccataaacactgcaaggg

SEQ ID NO: 307 - DNA, 266 bp, human
ggttcaccgccagagtcgattatatctccccatcacacgtctgaggattcagaacttatagcagaagcaaaactcctcaggcagcacaaaggtcggctggaggctaggatgcagattttagaagatcacaataaacagctggagtctcagctccaccgcctccgacagctgctggagcagcctgaatctgattcccgaatcaatggtgtttccccatgggcttctcctcagcattctgcactgagctactcgcttgatccagatgc

SEQ ID NO: 308 - DNA, 383 bp, human
agtgcaagagcccttcgtgggagctgtcccatgtttccatggtccccagtctcccctccacttggtggggtcaccaactactcaccagaagggggcttaccaagaaagccctaaaaagctgttgacttatctgcgcttgttccaactcttatgcccccaacctgccctaccaccaccacgcgctcagcctgatgtgtttacatggtactgtatgtatgggagagcagactgcaccctccagcaacaacagatgaaagccagtgagcctactaaccgtgccatcttgcaaactacactttaaaaaaaactcattgctttgtattgtagtaaccaatatgtgcagtatacgttgaatgtatatgaacatactttcctatttctgt

SEQ ID NO: 309 - DNA, 530 bp, human
  misc_feature (68)..(68): n is a, c, g, or t
gattaaacgactgtgtctttgtcacctctgcttaactttaggagtatccattcctgtgattgtagacntttgttgatattcttcctggaagaatatcattcttttcttgaagggttggtttactagaatattcaaaatcaatcatgaaggcagttactattttgagtctaaaggttttctaaaaattaacctcacatcccttctgttagggtctttcagaatatcttttataaacagaagcatttgaagtcattgcttttgctacatgatttgtgtgtgtgaaggacataccacgtttaaatcattaattgaaaaacatcatataagccccaactttgtttggaggaagagacggaggttgaggtttttccttctgtataagcacctactgacaaaatgtagaggccattcaaccgtcaaacaccatttggttatatcgcagaggagacggatgtgtaaattactgcattgctttttttttcagtttgtataacctctaatctccgtttgcatgatacgctttgttagaa

SEQ ID NO: 310 - DNA, 519 bp, human
  misc_feature (112)..(112): n is a, c, g, or t
gcctaggccacggatgcctcaagagacccggtcagcccagactggaggagtgccccaaaccagtccagtgtccacttgccagaatttcagctccttttttctgtggtgaggncaaaggctagaaataaaatggaatccgcgcccaagtgggacccaagcaggcatctttcggtggagaaacgccccccagtgtccttgacacagctgactgctggcatgagcccaccctctgcctgaagatgcaggaccatttttgccttaaaagttggggaggagggacatgtagattgtgtgcattgtgcatagtcaaggaaatcctagggccacctccagttcatttgtgtgggaacaaggatattttatagatacaaattatttttatgctgtgttgaattaatcaattaggagaggaaggggaaatcacttccttcaaactttttatctgattgtctaaaattctaaccatgcttttaacttattatttttacccagctctgaaggtcattgttcttgcctgtg

SEQ ID NO: 311 - DNA, 540 bp, human
gtgtatctaagctgctttacaattttttttcaatgctgtactactgtctcaagatttaaattttaatgcagagtactttattggtgtgaggcacacaggtaagaagaaatgtcaacattaaatgtatgacttacttggtacaaaaattttttaaaaagggaactaccttgacattgtgtattaaatgtttacctaagactataatctcaagtatgatgtttgtttaacatatacctctcaaaatttatcaccactcaatgacactgcatcaaaattgactataaaactaattcaagaaatatttatatatattttttaatatacaaaaaatatttagcctgatggaatggctttccttttcaaacattattttctaagtttctatacaaatgaaatctttacctctgcatattaatgagccttgccataattactgtagagtggcttttcaaagatattttgttgcactaaaactgtggtagtaaactcagtgaacatgatgtgtggaagagcataattagctggtcaatatttttgtcc

SEQ ID NO: 312 - DNA, 382 bp, human
tttcccttaataccgttgcaagtctgacagcatgagtatctatgacgatattgatgctgacgtgctgcaaaattaccaagaatacaatctggccaacatcatctactactctctgaaggagtccaccactagtgagcagagtgccaggatgacagccatggacaatgccagcaagaatgcttctgagatgattgacaaattgacattgacattcaaccgtacccgccaagctgtcatcacaaaagagttgattgaaattatctctggtgctgcagctctggattaatgaaaatcaagttccatcctcagacaagaggtaaagaaggaaaattcagccagttgattttgtttttagcttactgctgcctttgtccgaagaaac

SEQ ID NO: 313 - DNA, 474 bp, human
gcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctaga

SEQ ID NO: 314 - DNA, 290 bp, human
taggaccttcaagatcctagggggttttgtttggtttttaattgtgaggaataaaaaatcttctgcccacactggcattttaaggtgactgaggtcaaacgttgtttccttaggttgaaatagcagccaaaacattcttcacgcaggggcttgggatatggctgctggcaacacattttgttgtgggctccttaatttaatgataaaatttaagctaaacacaagccaaaaatgaataggtttttttaatttttatttttcactaaacaggcaattgaaatacatggtac

SEQ ID NO: 315 - DNA, 407 bp, human
  misc_feature (47)..(47): n is a, c, g, or t
gcgcctagtcagagagtgggtgaggaggagatcctgatctacatcantgaccatgaggacaaaaacgaagaggcattttgcgtgaaggtcatctaccagtgagggcttgagggtgacgtccttcctgcggcacccagctggggcctgtctgtgcccctcctgccctgcaggctgtcctccccgcctctctgcagcctttcacttcagtgcccacctggctgacctgtgcacttggctgaggaagcagagaccgagcgctggtcattttgtagtacctgcatccagcttagctgctgctgacacccagcaggcctgggttccgtgagcgcgaactccgtggtggtgggtctggctctggtgctgccatctacgcatgtgggaccctcgttatcgctgttgctcaaaat

SEQ ID NO: 316 - DNA, 373 bp, human
ccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatagactccat

SEQ ID NO: 317 - DNA, 526 bp, human
  misc_feature (88)..(88): n is a, c, g, or t
agcagtgagttattacccacaatttaaagcaaattttgaatgaatctttaagttaggtatctaccttcccaccagccccccaaaaaanctctcagtagtttctttcagtgtacaaaatgatgagcatttttctatgatgaggttttaaccattattcagggtggtcttttgtttttaaatctttttttaactaataagatttacggtgtgtattttatacagaaatgcattataaatgtttttaattgtgttctgttttttgcagtctttaagtgccatgccaattgttcttatattctatagaagttcgctcaaaatactcaacaggggaataggcagcggacagtcagaatggttggaattttggctttctaagaaaaactttattttgcataagcatgtggtcagatcattttgtgcatatgcagcctggattggatgttaagtaaatgcttgttcagtgccggtacatttacttaaatctgtttttatttttgtcatgtagaatactactgtggtcatcata

SEQ ID NO: 318 - DNA, 327 bp, human
ttctcatcaatacctgttccactgagctcctgttgcttaccatcaagtcaacagttatcagcactcagacatgcgaatgtcctttttagttcccgtattattacaggtatctgagtctgccataattctgagaagaaaatgacctatatccccataagaactgaaactcagtctaggtccagctgcagatgaggagtcctctctttaattgctaaccatcctgcccattatagctacactcaggagttctcatctgacaagtcagttgtcctgatcttctcttgcagtgtccctgaatggcaagtgatgtaccttctgatgcagtct

SEQ ID NO: 319 - DNA, 474 bp, human
  misc_feature (39)..(39): n is a, c, g, or t
gccttgcatggtgcctgtagggggatttgctttgctgtnccattggggtacanctnctgctcttactctagaccnaaaagtcgggttgagtgactggtggcagggccacnganagagacagcggggagggtggctgaccctggcggncctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacataggtttggtaaaatgagtgctggaaattaactttctcccagtagtcttaggtcatgctcagtgaacttaaactttatccagatatggttttccttcagcctttctattccctttctagccagtgaaagacccgctgcc

SEQ ID NO: 320 - DNA, 586 bp, human
  misc_feature (175)..(175): n is a, c, g, or t
gcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggntcgcccacctacagcatgtcctactcgcagcagggcacccctggcatggctcttggctccatgggttcggtggtcaagtccgaggccagctccagcccccctgtnggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccngcacnggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctca

SEQ ID NO: 321 - DNA, 314 bp, human
  misc_feature (50)..(51): n is a, c, g, or t
gccgtcacatggatggtggtctattaacttgttcaaaaaagtatcagganntgtcaaggcagagaagagagtgtttgcaaaagggggaaagtagnttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcct

SEQ ID NO: 322 - DNA, 147 bp, human
  misc_feature (1)..(1): n is a, c, g, or t
ntcatgctgtcaccctgaatcataatcgctcctttgcacctctaaaaagatgtcccttacccnnnnnnnggngggctcctgagcctctgcgtaaggctgaacgtctcactgactgagctagtcttcttgttgctcgggtgcatttga

SEQ ID NO: 323 - DNA, 510 bp, human
gagctcacagcacaagtgtatcactgtttaatgttacccaacaagagttagtgttaagtgatgatcaagttcccatttcacctgctctacttttgctgcattaattaatgacacccggatgaggagacgtgcgctaacttcattgctcatctgggatagtgcatgagcccattgaattagagctgctcctactagataactgagcagtacacataagtgcatgttatgaaacatgaatcacatagagcagtggagttttaccaagtggtgtgtgtggtttttgttttttactatgcaaagatgggaaatgcacaaacttttcaaagactagtgtctgaagaactttacaaacaatacttgaaccctttctttaaagttatcccatcatgttttatagtcattgttgcttccattgttagtttccattttcaagtgctttgtaattttttaagtgcactacctgaaattttgtttgaaattaataaattcattcgtatcttgttggctgcc

SEQ ID NO: 324 - DNA, 503 bp, human
  misc_feature (169)..(170): n is a, c, g, or t
gacattgcctgtatgatcgggtaccgaccttgcccctggatgaaatggtgctggtccttcttcaccccgctggtctgcatgggcatcttcatcttcaacgttgtgtactacgagccgctggtctacaacaacacctacgtgtacccgtggtggggtgaggccatgggcnnggccttcgccctgtcctccatgctgtgcgtgccgctgcacctcctgggctgcctcctcagggccaagggcaccatggctgagcgctggcagcacctgacccagcccatctggggcctccaccacttggagtaccgagctcaggacgcagatgtcaggggcctgaccaccctgaccccagtgtccgagagcagcaaggtcgtcgtggtggagagtgtcatgtgacaactcagctcacatcaccagctcacctctggtagccatagcagcccctgcttcagccccaccgcacccctccagggggcctgcctttccctgacacttttggggtctgc

SEQ ID NO: 325 - DNA, 465 bp, human
caatatctgacaccactttggactcaagagactcagtaacgtattatcctgtttatttagcttggttttagctgtgttctctctggataacccacttgatgttaggaacattacttctctgcttattccatattaatactgtgttaggtattttaagaagcaagttattaaataagaaaagtcaaagtattaattcttaccttctattatcctatattagcttcaatacatccaaaccaaatggctgttaggtagatttatttttatataagcatgtttattttgatcagatgttttaacttggatttgaaaaaatacatttatgagatgttttataagatgtgtaaatatagaactgtatttattactatagtaaaggttcagtaacattaaggaccatgataatgataataaaccttgtacagtggcatattctttgatttatattgtgtttctctgcccatt

SEQ ID NO: 326 - DNA, 125 bp, human
cctccagatgatctaaatctagttagcaatgtcagcctgtggactgcagctgtttgggacattgtattcctttgtttgtctcttgttgttctgaaggattgtattgatcacaaagctatatgcat

SEQ ID NO: 327 - DNA, 156 bp, human
ggagcatatcaagtaagaggcgtcgccaccaccctcccgagggcccctctgcggatggagccccaggtgcaacagggcccatggtcatctgtggagggacagcccgtgtggaaactgcatttatcttcttgacctgatacccatgcagtaaatgaa

SEQ ID NO: 328 - DNA, 302 bp, human
gaacgggtcgagatgccgacgtgagtgagtgggggcatgcttgggaggcgcaggatggtactggcacatctaacatctacacttctctagctcagcctcacaggccaaagcatcagcaccagaacgcacacccagcccagccccaaagagaaagaagagacagcaaagagccgcagccggtgcttgcacaccgccttgcacatagcagaggctccaggctgactccttcctggtgggaaaggaagatgcctgtcctctccgtggaggaccctgggccctcaccgcaggcagcagtttgcatt

SEQ ID NO: 329 - DNA, 419 bp, human
ggagggcttgccatgtttagaaggagaaacttaagtgtggaatgcattatatgggcaaagaagctatgaagatacatgatacactttgtacaactatcctgcagcccattggttgcttatatttatcgcttggctcaagttctgccctttggagaaatactgagcaagtctttcattctctgtgtgacagccctctgaatatttgaagttgtttgttgtaacttaaggttataacagcccttagttcatttactctgcatttgttcaataaatatttaactgaattcttcaattatttcatctaagatagtttctggaaatttcactctcgatctttctgtggacacaatctattttgtcattgtgtctatatgaatctcttaagtagaaatgagttgtatggtgaatctgtgtagtga

SEQ ID NO: 330 - DNA, 452 bp, human
  misc_feature (235)..(235): n is a, c, g, or t
atatgggcgtctggaccgagagctccagagagccaattctcatttcattgaggagcagcaggcacagcagcagttgatcgtggaacagcaggatgagcagttggagctggtctctggcagcatcggggtgctgaagaacatgtcccagcgcatcggaggggagctggaggaacaggcagttatgttggaagatttctctcacgaattggagagcactcagtcccggctggacaangtgatgaagaaacttgcaaaagtatctcatatgaccagtgatcggcgccaatggtgtgccatagccatcctctttgcagtcctgttggttgtgctcatcctcttcntagtgctgtgannnnncatcctgctcattttgacaaacgcagagtcatgtacccatcactacaatcaagttacagaccatctccataactccaaagaattccacacactcc

SEQ ID NO: 331 - DNA, 263 bp, human
  misc_feature (146)..(146): n is a, c, g, or t
gcagcaggtgaaacaaccttgccagcctccaccccaggaaccatgcatccccaaaaccaaggagccctgccaacccaaggtgcctgagccctgccaccccaaagtgcctgagccctgccagcccaagattccagagccctgccagnccaaggtgcctgagccctgcccttcaacggtcactccagcaccagcccagcagaagaccaagcagaagtaatgtggtccacagccatgcccttgaggagctggccactggatactga

SEQ ID NO: 332 - DNA, 334 bp, human
  misc_feature (32)..(40): n is a, c, g, or t
ctctcctcatgttgcctttcggagaagagggnnnnnnnnnctggccnccagcccccccggccgcnccagggagagctggggaggaaggcntagtgcccccacggcgtccccccctctctataccatctgagcacccattgctcgccatcagatcaacccctgattttacatcatgangtaatcaccactggagcctcacngtnacnaaantattaatntcttgcccccagtgntctatctctgaggctgagcattataagaaaatgacctctgcnccttttcattgcananaattgccaggggcttatttcagaacaacttccacttactttcc

SEQ ID NO: 333 - DNA, 275 bp, human
  misc_feature (85)..(85): n is a, c, g, or t
tctaagaaggatagtcccccctacaacatactgtcatactgctgggttttcatgggtaggaaagcttgtcctgaccccagcagcnaagaggtggcaggtcgctaatgaatatatgctttataatgtccttcttcattgctgagagggcagccttagagctgtggatttctgcatcccccctgagtctgacccatggacacctgtttcattcactttagcatcacagtgaccttngtatgctctgttcagtctgtgtcaggcagtatgcttgtcct

SEQ ID NO: 334 - DNA, 536 bp, human
  misc_feature (152)..(152): n is a, c, g, or t
ttcagctgtctacttggaagaacatggaataagcatacttaacagtgaaaagaatctttaattatgtattatatctgtaatatatttattttgtttaaagaaggctttctaacaatgactgactaaataaagctgtctgctcctgcattganaannaannnnnnnnnnannnnanacccctccccccctnnntttggcaaaggaggggaaaggaaggtttaaaataattgatttaaaatgtcactaagtgtagactgatgactgtatagagatgtgaaatgtataattacacatggaagcaatatgttgctgtgtgctataggttttgttttgttttctacatcttttaaagacttttggaaatttggctgaacaattagaacacaacaggccaactcatactcatttgggatctatttagacaacgttaaccaatatatctatagctttagattatattcgataaaagtaatgggactttttttctttttttgactcgttgaacagtgtcttcgtaatatgttcttagttccctt

SEQ ID NO: 335 - DNA, 431 bp, human
aaccaggagttttaatcgccatatattttaatgtgcttgcatgatgggaatgtgacttttagcactgcatggtgtttgggggcaagatattaatgtggaaatcttagcttaagttttactgtgctctcatcctgtgtcagctacccctctaaactgaaaaagaggcatggctcatttctctcctgctttatgtttttgaggttgacttgtaagataaaaataaaaataagaaaaaaatatatctaggcaaatgacatgaaaaaaaagttgaaatatactactggtgttcatttttgtcctgcagtgttgctttctcaaagaaataaacatgtggctggaagtgtttaatggctgcgttttgatcgtctacaacaaggttacagtgccctctggtggcagtcatcaaaatcgcttctagacttgtttttatt

SEQ ID NO: 336 - DNA, 511 bp, human
ctaccgccttgttttaggagcctgcctcctgtcagtatgaaaccctcacctgaaaaatgccagcctggacaccaaacactgagccccttcaacaggcacattatttccccctgagatccataagggaatttagtttctactattgtagagttctgaaaagaggtaaaatagtagtcctttggtcatcctatttttgctttcaattttgatatttcagactgtaaaaggccttgggggatgatagtacatgtggtagcagtaatttttttgaagcaactgcactgacattcatttgagttttctctcattatcagattctgttccaaacaagtattctgtagatccaaatggattaccagtgtgctacagacttcttattatagaacagcattctattctacatcaaaaatagtttgtgtaagttagttttggttaccatctaaaatatttttaaatgttctttacataaaaatttatgttgtgttttaaaatccttaggggctttatctat

SEQ ID NO: 337 - DNA, 393 bp, human
  misc_feature (76)..(76): n is a, c, g, or t
gttagccaggatgtccacttttttcttaaatgaacatattgtcttataaccagcaaatgaagttttacttttttantcagaaaaaatttcgtcataaagctatatttttatatttttaaaattgatacagttagataccttcagattacctaagttaagtttttgccaaaatgctattttatgccctttttgtaaagttatgatttattttgtaaatatagttccttatagttttctcttgagtgtttggacatgagaatagataagattaaattacaatttacaagatcacttcattaggcacaggaaaagtatttaacaaaattcaacaccctttcacaataaaaaataaaaaataaatctatgaagaacccatggttagcatcatactta

SEQ ID NO: 338 - DNA, 310 bp, human
  misc_feature (45)..(46): n is a, c, g, or t
ctgctgggcggaagggcctgtggacagcccacccaggcggcgggnncttggtgggnccagtggtaatggtgcccctccccnccattccctgtgggtgcagcccacaggctggcgaggaggtgcctctgccctaccctcgggtctggcgagggagngggcagggtgcatccactgtgacagcncctctgggcccacaactccctgaccctggaagctgagcctgctttcctgaccctccgccccaccccctcctccccagaacactcagagggacggtcaacatcagagcacattaaacgctggtgaaacc

SEQ ID NO: 339 - DNA, 259 bp, human
  misc_feature (55)..(58): n is a, c, g, or t
ttgtactgagctgagaccttgccactgcgctccagcctggtgacagagttaagtnnnnaaaaaaaaggcatcttcctaaagnaattgtatttgtgcttacctgtgccaggcactgttctaggtaagcactaagtgggctttaatacagcatattccaatggggaatcccaggaaccaaaagactaattgtccaagtccacaactagaagtggcacntctgcagaaacaagcatcaaattccctgctcaggaagaagcca

SEQ ID NO: 340 - DNA, 356 bp, human
gtaactcaaacccctatgtctggaacatctgtattacatctgaattttttcacctagtcaattcctgcacctctctctcctgtcccttcttccctcccctggcagtcagctactgttaggattttgttcactgtagattaattttgcctattctagaacttcatataacggactcacttatgcagtatgtattctgtaagttccttttactcagcatgtttgtatgttcatcagtgtgtttaagctttgttcctttttatggctgagcagtgttctatgtatgaatgccacattttaacgtttcacctgttaggtgaacaactaactggtgaacaatgggctgtttctagtttggg

SEQ ID NO: 341 - DNA, 276 bp, human
  misc_feature (32)..(35): n is a, c, g, or t
gcataaattaatcagccccagagtgctttaannnnttctcttaaataccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttntaatcttatcttttggcttctgcagatggttgcaatngatccttaaccaataatggtcagtcctcatctctgt

SEQ ID NO: 342 - DNA, 508 bp, human
  misc_feature (146)..(146): n is a, c, g, or t
cagtacctcgcgccaggagaacgcgccgcccgcgccggcctcccaatagccgccgcccgctgcctgcactctaagcgctctccccacgtccgctccgggagccatccccggtcgcaggagtccggggagggccgggaggcgccatngtctctctctgtagcctttcctggtagtcaatttgttgtcccgaggattcatctttttcttccncctnccttctgttttttttnnnncntgtatncattggtcttgcctgagctcttccncaaagcttggaggaacgggggaggncccgggaatgtccctgtctgcacgacctgggactctgcccatgtgcttttgcctgtggaatggagacgcggaccctggatagtggttctatgactctgcgagggacaggcccacgcgtgtggggagaacatctcccttccggggctgccctcaagagcttctgaaaaactaatgactctgctgccttctcctttgtctttgtttcactctgtgtttc

SEQ ID NO: 343 - DNA, 561 bp, human
ggaagacctggagtttggcacagcaggaggggaggtagaagaaggtgcacctccacccccaaccctgcctccagctctgcctccccctgagtctcccccaaaggtgcagccagaacccgaacccgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagactgaggatgggagtgacaaggtgcagcccccaccagagacacctgcagaagaagagatggagacagagacagaggccgaagctctccaggaaaaggagcaggatgacacagctgccatgctggccgacttcatcgattgtccccctgatgatgagaagccaccacctcccacagagcctgactcctagccatcttctgcaccccactctttgtttccaataaagttatgtcctta

SEQ ID NO: 344 - DNA, 408 bp, human
  misc_feature (83)..(83): n is a, c, g, or t
agaaaggtggacgctgggcagctctggggctgctggtggagggagctgaagccacatctctgtagagcgtcccccggtggatnagccaactncaacccttttncngtctttttctgcttgagattcaaattccatggacaaacgnttcaaatgtcccagtttgagtaaaatgtntaanccagcctatggagataagttattttcatttgncaaaactgggttgcttctatcagaaatggggaaaatagatgttggatagcaaaagtccnccatgtccaggccaggcgctgtggctcactnttgtaatcctantcactttgggaggccgaggcaggcggatcgcctgaggtcaggagttcgacaccagcctgaccaatatggtgaaaccccgtctctattcagaataca

SEQ ID NO: 345 - DNA, 496 bp, human
  misc_feature (150)..(151): n is a, c, g, or t
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccgggnntgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagngnntcatgccagggcgactcnggagggcccctggtgtgcaaggtgaatggcacctggctncaggcgggcgtggtcagctgggncgagggctgtgcccagcccaaccggcctggcatctacacccgtgtcacctactacttggactggatcc

SEQ ID NO: 346 - DNA, 251 bp, human
gaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagt

SEQ ID NO: 347 - DNA, 330 bp, human
aagctgttttccaaggcaactgcaccattttacattcccagtaacaacctgtgagcattcaaatttttccgtatcgtcaaccaacatttattattatctttctcattgtggtaaaatgtctattcagattctgacattttagcttttgatataatcactttagggtcttcctcatgtaatatttatatttttccctttttctgtatatccttttttttctctctgaaaaacgtaacactgtcagccagaggaagtaatatattctagggaatgggaaactcatgcttcctgtgagtctttaaactgtccattgtccatcttggcaacatg

SEQ ID NO: 348 - DNA, 447 bp, human
  misc_feature (186)..(186): n is a, c, g, or t
gccttttgggagtgcgtggtggtggtctgggtgtatggagctgaccgcttcacggacgacattgcctgtatgatcgggtaccgaccttgcccctggatgaaatggtgctggtccttcttcaccccgctggtttgcatgggcatcttcatcttcaacgttgtgtactacaagccgctggtctacaanaacaccnacgtgtacccgtggtggggtgaggccatgggctgggccttcgtgctgtcctccatgctgtgcatgccactgcacctcctgggctgcctcctcagggccaagggnaccatggctgagtgctggaagnacctgacccagcccatctggggcctccaccacttggagtaccgagctcaggatgcagatgtcaggggcctgaccaccctgaccccagtgtccgagagcagcaaggtcgtcgtggtggagagtgtcatg

SEQ ID NO: 349 - DNA, 308 bp, human
taaatctgggtgcatagcttgagccagtaatttgggctaaagtgtgtttcggacttagaaatttgtgcaatagtcaactgtctaaagcagtgttgttcaaatagaactataatgtgagacacacaagtattttaaaattttctagtagctaccttaaaaaagtaaaaagaaatgtgaaattaattttaaggacttaacccaatatgttgcaaaatataatttcaacatgtattcaatataaaaattattagatattttaggctgggcacggtgactcacgccagcactttcggagtaatcccagcact

SEQ ID NO: 350 - DNA, 518 bp, human
gagtatttcggcccgtatcgaacttttgttctcagtatcagcccatggtttcaggatcaaagctgtcatgttggagattggtaatggctttcctgtctttgtacagttgaattcctagtcttccttcatccttgccctctgttggcacaggcattatctctgcaattttagaaaatgacaagtagagaatactacattgagaaactaaaccctcttcttggggtcctgatactcattcccatttgtcccagtgctgacaacccaatcttcccaatactttcaggcctgctctacaaaagtacctgttcttgtagaaattttacagtctgccattttgggtgcccaccccaatttttaccttttagtaagttggcatgaaattttggtaaaatctgaaaatcacatttcagaataaaacaattgggcaaaactacctaggctttactcttgagtgtctccttttgatagggattgtttctggaccagtttgtctaagtcctggctcttattggttcata

SEQ ID NO: 351 - DNA, 435 bp, human
ataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctattctgtagtatttttatttggatatttctagtatggcacatgatatgagtcact

SEQ ID NO: 352 - DNA, 149 bp, human
gagcacaagtaaatctgtacagatgatgacgcagagccattcctttagcttcactttcctggaggacttgcaggccaaaattctagggattccatataaaaacaacgacctaagcatgtttgtgcttctgcccaacgacatcgatggcc

SEQ ID NO: 353 - DNA, 514 bp, human
cgactggagtcgttgggtttgggatatttttggtgagtggttggtgaaaagggaaattttttttggaagtgcaaaacatttgtattgttttctttgataatatatttttcccaaaaactaaaaattggttacgtcacaaaaagtattgagagacgaggctggtataatcaactctttcaacccatgttgccacaacttcctgtatgatttgttataatacaatacagattgagatattaacacatgaattttaggttttattagtgttatctgggctggccaccaggtggcataagtggttcaagaaacttgagcttggctgtaatcaaaagcaagatagaacctttccacatcagcaagattattcttatctccacttctctgaatgtttttcatttctttctgaaatagcatgaacataaggctgatctttggtcagttactgagggagtttgtagaaaatagtagagtgagtaagaaggctagagtgaagagtcagtatagtcttgtgttt

SEQ ID NO: 354 - DNA, 470 bp, human
  misc_feature (41)..(41): n is a, c, g, or t
ccatcattctcggcaaactgacgcagggacagaaaaccaancncngcatgttctcactcataggtgggaattgancgatgagaacacatggacncaggaaggggaatatcacacactggggcctgttgtggggtggggggaggggggagggatagcattaagagatatacctaatgttaaatgacgagttaatgggtgcagcacaccaacatggcacatgtatacatatgtaacaaacctgcacgttgtgtacatgtaccctaaaacttaaagtataataaaaaaaaagaaaattaaacaactttaaaaaaagagtaattacagcactgtaataaatgaaccatgcacatgggagtattcatagagaatgataacttcaacctaaatcttgaagtataagtataaatttgccaggttcacctcttgattatttaagtgtgaaagaggatgcatagatcaccttcaagtag

SEQ ID NO: 355 - DNA, 243 bp, human
cccagggcagagggtcagcatctcctgcactgggagcagctccaacatcggggcaggttatgatgtacactggtatctgcaacttccaggaacagcccccaaactcctcatctatggtaacaacaatcgggcctcaggggtccctgaccgattctctggctccaagtctgccacctcagcctccctggccatcactgggctccaggctgaggtagaggctgattattacagccagtcctatga

SEQ ID NO: 356 - DNA, 543 bp, human
gtggaatgtcatccttacttcaaccagagaaaactgctggatttctgcaagtcaaaagacattgttctggttgcctatagtgctctgggatccctccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccgatctctgatgaatattaacatggagggcattgcatgaggtctgccagaaggccctgcgtgtggatggtgacacagaggatggctctatgctggtgaatattaacatggagggcattgcatgaggtctgccagaaggccctgcgttgtggatggtgacac

SEQ ID NO: 357 - DNA, 122 bp, human
tcctccgaagatgtccttgacataggagtagtcacctccagatttggggatggtgaccccgagttcagcatagcagagggctcccacaactgtgatgaagcccgtcacaatccagacgatga

SEQ ID NO: 358 - DNA, 516 bp, human
ccttttgctcagagatacctaaaatatattacagggtaataaaataaatcaactagatcaataagtttggtattgttaattttaaagtaactttattttataattgacatgtgtagtttgacaaattaaatgcaataccaagcttaaaataaaaatacagtgatactgtcccatcattccccagtcttacttttctatcctgggggcaaacacttttgaatctttcagttgcttaatttggcatttatatatctccatagttttatataatatgtaaatatagctattttgatttataaatgttaattattatcaaacttcaagttttagcagaagtgtggtgatgaatcagttttaaatccatctctcctaaaaactgtagataaatatacacacatacttgtgtgggcatgcacacataaaacatgaagagtgcttacagttactacactgattgacttcatcttctggtaagttatagatgaactctttcatgatgccagttctatgtttttc

SEQ ID NO: 359 - DNA, 544 bp, human
ggatctctgtactaaaagcctcccgtagaaatttggttgatcggattactgccctccgatgccttgaagcccaagtcagtacagggggcataattgatcctcttactggcaaaaagtaccgggtggccgaagctttgcatagaggcctggttgatgaggggtttgcccagcagctgcgacagtgtgaattagtaatcacagggattggccatcccatcactaacaaaatgatgtcagtggtggaagctgtgaatgcaaatattataaataaggaaatgggaatccgatgtttggaatttcagtacttgacaggagggttgatagagccacaggttcactctcggttatcaatagaagaggctctccaagtaggtattatagatgtcctcattgccacaaaactcaaagatcaaaagtcatatgtcagaaatataatatgccctcagacaaaaagaaagttgacatataaagaagccttagaaaaagctgattttgatttccacacaggacttaaactgttagaagtatctgagcccctgatgac

SEQ ID NO: 360 - DNA, 509 bp, human
ggaaggtccccattgcaatattcagtgaaatcgcattctttaccaactgatttcctacaaatcatattagccggtttgaattctgaactttttctgacttgatactctgaattaaaccaagtataagtgtcattttcttctaagagaatattagtgtcagcatatttttcttcataaatcttgtgtataaatcctgatacagcctccatcggttcaattccatatgagatgtttttcagctgcattgttcccctgagtcctgaacatacgctgagagtcacaagagaatttggaatacccgcaacatatccattataattgcaatccatctgaactagcagaggtttggaatgacggatgtcattttcgtcataagaattaataacagaagctgaagataaaattgattgctttttgagatggacaaaatatggctttccctatggtaatcatgtaaataacattatgttctggatctgtttttgcatccgatgatgaaatcttctctg

SEQ ID NO: 361 - DNA, 173 bp, human
aaaggaggaactgacactagtgaataatgttttaaaactggctactaaactgctaaaggagttggacagtccttttagattatatgggcttacaatgaatccgctgctttataacatcacccaggttgttatcctgtcagctgtttctggtgttatcagtgacttgcttggat

SEQ ID NO: 362 - DNA, 113 bp, human
gcgctggttcctgcaagtgcaaagagtgcaaatgcacctcctgcaagaagagctgctgctcctgctgccccgtgggctgtagcaagtgtgcccagggctgtgtttgcaaaggg

SEQ ID NO: 363 - DNA, 257 bp, human
agctgcaggtgcaggagtccggctcaggactggtgaagccttcacagacccggtccctcacctgcgatgtctctggtggccccatcagcagtggtggttactcctggagctcgatccggcagccaccagggaagggcctggagtggattggggacataaattatagtgggagcacctactacaacccgtccctcaagagtcgagtcaccatatcagtagacaggtccaagaaccacttctccctgaaggtgaggtct

SEQ ID NO: 364 - DNA, 271 bp, human
gggaccccagggcagagggtcaccatctcttgttctggaagcagctccaacatcggggaagaaattactgtaaactggtaccagcagcttccaggaacagcccccaaactcctcatctatagtaataatcagcggccctcaggggtccctgaccgattctctggctccaagtctggcacctcagcctccctggccatcactgggctccagtctgaggatgaggctgattattactggcagtcctatgacaggagtctgagtggtgtggtat

SEQ ID NO: 365 - DNA, 492 bp, human
gctctgaatctatacctaatgctcttaattattggcttgtttcatttttttcctccagtttttaacaagatcacataactggcttatttttaacagctttgtcaaactacaatttacatgccgtaaaatgtacacactgtaattttataattcattgacttttagtaaattttctagcgttatgcatcgccacaatccagttttagaatatttccatgaccctaagaagtttcctcatgtctattaatattcccaatcctaggcaccactgagttgttttctgtctttataagtttttctttctacatcttatataaatggaatcataatacatgtagtattttgtgtctggcgtcttgcacttagcatggtgttcttgaggttcatctgttgtagtatgtattgatacttaggatttttttattgccgaatactattccattgcatggaaaagacctattttatttctaggttcaccagttgagggacatt

SEQ ID NO: 366 - DNA, 523 bp, human
gtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgg

SEQ ID NO: 367 - DNA, 427 bp, human
gttttgctaactggctatctggaatacctgatcaagcataactatggcaaagataaagcagcaaccaagaaaccagttgtgaacataattactccgtctcatgtagaggagcgggggtgccagctaacaataacattttctgttccaaacgaagatgttttccaagaactagaaaaaagaggagtggtttgtgacaagcggaatccaaatggcattcgagtggctccagttcctctctataattctttccatgatgtttataaatttaccaatctgctcacttctatacttgactctgcagaaacaaaaaattagcagtgttttctagaacaacttaagcaaattatactgaaagctgctgtggttatttcagtattattcgatttttaattattgaaagtatgtcaccattgaccacatgtaacta

SEQ ID NO: 368 - DNA, 503 bp, human
gaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcagcaaccagggagactgcacctgatggaaaagatatatgactgcttcatgacattcctaaactatcttttttttattccacatctacgtttttggtggagtccctttttatcatccttaa

SEQ ID NO: 369 - DNA, 270 bp, human
gctgccaggtgccagatgtgacatccagatgacccagtctccatcctccctgtctgcatctgtaggagacagagtcaccatcacttgccgggcgagtcagggcattagcaataatttaaattggtatcagcagaaaccagggaaaactcctaagctcctgatctatgctgcacccagtctgcaaagtgggattccctctcggttcagtgacagtggatctggggcagattacactctcaccatccgcagcctgcagcctgaagattttgc

SEQ ID NO: 370 - DNA, 452 bp, human
gatgaaatctcactgctaatgctcagagatcttttttcactgtaagaggtaacctttaacaatatgggtattacctttgtctcttcataccggttttatgacaaaggtctattgaatttatttgtttgtaagtttctactcccatcaaagcagctttctaagttattgccttggttattatggatgatagttatagcccttataatgccttaactaaggaagaaaagatgttattctgagtttgttttaatacatatatgaacatatagttttattcaattaaaccaaagaagaggtcagcagggagatactaacctttggaaatgattagctggctctgttttttggttaaataagagtctttaatcctttctccatcaagagttacttaccaagggcaggggaagggggatatagaggtcacaaggaaataaaaatcatctttcatcttt

SEQ ID NO: 371 - DNA, 320 bp, human
  misc_feature (48)..(48): n is a, c, g, or t
ccagggcccgtggggatgtgtgggttctgtgggtgggtgcctgggaanggaagtggcacaaaggggtgaaggcccaagcaataccaaggagccgtgcacacacattccaggagcttctctgagtcttncctctgggacatgctnctgggnctgggacctggggtcttatggcctggtggaggtgccaattacantgtananggctacgtgggctccctgtaatatcaatgccattcttcgcagaaatagaaacaataaccctaaaatttatatggaaccacaaaaggcccagaatagccaaaccaccctgagcaaaaatt

SEQ ID NO: 372 - DNA, 410 bp, human
  misc_feature (31)..(31): n is a, c, g, or t
gtaagtggaagctgaccaggttgtcaggctngnnttncnatgttactctgcatgactcccctttaaacgtcagtctttgttctgcaatgccntcttgtcacagggtcatctactgcttatttgtggcactgttttgtcttctgtttatgtntatttcacagcttgtcagagtctatacaactcagcagaaagaacacggcttgcctgctccctctcttgaagattgattgcaatgggagtggtttctctgtttctttgtatagtcgaacagatatttacttcttccaagaagatacaattcagaggaatttttatggcaaaagttagtggagacggggtggatgaaatctgttgggagagtatcactgttctgatggncggccttgagcaccacactgtaatactagacc

SEQ ID NO: 373 - DNA, 512 bp, human
ccaccagtgtatcaccagagtcctggacaggctccatacccgggaccccagcagccttcataccccttccctcagcccccacagcagtcttactatccacagcagtaatatgtctgctcagcagctcagctgattcagatcagagggaaagaaataccaaccctgcaataagtgtactaaactctacgctctggttaatgtaatgtactctcctggactgaatgcagtgtataatttctgtctacagctagaagctgtgccccagttccacatttgattacacatgtgagatttgctgctgttgcagtataaacactaggtataataggatttgaaattgcattacagttcataaaaattgaaaatgagaaattaaacctgcaagtgaaacatttgaaacgattatactttctacataagacatggttgggacatcagatacttacaaagatggtttaagtatggatactagagaaaattaagttttctttctctttggtttattgatttgg

SEQ ID NO: 374 - DNA, 524 bp, human
aactgtactttgtaccctcacatacaaagggatcaaatttgacctggtgttattttagccccaaatttatgacattacacaatattaaaatgtaaatgtttctttacccaaactacttctagatattctagtatttgcttctggtggaattaaatgacggtaaaattggctaattatttgaatgaatgaatggatggatgttttgcatgctcaatttctaggtcctttgtctagaaaggaaatttgcctcagttgaattagtgaaatatttctgtcgttgatattaaaagtgacttctgagtacagttaagttcctcctatttgccactgggctgttggttagaagcataggtaactgattaagtaggtatgatactgcatttgaaataagtggacacaaactatcctttctccaccatggactcaatctgagaacaacagcattcatttccattcatttccatactggcttttgattatatgcagattcctagtagcatgccttacctacagcactatgtgca

SEQ ID NO: 375 - DNA, 411 bp, human
tgtggtacggctgcacaacttccaccagctctctgcaccccagccctgtttcaccttcagccatcccaacagagatcctatgattgacaacaaccgctattgcaccttggaatttcctgtggaggtgaacacagtactacatggctttgcggtctactttgagactgtgctttatcaggacatcactctgagtatccgtccagagactcactctcctgggatgttctcatggtttcccatcctcttccctattaagcagcccataacggtacgtgaaggccaaaccatctgtgtgcgtttctggcgatgcagcaattccaagaaggtgtggtatgagtgggctgtgacagcaccagtctgttctgctattcataaccccacaggccgctcatataccattggcctctagcc

SEQ ID NO: 376 - DNA, 351 bp, human
gaaccagtgtatgtcaacaggtgcatttgagataactttaaatgatgtacctgtgtggtctaagctggaatctggtcaccttccatccatgcaacaacttgttcaaattcttgacaatgaaatgaagctcaatgtgcatatggattcaatcccacaccatcgatcatagcaccacctatcagcactgaaaactcttttgcattaagggatcattgcaagagcagcgtgactgacattatgaaggcctgtactgaagacagcaagctgttagtacagaccagatgctttcttggcaggctcgttgtacctcttggaaaacctcaatgcaagatagtgtttcagtgctggcat

SEQ ID NO: 377 - DNA, 426 bp, human
tcacctgccacagagcaacccaggttaaatacagcccatgcacaaagccacaggccaaagcctatggaattgtttttaatcatcaaatttaaccattttcataactggttcctggaggtgtgcagtgcccccttgcctcttcaaacctacagcttctctttgccatttgtggatttcacatcactccacacagaaacattacagcctggcatccccagtctttgccttcttccagctgcctcgacacagcactgtggcctgtccctattgcccaggcacgccatttccaagggcaggaaggggcagtgtcctgaagcccatcttttctgtgactgtcttaggtgatgtgtagccccctccacctttccactcaacaacctcccacccctgtcctgctgcatggtccggagtctgggacctactttg

SEQ ID NO: 378 - DNA, 539 bp, human
gacaatcgcactttataccacctgcgattactggttcaggataagtttgaggtgctgaattacacaagcattcctatctttctcccggaagtcaccattggagctcatcagactgaccgtgtcttacatcagttcagagagctgccgggccgcaagtacagccctgggtacaacaccgaggtgggagacaagtggatctggctgaagtgaacgggccgccttctgctccagctgcatcacagtgatggccaagctgcatcagccgcactctcctggacgccatatagctttaagatcgggggagggtaaataatgcaaaaattgcacagtggaagaaggggtctcacaaaaagcaatccatcctgtagtataggtaatggagttgggggaagcagcttccattctggatgtttggaaccctttagctttgttttggaatggcccaccattctcactggaaaacagtggtctgctgtgaaaggccagctctcggcagcccctgtggtttcagcgtgccgctctgtgtcattcaggttgtg

SEQ ID NO: 379 - DNA, 508 bp, human
gaggaggggaagcctgtattcaggatgatagagggagcagagacttactgtctctcttccacattataaatgaactgcctttccaggtccaacatgctgtcaggttgcttcagctcggtgtgaatgtaagtgtggggatcaaacacacacacctatacacatacatattaatatgcagaggattttaaataattgcaaatgtcttgtggctgttttatttctacaaaattctgtaagatgtataccagaaaacataataaagataagctcagttcttcattgggaaaaaatatttttaaagaaaatttagaagtttttgctgaattaaagtatcttcaattttaattactggatttggaagttgaaatgccatccttcaatatcactggtctcaaattcattgatagaattttttcttaaactaataagtggggtaagtataatctcctaaattggtacccactggctgctcctaaactggtttccagtcatgtcctccagtatgtga

SEQ ID NO: 380 - DNA, 549 bp, human
aaagagctgggtctggctgagcaccagctgcgcttcacctgccgcgtgcacctgcatgacacacgcaaggagcaggagacggcattgcgcgtctacagccacctcaagagcgtcctgaaggaccactgtgtgcagcacctcccagacggctctgtgactgtggagtccgtcctcctccaggccgccgccccttctgaggacccaggcaccaaggtgctgctggtctcctggacctaccaggacgaggagctggggagcttctcacatctctgctgaagaagggcctcccccaggcccccagctgaggccggcaactcacccagccgccacctctgccctctcccagccggacagaccctgggcctgcacttcaggactgtgggtgccctgggtgaacagaccctgcaggtcccatccctggggacggaggccttgtgtcacctgcctgcccaggcagctgtttgcagctgaagaaacaaactggtctccaggctgtcttgcctttattcctggttagggcaggtggtcctagacagcagtttccagtaa

SEQ ID NO: 381 - DNA, 489 bp, human
gtctccatcgggaagggggtgaccgtgggtgagggtgtgcggctccgggagagcatcgtcctccatggagccactttgcaggagcacacgtgtgttctgcattgcatcgtgggctgggggagcaccgtgggacgctgggcccgcgtggaggtacccccagtgaccctaaccccaacgatccccgagcccgcatggacagtgagagcctcttcaaggacgggaagctgctgcctgctatcaccatcctgggctgccgagtccggatccctgccgaggtgctcatcctgaactcgattgttctgccacacaaggagctgagccgaagcttcaccaaccagatcatcctctgagtagggctgccagaaggcccccagctcctacccactccccttgaggactgctgcctgcttggccagcctctgtccagaaaggaccagagaaagccaggctggatcgtcacatgccggggagcaatgtggatggcctggg

SEQ ID NO: 382 - DNA, 524 bp, human
gtcttgtttcactgatcttagctttaaacacaagagaagtctttaaaaagcctgcattgtgtattaaacaccaggtaatatgtgcaaaaccgagggctccagtaacaccttctaacctgtgaattggcagaaaagggtagcggtatcatgtatattaaaattggctaatattaagttattgcagataccacattcattatgctgcagtactgtacatatttttcttagaaattagctatttgtgcatatcagtatttgtaactttaacacattgttatgtgagaaatgttactggggaaatagatcagccacttttaaggtgctgtcatatatcttggaatgaatgacctaaaatcattttaaccattgctactggaaagtaacagagtcaaaattggaaggttttattcattcttgaatttttcctttctaaagagctcttctatttatacatgcctaaattcttttaaaatgtagagggatacctgtctgcataataaagctgatcatgttttgctacagtt

SEQ ID NO: 383 - DNA, 516 bp, human
tggcaaggagcggcccgaagtggtcctggactcagatgctgaggatttggaggacctgtcagaggaatcagctgatgagtctgctggtgcctatgcctacaaacccatcggcgccagctctgtagatgtgcgcatgatcgactttgcacacaccacctgcaggctgtatggcgaggacaccgtggtgcatgagggccaggatgctggctatatcttcgggctccagagcctgatagacattgtcacagagataagtgaggagagtggggagtgagcttgctagctgctccagtacttgagagcgactctgtgtcccaggcacagctgtgctgcggcagccacgaagccagtatggccaggtggtggctcctgcagcctggaaaaaaatgtgcagtggcctctgagagccccagcctgagccagtcccagctgtgcttggagtctttatttattttaactatttcttcgaacattccacatttgatgatgatacctctttcttccctgagtgtatat

SEQ ID NO: 384 - DNA, 523 bp, human
aaatgttattttccctgtgatagaggatgacttcccagtttcaccaaagtctgtttatatcaacacacacaaaatggaataattctgagtcactaggcaatcaatctactgtggttttactatgtaaggtgaaaattaactggaacgatgtttgtttgctatacttacatagtcaaactttacaagccatgaaattaattgcactctttgtatttgttgttaaatgcctaagaagttttctaaaaattttgtaaaggcactgtcagagaatctggagttgaatgattattccagatactgtataacctgcataactttttgtctttaagtcgtgtttgtaaaagaagtaattgctagaaacatttgataatgtacaaagtagtctataatgactgttcagtacatttttaatatttttttggttatatccaactttttgtaaatatactggaagcttgataataaaatgtatttcctatcaccatacttttccatgtgaaaacctgagcctatttctagtata

SEQ ID NO: 385 - DNA, 186 bp, human
gaagatgaatcggttggggaagaaaggggaggcggatagacacgtgtttgatatgaagcccaagcacttgctgtctgggaagaggaaagctggtaaaaaggacaggagatagtatccgtttggttggcgtggcttcgctagagtgttgctgtttatttcctggtttggtacagtatggtttcatga

SEQ ID NO: 386 - DNA, 265 bp, human
gcacttgttgctttgctgaaaactatggttaaccctatataggtgtgggaaatttttgtcactgcataatattacaaatattttgagtagacagtgtttccacatttaatggagtatcagttgcttcagattttcagaactgggaagatttactggtgtaactgggttgtttttgatggagaaaaaccttattttcttttgtaagagctgggagcaaacacgtttatgagtgtgtcggaatcccgtgcttaaaatacgctcttaa

SEQ ID NO: 387 - DNA, 494 bp, human
gagacaattcgttatcttgcagcttcggtgtttacttgcctggcaatagcattgggattttatagattctggaagtagtattaatgctcatcctgctgtggctgttggcttcttagaacaccaaaccgggagagatttactttgaacattgtcagttgcagcaaaaatttactacacaagattattcgaagtgtatacggactaaaagaggaagtgttttagaatgagaagagatactgtgtctttattgtgtgtgtgtgagtgcaggtgtgtgtctttattatattgaaaagctgtcactcagacctggtttgagatagaagagcattttgtccttttgatagttaatagaaattgaaccagagttttcttatgtttgcttgaacagttgtgtaaatcatacaggattttgtgggtattggttgaatatttgtaaaccattccctagcctacatatttattactgaattaactttcctgataaccattgcata

SEQ ID NO: 388 - DNA, 454 bp, human
tggaggagtgggtcaccattgggcgcctcttcagcttcctgtaccagagcagccctgaccaggttatagatgtggctcccgagcttctgcgtatctgcagcctcattctggctgacaacaagatcccaccagacaccaaggccgcactgttgctgctcctgacgttcctggccaaacagcacaccgacagctttcaagcagctctgggctcactgcctgttgacaaggctcaggagctccaggctgtactgggcctctcctagactgcaggctgcagccagtccagagagaatagagcctgcccaggccttaagaccacctctcagcccagttcagttctgccttaccaaagattttgagactcatacccatttggagccagccccacttgctgccttacagggctgtccctgaggctggatctgttacaaatgagtcatgacatcatactgta

SEQ ID NO: 389 - DNA, 530 bp, human
gaaatatgtcacagtggcattgcagttgtctgttagctttgggttgcagtgctagatattgttttaaattattttcattttaaacaagatgccttctaagctattgagcttattaaaaataattttacatgtttacttagttggagcaaaaataagtctattttaacgaatagctttgtttttgctatgctaatgtctagaaaggcatacgatgctactattatgctctgttttaaaggttttacctacccttgtaaaaactataatcttaaatggttttatttgctgtttactacttatacatactactactataaaactattttttcctaaatggtacaaatttataaactatcatttttcacttacggtatttgtaaatactactactacaaaaatcagctttccgagaaagaaataatcatttatttatgatattgaaaatttctacagtaaacactcaaaaccaagcaaaaaacatttgtaagatacacggtatctatttggagcaacggtttttgtaactaatg

SEQ ID NO: 390 - DNA, 355 bp, human
tttgttggatgtgggccttagcctccaggtccagactactactctatgttctccagaagggtgctaagtcacctactgaagagagaaccaactgactttcctattgactcatcaggaaccagtcctcagtctggtcaagttgtttcttatttgtgagcagttcaggctatctcctgatggggatgaggccaaggctttcttatcttttggttgtctctgcttaatggaggagcctggcctaggatggaggcctggcttagatctttcattccacctcaggaatgaggttgtgatctttcctgtcctgaccctctctgaattatgtttcaatagtactcttgattgtctgccatgt

SEQ ID NO: 391 - DNA, 427 bp, human
ggtccccgtctacaaaggagtctgcaaatgcttctgccggtccaagggccatggcttcattaccccagctgatggcggccccgacatcttcctgcacatctctgatgtggaaggggagtatgtcccagtggaaggcgacgaggtcacctataaaatgtgctccatcccacccaagaatgagaagctgcaggccgtggaggtcgtcatcactcacctggcaccaggcaccaagcatgagacctggtctggacatgtcatcagctcctaggagatggtggaagcaccccttgtcctgtgcttgaggagactttgcggggaggaggcagcagacactggagatgacattcttccacacgagacggggcttcagccggggatggtccctctcaagtatctcctggaggaaggggtatggggggcaggtgtg

SEQ ID NO: 392 - DNA, 501 bp, human
ttaaaagtaagtcgcagccgctcctcccgcagccacttcagcagcatcttagattttaagcctcacgtgcgcagctggttcatgaactattggctgcatcctgcttaggtgcccaccaagaaggtttttacctacttaacaaaaaagaaagaagccaaagtgattagaaagaaatgaaatctctttttgggttctgtctactgaaatttaatatctcagtgaacagactaaaaggaatttagaatcctaacaacttaccagatttctcctgttttaaatatactgggactttaaaggttatatgtccggtcaccgtatgttttaagtcggtgttaatgctaacagtgttgaaaacaatatttcatgagatctaattgtggttgcccctataggtagcaggaaagtaaagttgcatttccctctcgcacattctacacccaagtgcctaaaagatctcattgtaagtgggtagtgttaccggaagccattgtgttcacacgg

SEQ ID NO: 393 - DNA, 414 bp, human
tgatctgtgccattctagacatccctgtctacaagagtcggatccagtccctccatctgctcttttccctctactcagaattcaagaactcacagcattttaaagctctcgctgaaggcaagaaagcattcactccttcatccaattccacctcccaagctggagacatggagacattaaccttcagctgagacacttcccaagctgctgtttcaaggctgagctggcccctctgccccagctgagatggacagatcgttgtcagctacttgatgtccttgcccatgccacagcttggctcaggggcagtgcatgtcctgctgccctctctgccagagggcacagaacatgtttgtttaatgaacctgcctgcctcagattgctgtccccggggagttaatgcatctacaccac

SEQ ID NO: 394 - DNA, 370 bp, human
tggtgtatgctgtgctttcctcagcagtatggctctgacatctcttagatgtcccaacttcagctgttgggagatggtgatattttcaaccctacttcctaaacatctgtctggggttcctttagtcttgaatgtcttatgctcaattatttggtgttgagcctctcttccacaagagctcctccatgtttggatagcagttgaagaggttgtgtgggtgggctgttgggagtgaggatggagtgttcagtgcccatttctcattttacattttaaagtcgttcctccaacatagtgtgtattggtctgaagggggtggtgggatgccaaagcctgctcaagttatggacattgtggccaccatgtggct

SEQ ID NO: 395 - DNA, 493 bp, human
ggcagcccacaagtttctcgtggggagatggaggcagagcccagggtaggggacagagctgctggggcctttccttgcctgggaatctgtcccaggaagagcttccccactcccatcccccaaattggaaaaaccgtacattcaagcctgtttggccctgaaattcttaagaatctggttaagaattaactcactaatgtcaaaagtcaaaacctcctaggggttgtcctgggagtcaggttcacgggtacagaagatgaatctcagatgtcactcaacctgagccgtcattctctgtggcagggctgccctgggtttctcttactcaatccctggagtgtaagcatttggattgtgtcacagattacctttttaccttttctttctttttttttctttttttcaatatcagtgcccacaccttactgagtattgagttttagagctttcgcttgatgtgcttgaccaagagacttcttttgtatccttttct

SEQ ID NO: 396 - DNA, 554 bp, human
gagacagcaacagccgtagcaaaagcagctgctgctcctgctatgagggtgtatatattttttacccaaagctctggaattgtacatttattttttaaaactcaaagagggaaagagccttgtatcatatgtgaacattgtatcataggtaatgttgtacagacccttttatacagtgatctgtcttgttcctgcagcaaaaatcctctatggacataggaggtgctgtgtcccatgccttcttgccctgacagtgtcccatgggcccccttctgctccctgccccctccctgctactgctgatgcactgtcctctccctgcagcccctggcttcccagccttcctcctgaccccttccaacagccttggaactccagctgccaccaccctctgggtcggacactgggacccactggcccagtcttggctgctgcttacccctagccttgatgcctgcccagggacccccagccccctcccgttgccctgcagctttaacagagtgaaccatgtgtattgtacaggcgcggttgtcattgcagaaaccgctggg

SEQ ID NO: 397 - DNA, 501 bp, human
gaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttt

SEQ ID NO: 398 - DNA, 461 bp, human
tgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatgttctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaa

SEQ ID NO: 399 - DNA, 427 bp, human
gtatgggtcatttcaaagagggcttatgaggctgtgaaacccagagctcttaacgctgtgaccaaagatggaagttctctataggaagccatagcactcctaatgtttggtgctatgttttcctgaggagatataaaacgtaataatccatgattgttgccatgtgagagttttaaaggttaatcaaaatttctcttcttcagggcaaacttgaagataaatcttttgactccagctctttagaggatctaaagtgaccttgatggacagtggaagaaatcacaacatggaattcctcgaataacaatttattgactttaaataattttgtctaatgctacatatacacaattaaaaaacctttacactatttctagaaagtcagcatgtatttttggctcgaagtttctctagtgttttctgtgga

SEQ ID NO: 400 - DNA, 496 bp, human
gaaagtggggctgatccaacaattgaaactgactctggatataattctatggatctagctgtagccctaggctatagaagtgttcaacaggttattgagtcacatttgttgaagctgcttcaaaatatcaaggagtagacacagtcatcagaaaatgtctgcccttttgtttacttcttggtccttataaatgatagttttgtttacttataaatttttacctcagttgcaatatttactggtttttagtaggttttaataaatatttctctgagtaattcactggtttataataaatgtaatactctttttataactatgttttactgtatatttaaaattataaattaatgttttcgtggcatgtaaatttttatggtacagatagttatcatcagtctttgtatcaagtgctgtaatttgacattttcagaaattattctaccctagtcatcttcactcgtgtattaagtcattcactttatatagggtttgc

SEQ ID NO: 401 - DNA, 532 bp, human
gagacagacttggcaagggaccccctggttctgagccagtagctgccatctggaaattcctcttttagcctctccttagaggtgaatgtgaatgaagcctcccaggcacccgctgaatttctgaggccttgcttaaagctcagaagtggtttaggcatttggaaaatctggttcacatcataaagaacttgatttgaaatgttttctatagaaacaagtgctaagtgtaccgtattatacttgatgttggtcatttctcagtcctatttctcagttctattattttagaacctagtcagttctttaagattataactggtcctacattaaaataatgcttctcgatgtcagattttacctgtttgctgctgagaacatctctgcctaatttaccaaagccagaccttcagttcaacatgcttccttagcttttcatagttgtctgacatttccatgaaaacaaaggaaccaactttgttttaaccaaactttgtttggttacagttttcaggggagcgtttcttccatgaca

SEQ ID NO: 402 - DNA, 394 bp, human
ggggcggcggaagcgagtagagtttgtgacatttgtgccagcccctccagcccagtcacctgaggagcctgtaggggcccctgctgtgcagtccatccttgtggcaggcgaggaggacatccgctgggtgtgtgaggacatggggctgaaggaccctgaggagcttcgcaactacatggagaggatccggggcagctcctgaccctccacagccacctggtcagccaccagctggggcaacgagggtggaggtcccactgagcctctcgcctgcccccgccactcgtctggtgcttgttgatccaagtcccctgcctggtcccccacaaggactcccatccaggccccctctgccctgccccttgtcatggaccatggtcgtgaggaagggctc

SEQ ID NO: 403 - DNA, 554 bp, human
tcaggagcaccagttctatccctgctaagtgtcagagatggattcattgctagccaaggtgatggaagctgttttattgtccagcaagacttagactatgtcactgagctcactggggctgactgtgaccctgtgtacaaggtagccacatgggagaagcagatctacacatgctgtcgagacggtcttgtacgacgctaccagctttctgacctctgacttcttggaaagagcagtcccggttagtgaaaaggtttgaccctgatcaacaatgagcagaaacatcatcagtccttcccaaggaccatggcgtttaatgtcttgggcaccccttggaaatcacagaaagtcagctgtactggccgtgtggaactctcatcccaagacctactttgaactgagtaagaaggtcattgtgcccactgcatttgttccaacttctccttgtataaactcaccccagcaacacagggcaaggatatagatgcttttagtttgttcttaaaccagttttgttaaatgtttacaaggacctcagtactaaagcctgttct

SEQ ID NO: 404 - DNA, 435 bp, human
gatcccccaaagtcagacacagagcaagagtccccttgggtacacccctctgcccacagcctcagcatttcttggagcagcaaagggcctcaattcctgtaagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcatt

SEQ ID NO: 405 - DNA, 552 bp, human
gatccccattgagttaatgctctgccttgaaaattatttttctgatccttgttagtgataacattttttttctactgaaggtcagaggataggaaacaagtatttctcttctggtatacatgtaatgtattctgtaaaaaagtattcatattggcaattttagttaggcataatattgtggttgtaatttttaaaacttagtgttttgtctgattaaagcaggcactgatcagggtatctcctaagaggtaattcacttcttattcctttccaataattattacattctaaattttcatctatgagaaataacaaacaagaagggaatagaattaaattggggtataatctaatcttcattgtttaaatggtttgccttctcaccattgaagccatttttttatagcctcagaaagaggaaataatgcctccaccattttctacctggtgacttgaaaattgaacttttaagttaggaagaagttagagtcagggaacttgtataccactatctatgcagcattgttatagtctgattatttctgtgttttg

SEQ ID NO: 406 - DNA, 479 bp, human
ccaggttgtaccaaggtccctgagccaggctgtaccaaggtccctgagccaggttgtaccaaggtccctgagccaggctacaccaaggtccctgaaccaggcagcatcaaggtccctgaccaaggcttcatcaagtttcctgagccaggtgccatcaaagttcctgagcaaggatacaccaaagttcctgtgccaggctacacaaagctaccagagccatgtccttcaacggtcactccaggcccagctcagcagaagaccaagcagaagtaatttggtgcacagacaagcccttgagaagccaaccaccagatgctggacaccctcttcccatctgtttctgtgtcttaattgtctgtagaccttgtaatcagcacattgtcaccccaagccatagtctctctcttatttgtatcctaaaaatacgtactataaagcttttgttcacacacactctgaagaatcctgtaagcccctga

SEQ ID NO: 407 - DNA, 517 bp, human
tgctgcctcatcgaggacggcgagcgctgcgtccggcccgcgggcaacgcctccttcagcaagagggtccagaagagcatctcgcagaagaaactcaagctggacatcgacaagagcgtaaggcacctatatatctgtgattttcacaaaaatttcatccagagtgtccgaaataaaaggaagaggaagacaagtgacgatggcggagattctcccgagcacgacactgacattcctgaggttgatctgttccagctgcaggtgaacaccctacgacgttataaacgacactacaagttgcagaccagaccaggcttcaataaggcccagttagcagaaactgtgagtcgacacttcaggaacatacctgtgaatgaaaaagagacccttgcctacttcatctacatggtgaagagtaacaagagtagactggaccagaaatcggagggtggcaagcagcttgagtgaggatgaagcacatcttaaaggaatgaagtgtaatgcttgatgcacaggt

SEQ ID NO: 408 - DNA, 524 bp, human
accacaggactttgattttaagccaaatccatctccatccctttactgtcaatcttctgtcccagtagtttagcctttgtggcttaggttatgatgcgcctccttctgtgcgaccaatgagacgacttcagcatctttttaaaataatctaagcatcattgaagcagtaacacaaaaaaaaggttcagtattttctttttagtataacttacatcctttcaaataagtctttgccctcatgaagaatccctagaggaagataaggaaaataagtattttccagttttgcttgacagtttctaaacaaacaaaaataaactcaatgaaaggaaagatgtttctttttagctgagatgacagattgcttctctgtattaaatagtctagaagttaaggggatggtcacatttaccatgtattgtgttattagcagttaaattttatgaatatgtttgtaaaattgttgttttatatttcatgtcaaattgaaaagtttatttcttcactattgtacctgtggaaat

SEQ ID NO: 409 - DNA, 494 bp, human
caacaaaacccatgagcggcagccccccagtcctggatggtggtaaagaatcctcaagatcaaacccacgcagtgctgagagcttggcctgattctagggctggggctggagaaactgctagagatgatgccgatagccagtgtgatccccctgccctgatggtcaagggcagagtgcagactggaaccctcccctccccaaagattcagacctgtggggctgagtgggctcatagtgtccccaagtcctgagaggctggtgtctggcttcagcctccagcttctcaggttctgatgcagtcagctgagttccctgcctattcttgcaagcactaggaggagggtggtgggttgctgggaacagcaccgagcgccctccccacccagattcacagagcacactccccggggggatactttaatccggaggccgtgacgcctggctccgccccgagacgagctgaatccaaatacggatctaggcttgagcttgg

SEQ ID NO: 410 - DNA, 442 bp, human
ctgtgaagtctgaggtctgggggcttcagaccagagtggaacaatttgtacacaaggtggtccgagacattctgctgattggacatagacaggcttttgcatgggttgatgagtggtatgacatgacaatggatgaagtccgagaatttgaacgagccactcaggaagccaccaacaagaaaatcggcattttcccacctgcaatttctatctccagcatccccctgctgccttcttccgtccgcagtgcgccttctagtgctccatccacccctctctccacagacgcacccgaatttctgtccgttcccaaagatcggccccggaaaaagtctgccccagaaactctcacacttccagaccctgagaaaaaagccaccctgaatttacccggcatgcactcttcagataagccatgtcggcccaaatctgagtaacttta

SEQ ID NO: 411 - DNA, 431 bp, human
gtgacttcaggtaccagcttaaagagcactagggatggggaacgaatgccaaatcagactccacctagagcaccaggaaacagcttgtaccctggtagggaaatggtgttgctgaaaggggaggctgagccagtgcgagactgaacttgtgcagccttagccaagacaaagcagtgtttttcagcagacggctgatgggacaggaattgaagaagagaattgactcgtatgaacaggacagggtgaaaatgctgggaattataatgggaaacaaaactatctatgttcatattttgtaatatttcatttgttaagtttatatctggatataatgttctttttaaacaagtataatcatatcgtcggaggttaagattatgaaattttagaatctctattcaagatgatgttcactccaaatacactacaga

SEQ ID NO: 412 - DNA, 488 bp, human
cctcctacagccttaagtcccaggcccatgtctgcctgtccaagggctcaagactttctaactgggatgtggtagagggactgaaggtacctttgggggcaacagcaccctagtttcattctcaactctagccctgcacactcacctgtggcacggaatgaaaacagagcttcccgtgcaaaaagggtcacgcctcccacccccgccccctccctgcacctcctgtcctctcccagttcattcctggaaccagccaggccaggcaaccagtggcccccaaaggcaggcaggatcctcaggccccagccgcgggaggctggaagggctggcagatcgcttccctcatccacctccaccggtccaggtctttgctgctgtccccagacctcctgtgacaccacgccagatcacagggcaccaggccagagatagtcttctttttgtcctttctggcctctggctagtcagtttttcatagccttacagta

SEQ ID NO: 413 - DNA, 577 bp, human
cgctgatgacaccactgcgggagcgcttcctgcagccgctgatggccctgctgtaccctgactgtggcgggggccggctcgacagccaccgggcctttgtggtcaaatacgcaccgggccaggacctggagctgggctgccactatgataatgccgagctcaccctcaatgtggccttgggcaaggtcttcacagggggcgccctgtattttgggggcctcttccaggcacccacagccctgacggagcccctggaggtggagcacgtggtgggccagggtgtcctccaccgtggcggccagctgcatggagcccggcccttgggcactggtgagcgttggaaccttgtcgtctggctccgagcctctgctgtgcgcaacagcctctgtcccatgtgctgccgtgagcccgacctggtggacgatgagggcttcggtgatggcttcacccgagaggagcccgccacggtggatgtatgtgcgctcacctgagcttgcttgggcccagtgtgggggtggcaggcaggtgagggctccgttgccttggtctgggggcagaaataaaatccccgcagcct

SEQ ID NO: 414 - DNA, 528 bp, human
taagcaatgaacccatctcgaaggaggagtttgtaatacacaccatatttcctcctaatggaatgaatctgtacaaaaacaatcacagtgaaagcagtagtaaccgaagctacagagacagtggtattccagactcagatcactcacactcatgatgctgaaggactcacagcagacttgtgttttgggttttttaaacctaagggaggtgatggtaggaaccctgttctactgcaaaacactggaaaaagagactgaaaaaaagcaatgtactgtacatttgccatataatttatatttaagaactttttattaaaagtttcaaatttcaggttactgctgcgattgatgtagtggagatgcctgaacacaattctatattttagtattttttagtaatttgtactgtattttccttgcaaatattggagttataaaccatttactttgtgttctactgagtaagatgacttgttgactgtgaaagtgaattttcttgctgtgtcgaacaatcaggactgcattcat

SEQ ID NO: 415 - DNA, 508 bp, human
aacactaagacattatttcactatttatagcttttccttttttttatttgagaaggagtcttgctctgttgcccacattggagtgcagtggcacgatctcagctcattgcagcctccgccacctgggttcaagcaattctcctacctcagcttcccgagtagctgggattataggcacccaccaccacacctggctaatttttgtatttttagtggagacggggtttcaccatgttggccaggctggtctggaactcctgagctcaagtgatctgcccatctcagcctcccaaagtgctgggattacaggtatgagccacaacgcccagccatcatttatctttttcactcattttttcatgagtatgcagaggagttttcaagaggctatctggtgtgatattgtaataggctgaatgcaaaagcagatacaagattccagctttcgtctgtcatcagacattgaagagatttggagaaatgtaaagcagcagtactcttctcacta

SEQ ID NO: 416 - DNA, 479 bp, human
gtagagctcatgaactctgtgctgtcttctggtaacatatcatcagtgtttgtattcatggtgtggcacatggatccatggcattgggtaaatctggtggtttttacacatggtcagaatgtgttcaaatacatctcatgatggagacagtaaccaaggtaatgttttgtttcagcattttaaaaagactccacttaacatttatctcagaatcatgagcctctctctagttgacaattgtcattgttcccccagccccaaattgaaccatacatttattttccaaaacagaaattgtttctgtaaacatatccgaccttcaaaagaagtgtgaggttggatttttcccccttttatttgtcctaagattccaggaggctggcctcccagcccagatagctcagccacctcacctgcctatcattcctgcattcctactttgagggtctcatcatgcccactgggtgttgttcctaaga

SEQ ID NO: 417 - DNA, 332 bp, human
gcaggtgaggagcaggctccacagactctcctggggcccagaggagctggtgctgacctagctagggatgcaagagtgagcaagcagcacccccacctgctggcttggcctcaaggtgcctccacccctgccctccccttcatcccagggggtctgcctgagaatggagaaggagaagctacaaagtgggcatccaagccgggttctggctgcagaagttctgcctctgcctggggtcttggccacattggagaaaaacaggctcaaagtggggctgggacctggtgggtgaacctgagctctcccaggagacaacttagctgccagtcacc

SEQ ID NO: 418 - DNA, 495 bp, human
tcgcctccagtcataacagtcaaacttctatgcacaaaaaagccaaggactccaaaaattcctcctcagctgttcatttaaatagcaaaacagctgaagacaaaaaagtggcaccaaaagagcctgtggaagagacagacaggcccagcccagagcccagcaatgaagctgccaagccaagaaatacagagaaggacagtaatgctgagaacatgaattctcaacctgagaaaacagttactactcccacagaaaaaaaggagagtgtatctcctgagaataacgaagaaggtggaaatgataaccaagacaatgaaaatcctggaaatacaaattgccatgatgttgtactagtacagtgaattatcagaaaacccagaagccagtttaggctagagaggaaaaaactactgtatcatttatcctaaagaacagagatgaggtcaatttcaaattttagccatctgtttgtgatttctgtcataagcattttgt

SEQ ID NO: 419 - DNA, 324 bp, human
ctcagcccctgtcaacagtggggaccccaccaccaccatcctggagtgattccaactcaactcaaaggacacccagagctgccatctggtatctgccagtttttccaaatgacctgtaccctacccagtaccctgctccccctttcccataattcatgacatcaaaacaccagcttttcaccttttccttgagactcaggaggaccaaagcagcagccttttgctttttcttttttcttccctccccttatcaagggttgaaggaagggagccatccttactgttcagagacagcaactccctcccgtaactcaggctgagaag

SEQ ID NO: 420 - DNA, 522 bp, human
gaatgttgcactctgatctcttaacaaattgttacgttcaaagtttaaagtgatatattaacaaagtcacctagttatacaaacaattgtcagagaattctggatttggagggtattggggttatatgattctttcttagataatggcctctactaaataactcaagatctttctggaatgtcttctggcaggcaggtgccactgtcagcttttctccaaaaagcagccaacatcagcctcccctgtcaactcaacagttttgtatctcatattatatggactttatatgaaaatgaatattttacagtttgcacagtattattttacagaaaaggaatcagagaatctacaacatagggccccagaacaacagtttcactttgtggcttttaattattctagaattttaactgcatctcatttttctagcatggtgagaactaatatgtaactcctttgattgaaggagctcttttgtccgtacctatcagaatgttttcttgacacttccatgttggctc

SEQ ID NO: 421 - DNA, 431 bp, human
tcttttttttgagacagcctcgctctgtcacccaggctggagtacagtggcataatctcggctcactgcaagccctgcctcctgggttcatgccattctcctgcctcagcctccctaatagctgggactacaggcaaccaccaccactcctaccttttttgtatttttagtagagatggggtttcaccctgttagccaggaagttctcaatctcctgacctcgtgatctgcccgccttggcctctcaaagtgttgggattgcaggcgtgagccactgctcctggcttgaattttcttataatcccatgaaaccatcatcagagtcacaataatgaatttatctaatctatcacctttgtattcacctggagcctttataaagtttttctctatgcagacaaccattgatttactctgctgtataatacttt

SEQ ID NO: 422 - DNA, 483 bp, human
tcccttggtgtaggaacaacggaggagtttctcctctgctaactattcatatatgtaactgtaacaaaagtgtactatgttatgcacacattacaaataatataaggggaagttttattagcttagtaggaaattgttattattaaggtttaaaaatgagaacaggtgtgagttttccaaaatacttaaaaataatagtgtcaaaaattcaggggcagttaaggagtcatggatggaactagaggtcactatattaagtgacataagccagaaacagacaaacattgcatgttctcaattatttgcgggatctaaaagtcaaaacaattgaactcatggatatagagagtagaaggatggttactagtggctgggaaaaggggtgtgcgaggggaactggggatgcttaatgtgtaaaaaactatgtagttagaaagtataaataagacctagtatttgatagcacaaccgggtgagtatagt

SEQ ID NO: 423 - DNA, 500 bp, human
gccaagcttgttggctcatgcctatagtcccagcactttgggaggccaaggtgcaaggcgggcagatcatttgaggtcagaaattcgagaccagcgtggtcaacatggtgtaaccccatctctactaaaaattaccaaaaaatgaaccaggcatggtagtgcatgcctctaatcccagctactcaggaggctaaggcaggagaatcacttgaacctggagacagaggttgcagtgagctgagatcacgccactgcattccagcctgtgcgacagaatgagacactgtctcaagaaaaaataaaagtgggaaaatcttccaactgtcctctagagtgtgaatatatgaaaggagtcagagtggggtgaaagtctataaatgtaagacatttgggaaagccttcacacagccttttgagcacacatgagaatgtatactggagagaaaccctataaatattaagaatgtgggaaattcttcatcctagttcttttgttgttg

SEQ ID NO: 424 - DNA, 540 bp, human
gaggacaatatccatgactgctcaaaacttaaaagtcctttgggggtcaaatggcatacggcagtcacctatgtgaacagctgcttgtttgtggccgtgctggtgattctgatcggatgttacatagccatatccaggtacatccacaaatccagcaggcaattcataagtcagtcaagccgaaagcgaaaacataaccagagcatcagggttgttgtggctgtgttttttacctgctttctaccatatcacttgtgcagaattccttttacttttagtcacttagacaggcttttagatgaatctgcacaaaaaatcctatattactgcaaagaaattacacttttcttgtctgcgtgtaatgtttgcctggatccaataatttactttttcatgtgtaggtcattttcaagaaggctgttcaaaaaatcaaatatcagaaccaggagtgaaagcatcagatcactgcaaagtgtgagaagatcggaagttcgcatatattatgattacactgatgtgtaggccttttattgtttgttg

SEQ ID NO: 425 - DNA, 502 bp, human
tcaacttcctggtacaatggccaccctttctctggtgaatgaggcacagtatctgctgatcaacacatccagtattttggaacttcaccggcaactaaacaccagtgatgagaatggaaaggaggaattattctcactgaaggatctcagcttgcgttttcgtgccaatattattatcaatggaaaaagggcttttgaagaagagaaatgggatgagatttcaattggctctttgcgtttccaggttttggggccttgtcacagatgccagatgatttgcatcgaccagcaaactgggcaacgaaaccagcatgttttccaaaaactttctgagagtcgtgaaacaaaggtgaactttggcatgtacctgatgcatgcatcattggatttatcctccccatgtttcctgtctgtaggatctcaggtgctccctgtgttgaaagagaatgtggaaggtcatgatttacctgcatctgagaaacaccaggatgttacctcctaa

SEQ ID NO: 426 - DNA, 234 bp, human
ggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgct

SEQ ID NO: 427 - DNA, 385 bp, human
aaaacacctgaatgactctaagactgatatgtattttcaagtctaagctgtcttacagaagatcttttataaatgtttccttataaatatctcaccattacaacaaattgttttaactgtttttctattagctctagctgcatatttgatgtaaatgacaattactgaaaaaatgtcagaaaaaacattttcagtactaacattaaagtgccatatgtaaaaaagaaaaatgtgatttgtataactaaataacacacaaacatcaagaggctatttatacaaataatttatttccactagggaaagtgcattactggtgaaggtattatcaatttattctacttgcttataatgttacagtgaatgttctggcttactctgcctc

SEQ ID NO: 428 - DNA, 437 bp, human
ccagttcaggccgtggagggacgtgatgctgggctgtgtttactaaacccacgggttttcagcctcttaagcccagctccgatctccaattagttgagagcgctgggttgactaacctctggtatctgagcacagacagagggtgctgtgggtctgctgggtggcagaaatggttccttccggcttggcgttctctcctggccactcttcctgctgcctctgactactcagccttgttttcggtgtgtaggccccagctgcccactggaactgccggctaatgcttgctctcccaagatctttaactcctcctggctgcacctgggtagggatggtggcatcgatgcccctctgtctgctgaaggacctgttgctgcttctgtcttttcacccctccttggctgatgacccagagccctctgatgatggcattctcc

SEQ ID NO: 429 - DNA, 551 bp, human
gcaagtctcgggatcactcagatgcagccaagaaacacaggcatgaaaggggacatcatagggacaggcgtgaacgatctcgctcctttgagaggtcccataaaagcaagcaccatggtggcagtcgctcaggacatggcaggcacaggcgctgactttgtcttcctttgagcctgcatcagttcttggttttgcctatctaccagtgtgatgtatggactcaatcaaaaacattaaacgcaaaactgattaggatttgatttcttgaaaccctctaggtctctagaacactgaggacagtttcttttgaaaagaactatgttaatttttttgcacattaaaatgccctagcagtatctaattaaaaaccatggtcaggttcaattgtactttattatagttgtgtattgtttattgctataagaactggagcgtgaattctgtaaaaatgtatcttatttttatacagataaaattgcagacactgttctatttaagtggttatttgtttaaatgatggtgaatactttcttaacactggtttgtctgca

SEQ ID NO: 430 - DNA, 380 bp, human
gctcctttctgatcttcactaagaacaaaagaagcagcaacttgcaagggcggcctttcccactggtccatctggttttctctccaggggtcttgcaaaattcctgacgagataagcagttatgtgacctcacgtgcaaagccaccaacagccactcagaaaagacgcaccagcccagaagtgcagaactgcagtcactgcacgttttcatctctagggaccagaaccaaacccaccctttctacttccaagacttattttcacatgtggggaggttaatctaggaatgactcgtttaaggcctattttcatgatttctttgtagcatttggtgcttgacgtattattgtcctttgattccaaataatatgtttccttcc

SEQ ID NO: 431 - DNA, 513 bp, human
ttgaagctgcttggcttgctcttaggacaagatatggcattcgccctgaaaatgtgattatatatggccaaagtatagggacagtaccgtctgtggatcttgctgctcgatatgagagtgctgctgttattcttcattctcctctgacttcgggaatgcgagttgcctttcctgataccaaggagacctactgttttgatgcattcccaaacattgacaaaatctctaagataacctctccagtattaataattcatgggactgaagatgaagtcattgacttttcacatggcctcgcattgtttgaacgttgccaaagacctgtggagcctctctgggttgaaggagcaggtcacaatgatgtggaactttatggacagtatcttgaaaggttgaaacagtttgtgtcacaggaactggtgcagaaacataaagaagggaagtgattttctcaaggtctacagttttcaatgacggtactatgtattccattatcttctctttaccaaactt

SEQ ID NO: 432 - DNA, 527 bp, human
gggattttctacccagagacctgcatgttctctccaacgaccagaatattctacccaaatgtcaggaccaagattttctaccaaaatatcagaaagtacactttaaggagccatactctgatatgacagatgagaaagggagagaagacttttctctggcagactatcagtgtttgcctcccaaatcccaggaccaggatgacatcaaaaatcagcaacctgcatcttttatgagagaagaaagagtgagagaggaattgcctctggactatcatcaatatgttgtacctaaaatccaggaccaagactcccctagagaacagaacaagcatatcaaactaccctcatcttttgagaaatgggagattgcaagaggaaatactcctggagtgccattggcttatgataggtatcaatcaggattgagcactgaattccaagctccactggcatttcagtctgacgtggataaagaagaagataagaaagagcgtcaaaagcagtacctgagacatagacgacttttc

SEQ ID NO: 433 - DNA, 548 bp, human
atgtcttatcaacagtagcaatgtaagcagccctgccagccacctcctgtgtgccgcctgccaaagcgtccagagccatgtccacccctgaagtgccctgagccctgcccaccaccaaagtgtccacagccctgcccacctcagcagtgccagcagaaatatcctctggtgacaccttccccaccctgccagccaaagtgtccacccaagagcaagtaacagcttcggaattcatcaggaccacgaaaggatagggataattggctcacctcgttccacagctccacctgcatcttctcatcaaagccatccagggatacacagggagcttctttccccttagcctgtgatctgcccatgatgatccccgacagcaaaatgtttcctttctgaggctgccatgctgccactgtccaggtggagactgagcaaaggaagtcctcagctgtaccggcctttcagagcttctctttgggtgccatcaaagaattattttgatgtcttctgtctctgtctgtcacctggcatgagcttctactatctgtgca

SEQ ID NO: 434 - DNA, 503 bp, human
ggcacaggtccccagatgagaagtctgctaccctcatttctcatctttttactaaactcagaggcagtgacagcagtcagggacagacatacatttctcataccttccccacatctgagagatgacagggaaaactgcaaagctcggtgctccctttggagattttttaatccttttttattccataagaagtcgtttttagggagaacgggaattcagacaagctgcatttcagaaatgctgtcataatggtttttaacaccttttactcttcttactggtgctattttgtagaataaggaacaacgttgacaagttttgtggggctttttatacactttttaaaatctcaaacttctatttttatgtttaacgttttcattaaaatttttttgtaactggagccacgacgtaacaaatatggggaaaaaactgtgccttgtttcaacagtttttgctaatttttaggctgaaagatgacggatgcctagagtttaccttat

SEQ ID NO: 435 - DNA, 313 bp, human
agcccaccagcaggagcttggagtttggggagtggggatgagtccgtcaagcacaactgttctctgagtggaaccaaagaagcaaggagctaggacccccagtcctgccccccaggagcacaagcagggtcccctcagtcaaggcagtgggatgggcggctgaggaacggggcaggcaaggtcactgctcagtcacgtccacgggggacgagccgtgggttctgctgagtaggtggagctcattgctttctccaagcttggaactgttttgaaagataacacagagggaaagggagagccacctggtacttgt

SEQ ID NO: 436 - DNA, 510 bp, human
tttgccttagcacacgttttatggagtacttgttatactaggtttgatttgaaactggtgcttgtcgcagaactgtcagagcatgaggagcgctcctcctgtgggtggacgcattcacgcactcccaggttgcacctgctgctggcggtgagcagggggttcagcagcttgaccgatgccccccgagggggctctccccagcttaaactttgttgtttaaatttgttaactttttatattaatgactattgaaagtggtaataaaaatttatattataggcttcaatgttttcatgaatgttacccaaaaagctgtgttttctttggtcagaggtcaaaatttatgaaaaacaaaatgctgtatgaatggaaatcattttgcaattgagtgacacttcattgtaattcacagtgtaaatttaatccaaactgaaattttgtttcaactgaatttgtaattaactctgaatttgtttttaatcattagtaatatttcagttgggtatcttt

SEQ ID NO: 437 - DNA, 514 bp, human
cactagttcaatggaccctaatgacatgatgagagaaatccgaaaagtgttagatgcaaataactgtgattatgagcaaaaagagagatttttgcttttctgtgtccatggagacgctagacaggatagcctcgtgcagtgggagatggaagtctgcaagttgccacgactgtcacttaatggggttcgcttcaagcgaatatctgggacatctattgcctttaagaacattgcatcaaaaatagcaaatgagcttaagctgtaaagaagtccaaatttacaggttcagggaagatacatacatatatgaggtacagtttttgaatgtactggtaatgcctaatgtggtctgcctgtgaatctccccatgtagaatttgcccttaatgcaataaggttatacatagttatgaactgtaaaattaaagtcagtatgaactataataaatatctgtagcttaaaaagtaggttcacatgtacaggtaagtatattgtgtatttctgttcattttct

SEQ ID NO: 438 - DNA, 508 bp, human
attcagggcagtgctccgaagctgtgtgctcacctggttggctcatcaaacctggcaaccctgtggcctgtctgccggagctgactggatccactcatcaattcttcgtccccactactaagactgggcatgttgtgctggcccaggcgtggggctaccaagcaaaggcttggcatataccaaaggccagctgcatgcccatcagtctggtctttttcctctgcggtcatgttggctttcatgctggatcaaatgttttactttcccagactggtggcatctgagttccccatcctaccactctcaccccactttcctgccccacctaaaccctcgttttagtaatttgtagtgactgttcccttccttctgttgcagggaaccaggaggaaagggaaagatgttgccatatttcctactctttaggcatggactctcctttccctttgttagtgtcctgggttcccatggactcagggatttgttggctaaggtttctctgtgcata

SEQ ID NO: 439 - DNA, 420 bp, human
aaggagctagttttcctgagcctgtgtgtgcaccaggcactggggctgatgttccagcctcgtggcgtgggagcaggagagctccgtcctgcatttctgtggtccgtggccctgtttctgtgcgcctgcgcctcggagagcaagtgaaggagcatcatgggttttgttttctcccttaaccctctgaaagacagtttgatactaacaaaaaacgcagcagaggggatccaacgtcagagcttttagaattacttttttaagcagctgtcttctggctgggtgcggtggctcatgcctgtgatcccagcactttgggaggcgggtggatcacctaaggtcaggagttcaagaccagcctggccacatggtgcaaccccgtcttagtaaaaatacaaaattggcctggcgtgagagcgggca

SEQ ID NO: 440 - DNA, 413 bp, human
gcatctgctgttcctgtatcagccagaggctgttcaggtcttcctgcagcagtcagggctgcaagccctggaaaggcatcaggaagaggcccagctccaggatcgtgtgtatgctctccagcagacagctcttcaagggtgatcttgtttctcaatgtcactcattcccctctctcttaacatcaagcttgtttgtccagtagagcctttggagatttaggaccataatgaggtctcatgttctctgctcccacacctaagccaagacctttgggtcccagctcctccccttccactcagcactatccaggcaggaggaccaaaagggactcagtgtggtctacttactctggggccctagaatccctgcccccccgccacccttcatgtttgcttcagcagctggtagcttt

SEQ ID NO: 441 - DNA, 531 bp, human
gtgcacctctaatatatgccttacaaactccagaggccatattcaaaacagggtcttctcagtgtatgcaaggggctgcagcccctcttctcttcctccccaggttgaacaatacggacagttttcacacatatctacctgtataaccctctgtacctctcataactggtcaacgactgtaacaggttacatcaggtgtttttctacatactttttacacagattctatgcgattaatgtaatttaattcaatgcatcattttattgtactagttcttaggcttgtccttatttttttctaagtgattgtggtttttctcgtggtttttattgtaaaaaatgaaaggctgttgatgcttattctctgtaactaagaattttaccttttgggggaaaaaagcattgctatgaactaatggaattggaacttcatttactcattgtaaatacactattgtgcaaaaaaagttttcactcaattgaattgctagtgttaactgaattttgtctagacaccatttctgttgatga

SEQ ID NO: 442 - DNA, 405 bp, human
aggctctccaggacatgtttgcactacttggggagtttggagaagaccagatctatgaagcccaccaacaaggccgaggggctctggaggcattgctatgtgggggaccccagggggcctgctcagagaaggtgtcagccacaagagaagagctctagtcctggactctaccctcctctgaaagaagctggggcttgctctgacggtctccactcccgtctgcaggcagccaggagggcaggaagcccttgctctgtgctgccatcctgcctccctcctccagcctcagggcactcgggcctgggtgggagtcaacgccttcccctctggactcaaataaaacccagtgacctcacttcttttctctgcaaaaggtgcttgtggggctgggagtgcagacattgg

SEQ ID NO: 443 - DNA, 325 bp, human
gccactatcttgaagaaggtgcacagtgccctgctcctctttcctatgtccccagaattctcttagggttctcagatgccatgactttcaaggagagagtacggaaccacatcatgcacttggaggaacatttattttgccagtatttttccaaaaatgccctagaaatagcctctgaaattctccaaacacctgtcacagcatatgatctctacagccacacatcaatttggttgttgcgaacagactttgttttggactatcccaaacccgtgatgcccaatatgatcttcattggtggtatcaactgccatcagggaaagcc

SEQ ID NO: 444 - DNA, 177 bp, human
agacttaatggagcatttagttccccatcctgaattcaagatgctgagtgttcgtaacattcctcacatgtctgagaattcattggcatacaccacatttacttgggctggcctcctcaagcatttgagacagatgctcatttctaatgcaaagatggaagaaggtattgataggca

SEQ ID NO: 445 - DNA, 395 bp, human
  misc_feature (86)..(86): n is a, c, g, or t
aagtttggaagacaggcttgccgaaattgaggacatgattaaaattgcagtgaagtttgaaatgtttttagcaaaatctaattttngccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatga

SEQ ID NO: 446 - DNA, 468 bp, human
gggcctaaagcccagtgccaagaaaagacgccggtttctcattaaagcccctgagggtcctccaagaaaagataagaatttgccaaatgtgattatcaatgagaagcgcaacatccacgcagctgctcatcaggtacgagtgcttccatatccatttacccaccattggcaatttgaaaggaccatccagacccccataggatccacatggaacacccagagggctttccaaaagctgactactcccaaggtcgtcaccaagccaggccatatcattaaccccataaaagcagaagacgtgggctaccggtcttcctcaaggtcggacctgtctgtcatacagaggaatccaaaacgaatcaccacacgtcacaaaaaacagctgaagaaatgctctgtagattgagttgctggaggagtgacagccaggagccctgacttcacttcctttggtccagttttactctg

SEQ ID NO: 447 - DNA, 405 bp, human
acgcagctgctcatcaggtacgagtgcttccatatccatttacccaccattggcaatttgaaaggaccatccagacccccataggatccacatggaacacccagagggctttccaaaagctgactactcccaaggtcgtcaccaagccaggccatatcattaaccccataaaagcagaagacgtgggctaccggtcttcctcaaggtcggacctgtctgtcatacagaggaatccaaaacgaatcaccacacgtcacaaaaaacagctgaagaaatgctctgtagattgagttgctggaggagtgacagccaggagccctgacttcacttcctttggtccagttttactctgatacagggtggattccaaaactggctcagtacattgcatgtagttaagccaca

SEQ ID NO: 448 - DNA, 380 bp, human
aggtattggcatactccacacatctgtaccattcttgagtgatcgcttaggaatgaatgtgatttgaactcattcatgttgagagggtgtcaaattgagaaccaggtagatccccaccacctacagtaaaaaggaccctaaagtaaattggttgaagaaattagatcccaaagattcttggtgaattttgaagtcttcatcagtatatccatattaaaacgagatgacagaagccaaagtaattatgggctgacaggacaactggatcagtttcattaaaaagggcaaacttgaagataaatcttttgactccagctctttagaggatctaaagtgaccttgatggacagtggaagaaatcacaacatggaattcctcga

SEQ ID NO: 449 - DNA, 513 bp, human
tttactgccttgtgacgttggtcttcttttactcatctgcctcattttgggcctggttggccctgggctttagtctggcagtgtatggggccagctaccactctatgagctcgatggcacgagcagcgttctctgaggatggggccctgatggatggtggcatggacctcaacatggagcagggcatggcagagcaccttaaggatgtgatcctactgacagccatcgtgcaggtgctcagctgcttctctctctatgtctggtccttctggcttctggctccaggccgggccctttacctcctgtgggtgaatgtgctgggcccctggttcactgcagacagtggcaccccagcaccagagcacaatgagaaacggcagcgccgacaggagcggcggcagatgaagcggttatagccattgacattgtggccacaggccactggccctgggtggctctgtcagggtgcacagcccctcatgcctggagcaatgagggtctagtccaggggcc

SEQ ID NO: 450 - DNA, 503 bp, human
atgcatcattggctacacttccatttttgtctactgttgttactgacaagctttttgtaattgatgctttgtattcagataatataagcaaggaaaactgtgttttcagaagctcactgattggcatagtttgtggtgttttctatcccagttctttggcttttactaaaaatggacgcctggcaaccaagtatcataccgttccactgccaccaaaaggaagggttttaatccattggatgacgctttgtcaaacacaaatgaaattaattgcgattcctctagtctttcagattatgtttggaatattaaatggctataccattatgcaagtatttgaaagagacacttgagaaaactatacatgaagagtaaccaaaaaaatgaatggttgctaacttagcaaaatgaagtttctataaagaggactcaggcattgctgaaagagttaaaagtaactgtgaacaaataatttgttctgtgccttttgcctggtatatagc

SEQ ID NO: 451 - DNA, 219 bp, human
ggaaatggatcgaggcaggggtcgcctttgtgaccagaagtttcccaagaccaaggacaagtcccgggatggtgggcgaggccggggccggggccgcccagggaagagagtggcctgagcccaagccgcaccggagcagcggctggattgaacgccccagattggggcccgagatgtggcccttggtttcctttcataaaggagttgtgtccccagccc

SEQ ID NO: 452 - DNA, 251 bp, human
gttgtttttggaaatcgtcagtgagaagtccagatactctctccatcagaacatggacaagatcaaggttccgacgcagatcatctgggggaaacaagaccaggtgctggatgtgtctggggcagacaatgttggccaagtcaattgccaactgccaggtggagcttctggaaaactgtgggcactcagtagtgatggaaaaaacccaggaagacagccaagctcataatcgactttttagcttctgtgca

SEQ ID NO: 453 - DNA, 531 bp, human
  misc_feature (50)..(50): n is a, c, g, or t
gggtgcataccatattcttggttcttaaaatctatcacttttcaccttanacttgatgtgtgaaaactataaaaacaatgtgtgaaacccanggggttctaaaatacaagcatagattttatcagggtgntttgtcaaagcaggttattcagtgattcctccccaccattcttaagaacgttaaataatgctgttgtgttagctctgagtagaaaggaaaaagtaaaacctctgtttggaggtaatattgggttgaattctgactgcccctttctagctggacctttaacaaatcacccaatcttttttgtgtttctctaaagtcatttatacattaaatgtaattatagcaactgtggggttctgttgagaattaagagctaacactatatatgtaaagtttccagtactagtcccagaatttagaatatgctcaacacaaagtaaacagcattatataagtttatatttttgtgagttataaagtactttgatatattctcattaaatctgtaaatcacctctataagt

SEQ ID NO: 454 - DNA, 549 bp, human
  misc_feature (90)..(90): n is a, c, g, or t
aatccaaccctgtcagaatcatgctgttctctttgctgacactgtgaccctgggtcgggacagaccagcagcaatctgtctttagaatcnctttccttcctccccttttgcccccgtggnnnncccngncatcctgaaagccagcaaagcctccagcatcttttccatcctgaggtgcctcccagtggcctggcttgtcggagcaagtttcatcagccctagggaaaacacggccctcctgggaacctccttacctggagtaaccggacaccttagacggaggtgcctgagggtggggtgggatttgcagggtcattatcagaacatgaggataacttccttgcccctgctctgtagccacctccttggcaccggcctctatttgtcataaggcggcgtgggcgaggcctgacacaggccagccttggcacgaggggggccaggggttctgagaagcgctgccctgtgagagccangctggccttcgtctccatctctggttgacgggctgtccgtgtgcctcctgtgtgtctgcagacaagtcttgct

SEQ ID NO: 455 - DNA, 358 bp, human
ccaggatccatttaggtaccacttgatataaaaaggatatccataatgaatattttatactgcatcctttacattagccactaaatacgttattgcttgatgaagacctttcacagaatcctatggattgcagcatttcacttggctacttcatacccatgccttaaagaggggcagtttctcaaaagcagaaacatgccgccagttctcaagttttcctcctaactccatttgaatgtaagggcagctggcccccaatgtggggaggtccgaacattttctgaattcccattttcttgttcgcggctaaatgacagtttctgtcattacttagattccgatctttcccaaaggtgtt

SEQ ID NO: 456 - DNA, 402 bp, human
aaagtcagtccattttcaagttttggtcttcagagacaaaagaacgtcccagccacctgattttgatggtgaggtaactctaagttgaattcaggctagtgttgcagtatagctttggcatgttcatgagtgagcacccagaatgtgttgaaccaacccccacccctaactactgactatgactgcagtgggtttttatggggaaaaaaagtgtgaaaagcaaaaagaaaggaacagagattttttatcacctttattgtaagacagtccatttatgaattgagtataaacacatacaaagtaacaagagattcctaagaaacgcaaatccttgagtttcacgcacttcatgttcaaccatttgctgtaatccagaggcagcctgtgaatcattctcatgcc

SEQ ID NO: 457 - DNA, 491 bp, human
  misc_feature (332)..(332): n is a, c, g, or t
cccaggcctgtcactttgagaggggcaaaactgagaggggcttttcctagagaaagagaacaaggagcttgccaggcttcatgtagccgacacacgtctcaggattttaagtccacattggcctcacactaccagggccaatgcccaaaataaggagttccaatttggggccaaatgaggaaggacacagactctgccctgggatctcctgtgctagcggccaatgacaaatccagtcattggccaccagccacctctgcagtggggaccacactagcagccctgactccacactcctcctggggacccaagaggcagtgttgctgtctgcntgtccaccttggaatctggctgaactggctggnaggaccaagactgcggctggggtgggcagggaagggaagccgggggctgctgtgagggatcttggagcttccctgtagcccaccttccccttgcttcatgtttgtagaggaaccttgtgccggcca

SEQ ID NO: 458 - DNA, 473 bp, human
accagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactc

SEQ ID NO: 459 - DNA, 379 bp, human
ttcttttctaaccaagaggctggttttggcatctctgtcccattccctgggatctggtggtcagccctaggataaaaagccagggctggagaacaagaaagggccaggagatggaattccttcaggccggcacccacaccctaggacatgtaagccctcatgtccaagggagcctcatgcagatagtaggaaatcaggtctggaaatttaaaaataaaaggcatgagactaaggctatctgcttcccttatgccctgactggagaggggagggaggagaggcaaggcccacagagggcatcccagctaggccttgggatggctgcagtgaggagaaatcccgggaactgtattgacacaaagattcttattgcacttgt

SEQ ID NO: 460 - DNA, 513 bp, human
  misc_feature (45)..(109): n is a, c, g, or t
gctcggctcggctccatgacaacgtccacgccgtcttccaggagnnnnnnnnnnnnnnnnnnnnnnnnnnnnnnnnnnnnnnnnnnnnnnnnnnnnnnnnnnnnnnnnncgtgnnnnnnnnnnnnnnncggggcgtcccagcgacgcgggaaggggcagggcgctgacctgctgctgagctggctgggctggacccggtccctaggctgtgaccgccgaactccactgcaacagacgggcgccaccaaagccagnccctgaggcctgggagtcctggactgagaaagggggttcctgggcccacctgctctgtgtagggctcgtcctgcggtgcccgagaatcactcgctaacccctatgcccggtcccggaccgacatcctggagccgcctgtgcagcctgatgccccctcgtggctgctcccagggctgcacctgccaggacctaatgttcttaggtccctctggccagaacccacacccggccccttcccacctgtcatactggtaactg

SEQ ID NO: 461 - DNA, 65 bp, human
ggaaagcatgtctgctgggtgtgaccatgtttcctctcaataaagttcccctgtgacactcaaaa

SEQ ID NO: 462 - DNA, 483 bp, human
  misc_feature (51)..(51): n is a, c, g, or t
ccaggaaactgcaggggctcacggaggaagtgctgcctgggccagggggancagctttcctccgtagagaccatgtgcagaacacttctgctgtgccaagaacatgagggagccagtgttttgtcagcaggaagaaagggcctgctgggatgaaagtgggaaggaaacagggttgngtagtcaggagacacctcaggggcaacagcacaggcccagagtacctgctgcctccactgcgtctgtcctggggtcatgaggatgctgaggttgacgacaggttccaggtcctttcactcctttggccaaaggttgggggtaggtggncccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgtgtactccgcagctgtttggggtgggacatttctgtacttctcgatt

SEQ ID NO: 463 - DNA, 457 bp, human
  misc_feature (264)..(264): n is a, c, g, or t
gttgtgccctctgagtgtctggctgatcacatcagagaggtctgcgtggcagtttggggctgtcacgtgaccagtgacccacactctctgctgcccagtactgccaagtggggagggtcctgcctttttctctgccccaggtctgggacgcaggtgatgccagccaggcccaggagtgcccagcatcccccaactgatgacacagtagcactgattctgtcttttcctcagaatctggcctttttccatggcaatgaggtgggncccagcctcctctaaagtgactttgtttctgcacagttgtaactgctcttggggatgtcagtgaggctgggagcagggagccacgggatgctgagagaggaggcccgagaggacaccccaccctccagcgtggcctttgatccagacttagggacgaggctgtcactggtgggcaccctctgttcctgtttgt

SEQ ID NO: 464 - DNA, 442 bp, human
  misc_feature (319)..(334): n is a, c, g, or t
agatcttggcccaaaccagcccaggcagtctaaagaaaactgggttggtgcttcttggaatgaaggcaaaaatctgaaattgttagtgtctcagtaatcctgatttagaacccaggctttttgtaacaatgtgttttcttgcctaaattcatttatctggcatcatcagagtactaacatgtttatatttcagatatccaaagcttaccactttaggtgatgaatctttactattttagcccttttttgatgagactatttgtctaaagtgaatcatttgttcttgccttattaaacagagtagatggaaaacaatttnnnnnnnnnnnnnnnnatttctttatgaatatgaataattgttctatgctttaataatctattgtgaggaaactactaagaaatatgttggtgtgtttgtccttacttgaaatgggtctgtatt

SEQ ID NO: 465 - DNA, 539 bp, human
  misc_feature (31)..(31): n is a, c, g, or t
aacatcgaggatgaaggcagctccttctatngggtctccagccagtatgaganccccgagaannnnnnnnnnnnnnnnnnnnnnnnnnnnnnnnnnnnnnnnnnnnnnnnnntggagaaagttgagacagagtatgctcgctatgagaatggacactactcttaccgcatccaccggtccccgctctgtgagtacatgatcaacttcatccacaagctcaagcacctccctgagaagtacatgatgaacagcgtgctggagaacttcaccatcctgcaggtggtcaccaacagagacacacaggagaccttgctgtgcattgcctatgtctttgaggtgtcagccagtgagcacggggctcagcaccacatctacaggctggtgaaagaatgagagactcggggagcagggaggggggaagagacgtgtgtgcaggaaacggggacgtggggaggggacctgcaggggcagccccctgaagtgccaagagagctgagaggagcagttgtgactctacccaggaacaaactgtgcctgaa

SEQ ID NO: 466 - DNA, 545 bp, human
  misc_feature (62)..(82): n is a, c, g, or t
caagctggactgcataaagattggtatggccttagctcttagccaaacaccttcctgacacnnnnnnnnnnnnnnnnnnnnngatcgtggtggtgttcctcnnnnnnnnnnnnnnnnnnnnnnnnnnnnnnnnnncgggagttcctgttaaaggtcaagacactgtcaaaggccgtgttccattcaatggacaagatcccgttaaaggacaagtttcagttaaaggtcnnnnnnnnnnnnnnnnncaagagccagtcaaaggtccagtctccactaagcctggctcctgccccattatcttgatccggtgcgccatgttgaatccccctaaccgctgcttgaaagatactgannnnnnnnnnnnnnngaagtgctgtgaaggctcttgcgggatggcctgtttcgttccccagtgagagggnnnnnnnnnnnnnnnnnnnnnnnnnnnnnnnnnnnnnnnnnnnnnnnnnnngtcctaagtccctgctgcccttccccttcccacactgtccattcttcctcccattcaggatgcccacggctgg

SEQ ID NO: 467 - DNA, 274 bp, human
  misc_feature (26)..(47): n is a, c, g, or t
cctgaacacattcctcagtgagatgnnnnnnnnnnnnnnnnnnnnnntataaccaagtgtccaccaactacctgctaagagccgggagcatggaacgtgttgggatttagagaacattatctgagaaaagagttcacttcctgctcccaggatatttctcttttctgtttatgaagtacaacccatgctgctaagatgcgagcaggaagaggcatcctttgctaaatcctgtttgaatgtcattgtaaataaagcctctgctctcagatgtaaa

SEQ ID NO: 468 - DNA, 238 bp, human
  misc_feature (74)..(74): n is a, c, g, or t
atcctggcaccacacgggtggcctacctcccggactgccctgagggcaacaaggtgctgaccctgttccgcaangcgtttgaccagcgtctcaccttcactatcggcacgtccatgaccacagggagaccgaatgtcatcacctggaacgacatccaccacaagaccagctgcacagggggancccagctgtttgggtacccagaccccacctacctgacccgggtgcaagaggagct

SEQ ID NO: 469 - DNA, 475 bp, human
  misc_feature (76)..(76): n is a, c, g, or t
ggtccagccggtccaagcacatggcctcccatctgggagagcccactgtcccactcccacatgtctgggcacctgncctgggctgaggccaggctgctccaggggcctcctgcgccctcacctgccacagagcaacccaggttaaatacagcccatgcacaaagccacaggnccaaagcctatggaattgtttttaatcatcaaatttaaccattttcnataactggtntcctggaggtgtgcagtgcccccttgcctcttcaaacctacagcttctctttgnccatttgtggatttcacatcactccacacagaaacnnnnnnnnnnnnnnnnnnnagtctttgccttcttccagctgcctcgacacagcactgtggcctgtccctattgcccaggcacgccatttccaagggcaggaaggggcagtgtcctgaagcccatcttttctgtgactgtcttaggtgatgtgtagcc

SEQ ID NO: 470 - DNA, 448 bp, human
  misc_feature (29)..(29): n is a, c, g, or t
cctctgagtcccagggacctgggcaggcncaagcccaccacgagcatcatccagtcctcagccctaatctgcncttaggagtccaggctgcaccctggagatcccanacctagccccctagtgggacaaggacctgaccctcctgcccgcatacacaacccatttcccctggtgagccacttggcagcatatgtaggtaccagctcaaccccacgcaagttcctgagctgaacatggagcaaggggagggtgacttctctccacatagggagggcttagagctcacagccttgggaagtgagactagaagaggggagcagaaagggaccttgagtagacaaaggccacacacatcattgtcattactgttttaattgtctggcttctctctggactgggagctcagtgaggattctgaccagtgacttacacaaaaggcgctctatac

SEQ ID NO: 471 - DNA, 420 bp, human
  misc_feature (102)..(102): n is a, c, g, or t
gagcttgtctctaagatatatacctcttccttttgtcttttgctgtaagctttgaccttttgcagatctgatgaaaatacaacctcttattgtatagtttgnccttgattataagccatnagtaaatcngagctgttncgncatttttgcaggccttgcattttctactgggaggttcatcaaaccttnccacttagcaatagnccctganctcaggcagaatgcctcataaattagccttcgaaagaaaagtgcacgctcagacagtgtgtggaggggcaggagcgtgatgcagacaaggacttagtcatgactgannnnnnnnnnnnnnnnnnnaatggcttatggggaacattgtgagcttcctacctgacatgttggcagaagtagaatattaggatcctcaagcatctctaccgt

(19) United States
(12) Patent Application Publication
(10) Pub. No.: US 2006/0252057 A1
(43) Pub. Date: Nov. 9, 2006

(54) Lung cancer prognostics

(76) Inventors: Mitch Raponi, San Diego, CA (US); Jack X. Yu, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/290,215

(22) Filed: Nov. 30, 2005

Related U.S. Application Data
(60) Provisional application No. 60/632,053, filed on Nov. 30, 2004.
(60) Provisional application No. 60/655,573, filed on Feb. 23, 2005.

(51) Int. Cl. C12Q 1/68; G01N 33/574

(52) U.S. Cl. 435/6; 435/7.23

(57) ABSTRACT

A method of providing a prognosis of lung cancer is conducted by analyzing the expression-of a group of genes. Gene expression profiles in a variety of medium such as microarrays are included as are kits that contain them.

### STATEMENT REGARDING FEDERALLY SPONSORED RESEARCH OR DEVELOPMENT

No government funds were used to make this invention.

### REFERENCE TO SEQUENCE LISTING, OR A COMPUTER PROGRAM LISTING COMPACT DISK APPENDIX

Reference to a “Sequence Listing,” a table, or a computer program listing appendix submitted on a compact disc and an incorporation by reference of the material on the compact disc including duplicates and the files on each compact disc shall be specified.

### BACKGROUND

This application claims the benefit of U.S. Patent Application No. 60/632,053, filed Nov. 30, 2005 which is incorporated herein by reference.

This invention relates to prognostics for lung cancer based on the gene expression profiles of biological samples.

Lung cancer is the leading cause of cancer deaths in developed countries killing about 1 million people worldwide each year. An estimated 171,900 new cases are expected in 2003 in the US, accounting for about 13% of all cancer diagnoses. Non-small cell lung cancer (NSCLC) represents the majority (˜75%) of bronchogenic carcinomas while the remainder is small cell lung carcinomas (SCLC). NSCLC is comprised of three main subtypes: 40% adenocarcinoma, 40% squamous, and 20% large cell cancer. Adenocarcinoma has replaced squamous cell carcinoma as the most frequent histological subtype over the last 25 years, peaking the early 1990's. This may be associated with the use of “low tar” cigarettes resulting in deeper inhalation of cigarette smoke. Wingo et al. (1999). The overall 10-year survival rate of patients with NSCLC is a dismal 8-10%.

Approximately 25-30% of patients with NSCLC have stage I disease and of these 35-50% will relapse within 5 years after surgical treatment. Depending upon stage, adenocarcinoma has a higher relapse rate than squamous cell carcinoma with approximately 65% and 55% of SCC and adenocarcinoma patients surviving at 5 years, respectively. Mountain et al. (1987). Currently, it is not possible to identify those patients with a high risk of relapse. The ability to identify high-risk patients among the stage I disease group will allow for the consideration of additional therapeutic intervention leading to the potential for improved survival. Indeed, recent clinical trials have shown that adjuvant therapy following resection of lung tumors can lead to improved survival. Kato et al. (2004). Specifically, Kato et al. demonstrated that adjuvant chemotherapy with uracil-tegafur improves survival among patients with completely resected pathological stage I adenocarcinoma, particularly T2 disease.

Microarray gene expression profiling has recently been utilized to define prognostic signatures in patients with lung adenocarcinomas, (Beer et al. (2002)) however, no large studies have investigated gene expression profiles of prognosis in the squamous cell carcinoma population. Here, we have profiled 134 SCC samples and 10 normal matched lung samples on the Affymetrix U133A chip. Hierarchical clustering and Cox modeling has identified genes that correlate with patient prognosis. These signatures can be used to identify patients who may benefit from adjuvant therapy following initial surgery.

### SUMMARY OF THE INVENTION

The present invention provides a method of assessing lung cancer status by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicative of lung cancer status.

The present invention provides a method of staging lung cancer patients by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicative of the lung cancer stage.

The present invention provides a method of determining lung cancer patient treatment protocol by obtaining a biological sample from a lung cancer patient; and measuring Biomarkers associated with Marker genes corresponding to those selected from Table 1, Table 4, Table 5 or Table 7 where the expression levels of the Marker genes above or below predetermined cut-off levels are sufficiently indicative of risk of recurrence to enable a physician to determine the degree and type of therapy recommended to prevent recurrence.

The present invention provides a method of treating a lung cancer patient by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicate a high risk of recurrence and; treating the patient with adjuvant therapy if they are a high risk patient.

The present invention provides a method of determining whether a lung cancer patient is high or low risk of mortality by obtaining a biological sample from a lung cancer patient; and measuring Biomarkers associated with Marker genes corresponding to those selected from Table 4 where the expression levels of the Marker genes above or below pre-determined cut-off levels are sufficiently indicative of risk of mortality to enable a physician to determine the degree and type of therapy recommended.

The present invention provides a method of generating a lung cancer prognostic patient report by determining the results of any one of the methods described herein and preparing a report displaying the results and patient reports generated thereby.

The present invention provides a composition comprising at least one probe set selected from the group consisting of: Marker genes corresponding to those selected from Table 1, Table 4, Table 5 or Table 7.

The present invention provides a kit for conducting an assay to determine lung cancer prognosis in a biological sample comprising: materials for detecting isolated nucleic acid sequences, their complements, or portions thereof of a combination of genes selected from the group consisting of Marker genes corresponding to those selected from Table 1, Table 4, Table 5 or Table 7.

The present invention provides articles for assessing lung cancer status comprising: materials for detecting isolated nucleic acid sequences, their complements, or portions thereof of a combination of genes selected from the group consisting of Marker genes corresponding to those selected from Table 1, Table 4, Table 5 or Table 7.

The present invention provides a microarray or gene chip for performing the method described herein.

The present invention provides a diagnostic/prognostic portfolio comprising isolated nucleic acid sequences, their complements, or portions thereof of a combination of genes selected from the group consisting of Marker genes corresponding to those selected from Table 1, Table 4, Table 5 or Table 7.

### BRIEF DESCRIPTION OF THE DRAWINGS

FIG. 1 depicts hierarchical clustering of 129 lung SCC patients.

FIG. 2 depicts plots of AUC vs. number of genes.

FIG. 3 depicts error rates of LOOCV v various cutoffs in the 65-sample training set.

FIG. 4 depicts Kaplan Meier plots of the 50-gene signature in the testing set.

FIG. 5 depicts unsupervised clustering identifies epidermnal differentiation pathway as being down-regulated in high-risk patients. A. Clustering of patients based on top 121 showed two clusters of patients. The majority of genes in cluster I were down-regulated (green). B. List of 20 genes associated with epidermal differentiation pathway. C. Kaplan Meier curve of clustered patient groups defined by the-20 epidermal-related genes.

FIG. 6 depicts verification of gene expression data using real-time RT-PCR. Four genes (NTRK2, FGFR2, VEGF, KRT13) were selected for RT-PCR. Expression correlate very well with Affymetrix chip data (R=0.71-0.96).

### DETAILED DESCRIPTION OF THE INVENTION

Non-small cell lung cancer (NSCLC) represents the majority (˜75%) of lung carcinomas and is comprised of three main subtypes: 40% squamous, 40% adenocarcinoma, and 20% large cell cancer. Approximately 25-30% of patients with NSCLC have stage I disease and of these 35-50% will relapse within 5 years after surgical treatment. Current histopathology and genetic biomarkers are insufficient for identifying patients who are at a high risk of relapse. As described in the present invention, 129 primary squamous cell lung carcinomas and 10 matched normal lung tissues were profiled using the Affymetrix U133A gene chip. Unsupervised hierarchical clustering identified two clusters of patients with lung carcinoma that had no correlation with stage of disease but had significantly different median overall survival (p=0.036). Cox proportional hazard models were then utilized to identify an optimal set of 50 genes (Table 1) in a 65 patient training set that significantly predicted survival in a 64 patient test set. This signature achieved 52% specificity and 82% sensitivity and provided an overall predictive value of 71%. Kaplan-Meier analysis showed clear significant stratification of high and low risk patients (p=0.0075). The identification of prognostic signatures allows identification of patients with high-risk squamous cell lung carcinoma who could benefit from adjuvant therapy following initial surgery.

TABLE 1

| SEQ ID NO: | Rank |
|---|---|
| 228 | 1 |
| 284 | 2 |
| 76 | 3 |
| 124 | 4 |
| 281 | 5 |
| 86 | 6 |
| 303 | 7 |
| 311 | 8 |
| 443 | 9 |
| 287 | 10 |
| 13 | 11 |
| 378 | 12 |
| 362 | 13 |
| 18 | 14 |
| 79 | 15 |
| 230 | 16 |
| 416 | 17 |
| 409 | 18 |
| 78 | 19 |
| 420 | 20 |
| 58 | 21 |
| 53 | 22 |
| 254 | 23 |
| 91 | 24 |
| 270 | 25 |
| 446 | 26 |
| 4 | 27 |
| 310 | 28 |
| 42 | 29 |
| 10 | 30 |
| 80 | 31 |
| 12 | 32 |
| 440 | 33 |
| 75 | 34 |
| 60 | 35 |
| 63 | 36 |
| 283 | 37 |
| 29 | 38 |
| 221 | 39 |
| 279 | 40 |
| 280 | 41 |
| 267 | 42 |
| 189 | 43 |
| 103 | 44 |
| 194 | 45 |
| 268 | 46 |
| 252 | 47 |
| 461 | 48 |
| 372 | 49 |
| 414 | 50 |

A Biomarker is any indicia of the level of expression of an indicated Marker gene. The indicia can be direct or indirect and measure over- or under-expression of the gene given the physiologic parameters and in comparison to an internal control, normal tissue or another carcinoma. Biomarkers include, without limitation, nucleic acids (both over and under-expression and direct and indirect). Using nucleic acids as Biomarkers can include any method known in the art including, without limitation, measuring DNA amplification, RNA, micro RNA, loss of heterozygosity (LOH), single nucleotide polymorphisms (SNPs, Brookes (1999)), microsatellite DNA, DNA hypo- or hyper-methylation. Using proteins as Biomarkers can include any method known in the art including, without limitation, measuring amount, activity, modifications such as glycosylation, phosphorylation, ADP-ribosylation, ubiquitination, etc., imunohistochemistry (IHC). Other Biomarkers include imaging, cell count and apoptosis markers.

The indicated genes provided herein are those associated with a particular tumor or tissue type. Marker gene may be associated with numerous cancer types but provided that the expression of the gene is sufficiently associated with one tumor or tissue type to be identified using the algorithm described herein to be specific for a lung cancer cell, the gene can be using in the claimed invention to determine cancer status and prognosis. Numerous genes associated with one or more cancers are known in the art. The present invention provides preferred Marker genes and even more preferred Marker gene combinations. These are described herein in detail.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with a tumor or tissue type. The preferred Marker genes are described in more detail in Table 8.

The present invention provides a method of assessing lung cancer status by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicative of lung cancer status.

The present invention provides a method of staging lung cancer patients by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicative of the lung cancer stage. The stage can correspond to any classification system, including, but not limited to the TNM system or to patients with similar gene expression profiles.

The present invention provides a method of determining lung cancer patient treatment protocol by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are sufficiently indicative of risk of recurrence to enable a physician to determine the degree and type of therapy recommended to prevent recurrence.

The present invention provides a method of treating a lung cancer patient by obtaining a biological sample from a lung cancer patient; and measuring Biomarkers associated with Marker genes corresponding to those selected from Table 1, Table 4, Table 5 or Table 7 where the expression levels of the Marker genes above or below pre-determined cut-off levels are indicate a high risk of recurrence and; treating the patient with adjuvant therapy if they are a high risk patient.

The present invention provides a method of determining whether a lung cancer patient is high or low risk of mortality by obtaining a biological sample from a lung cancer patient; and measuring Biomarkers associated with Marker genes corresponding to those selected from Table 4 where the expression levels of the Marker genes above or below pre-determined cut-off levels are sufficiently indicative of risk of mortality to enable a physician to determine the degree and type of therapy recommended.

In the above methods, the sample can be prepared by any method known in the art including, but not limited to, bulk tissue preparation and laser capture microdissection. The bulk tissue preparation can be obtained for instance from a biopsy or a surgical specimen.

In the above methods, the gene expression measuring can also include measuring the expression level of at least one gene constitutively expressed in the sample.

In the above methods, the specificity is preferably at least about 40% and the sensitivity at least at least about 80%.

In the above methods, the pre-determined cut-off levels are at least about 1.5-fold over- or under-expression in the sample relative to benign cells or normal tissue.

In the above methods, the pre-determined cut-off levels have at least a statistically significant p-value over-expression in the sample having metastatic cells relative to benign cells or normal tissue, preferably the p-value is less than 0.05.

In the above methods, gene expression can be measured by any method known in the art, including, without limitation on a microarray or gene chip, nucleic acid amplification conducted by polymerase chain reaction (PCR) such as reverse transcription polymerase chain reaction (RT-PCR), measuring or detecting a protein encoded by the gene such as by an antibody specific to the protein or by measuring a characteristic of the gene such as DNA amplification, methylation, mutation and allelic variation. The microarray can be for instance, a cDNA array or an oligonucleotide array. All these methods and can further contain one or more internal control reagents.

The present invention provides a method of generating a lung cancer prognostic patient report by determining the results of any one of the methods described herein and preparing a report displaying the results and patient reports generated thereby. The report can further contain an assessment of patient outcome and/or probability of risk relative to the patient population.

The present invention provides a composition comprising at least one probe set selected from the group consisting of: Marker genes corresponding to those selected from Table 1, Table 4, Table 5 or Table 7.

The present invention provides a kit for conducting an assay to determine lung cancer prognosis in a biological sample comprising: materials for detecting isolated nucleic acid sequences, their complements, or portions thereof of a combination of genes selected from the group consisting of Marker genes corresponding to those selected from Table 1, Table 4, Table 5 or Table 7. The kit can further comprise reagents for conducting a microarray analysis, and/or a medium through which said nucleic acid sequences, their complements, or portions thereof are assayed.

The present invention provides articles for assessing lung cancer status comprising: materials for detecting isolated nucleic acid sequences, their complements, or portions thereof of a combination of genes selected from the group consisting of Marker genes corresponding to those selected from Table 1, Table 4, Table 5 or Table 7. The articles can further contain reagents for conducting a microarray analysis and/or a medium through which said nucleic acid sequences, their complements, or portions thereof are assayed.

The present invention provides a microarray or gene chip for performing the method of claim 1, 2, 5, 6 or 7. The microarray can contain isolated nucleic acid sequences, their complements, or portions thereof of a combination of genes selected from the group consisting of Marker genes corresponding to those selected from Table 1, Table 4, Table 5 or Table 7. Preferably, the microarray is capable of measurement or characterization of at least 1.5-fold over- or under-expression. Preferably, the microarray provides a statistically significant p-value over- or under-expression. Preferably, the p-value is less than 0.05. The microarray can contain a cDNA array or an oligonucleotide array and/or one or more internal control reagents.

The present invention provides a diagnostic/prognostic portfolio comprising isolated nucleic acid sequences, their complements, or portions thereof of a combination of genes selected from the group consisting of Marker genes corresponding to those selected from Table 1, Table 4, Table 5 or Table 7. Preferably, the portfolio is capable of measurement or characterization of at least 1.5-fold over- or under-expression. Preferably, the portfolio provides a statistically significant p-value over- or under-expression. Preferably, the p-value is less than 0.05.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide diagnosis, status, prognosis and treatment protocol for lung cancer patients.

Sample preparation requires the collection of patient samples. Patient samples used in the inventive method are those that are suspected of containing diseased cells such as cells taken from a nodule in a fine needle aspirate (FNA) of tissue. Bulk tissue preparation obtained from a biopsy or a surgical specimen and Laser Capture Microdissection (LCM) are also suitable for use. LCM technology is one way to select the cells to be studied, minimizing variability caused by cell type heterogeneity. Consequently, moderate or small changes in Marker gene expression between normal or benign and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, a gene expression profile is obtained using a Biomarker, for genes in the appropriate portfolios.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in U.S. Patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

Gene expression profiles can also be displayed in a number of ways. The most common method is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including “GENESPRING” from Silicon Genetics, Inc. and “DISCOVERY” and “INFER” software from Partek, Inc.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated Markers used in the methods of the invention are described in the Examples. The genes that are differentially expressed are either up regulated or down regulated in patients with various lung cancer prognostics. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up- or down-regulated relative to the baseline level using the same measurement method.

Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic markers, it is often desirable to use the fewest number of markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in US patent publication number 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is one option. Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (“CA 27.29”)). A range of such markers exists including such analytes as CA 27.29. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The invention is further illustrated by the following non-limiting examples. All references cited herein are hereby incorporated herein.

### EXAMPLES

Genes analyzed according to this invention are typically related to full-length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### Example 1

Methods

Patient Population

134 fresh frozen, surgically resected lung SCC and 10 matched normal lung samples from 133 individual patients (LS-71 and LS-136 were duplicate samples from different areas of the same tumor) from all stages of squamous cell lung carcinoma were evaluated in this study. These samples were collected from patients from the University of Michigan Hospital between October 1991 and July 2002 with patient consent and Institutional Review Board (IRB) approval. Portions of the resected lung carcinomas were sectioned and evaluated by the study pathologist by routine hematoxylin and eosin (H&E) staining. Samples chosen for analysis contained greater than 70% tumor cells. Approximately one third of patients (with equal proportions for each stage) received radiotherapy or chemotherapy following surgery. Seventy-seven patients were lymph node negative. Follow-up data were available for all patients. The mean patient age was 68±10 (range 42-91) with approximately 45% of patients 70 years or older. One patient (LS-3) likely died of surgery-related causes and was therefore not utilized in identifying prognostic signatures. Also, three specimens had mixed histology and were also not included in prognostic profiling (LS-76, LS-84, LS-112).

Microarray Analysis

For isolation of RNA, 20 to 40 cryostat sections of 30 μm were cut from each sample, in total corresponding to approximately 100 mg of tissue. Before, in between, and after cutting the sections for RNA isolation, 5 μm sections were cut for hematoxylin and eosin staining to confirm the presence of tumor cells. Total RNA was isolated with RNAzol B (Campro Scientific, Veenendaal, Netherlands), and dissolved in DEPC (0.1%)-treated H₂O. About 2 ng of total RNA was resuspended in 10 μl of water and 2 rounds of the T7 RNA polymerase based amplification were performed to yield about 50 μg of amplified RNA. Quality of RNA was checked using the Agilent Bioanalyzer. The mean ribosomal ratio (28s/18s) for all samples was 1.5 (range: 1.0-2.1). Four micrograms of total RNA was amplified, labeled and aRNA was fragmented and hybridized to the Affymetrix U133A chip according to the manufacturer's instructions. Microarray data were extracted using the Affymetrix MAS 5 software. Global gene expression was scaled to an average intensity of 600 units. The data were then normalized using a spline quantile normalization method.

Statistical Analysis

Three complimentary statistical methods were performed to identify the optimal prognostic gene signature: Cox proportional-hazard regression modeling, bootstrapping, and a leave 20 percent out cross validation (L20OCV).

Univariate Cox proportional-hazard regression modeling was performed to identify genes that were significantly associated with overall survival. The Cox score was defined as the sum of the selected gene's log2-based chip signals multiplied by their z scores from the Cox regression. Similarly, Cox scores were calculated for patients in the testing set with the same selected genes from the training set. A series of cutoffs (percentile of risk index for the patients in the training set) was applied to predict the clinical outcome of patients in the testing set by comparing the patients° Cox score in the testing set with a cutoff for the risk index. If a patient's Cox score was higher than the cutoff, the patient was classified as “high risk”, otherwise, it is put in the “low risk” group. Kaplan-Meier analysis was performed to explore the survival characteristics of high-risk and low-risk patients. A cutoff of 3-year survival was employed since the majority of patients who will relapse in this population will have this occur within 3 years. Kiernan et al. (1993). Also many of these patients die due to non-cancer related illnesses after 3 years. Kiernan et al. (1993). This rationale was also employed when performing Cox modeling.

The bootstrap method was also employed to provide a more stringent means of defining prognostic genes. Using the same training and testing sets created above, 65 samples were selected, with replacement from the training set, and then Cox regression was performed on these samples. Each gene's P value and z score were recorded. This step was repeated 400 times thus giving 400 P values and z scores for each gene. For each gene, the top and bottom 5% of P values were removed and then the mean P value and the rank of each gene (based on the mean P value) were defined. Similarly, the top and bottom 5% z scores for each gene in the training set were removed and the sum of the remaining ones was calculated. Various numbers of top genes based on the mean P value were defined, their log2-based chip signal were multiplied with the sum of their z scores. This equated their Cox scores, namely, the risk index. The patients' Cox scores in the testing set was also calculated in this manner. Receiver operator characteristic (ROC) curves were drawn for patients in the training and testing sets and the area under the curve (AUC) values for each gene classifier was recorded. The AUC values were then plotted versus various numbers of gene classifiers to determine the optimal gene number that provides steady AUC values in the training set.

A L20OCV was also performed to confirm the optimal gene number of the classifier. First samples were partitioned into 5 groups with the same or very close numbers of samples. Five pairs of training and testing sets was generated with the training set consisting of 80% of samples and the testing set consisting of the remaining 20%. Therefore each sample was chosen exactly once in a testing set. Cox regression modeling was performed to select the top prognostic genes (from 2 to 200) in the training set and the selected genes were tested in the corresponding testing set. ROC was performed to calculate the AUC. The mean AUC of the 5 testing sets for gene number from 2 to 200 was calculated. This was repeated 100 times and the mean of 100 AUC's for gene numbers from 2 to 200 was then calculated. The mean AUC versus gene number (2 to 200) was plotted and the optimal number of genes in the signature was selected.

Hierarchical clustering was performed with GeneSpring7.0 (Silicon Genetics) to identify major clusters of patients and investigate their association with patient co-variates. Prior to clustering genes that had a coefficient of variation (CV) smaller than 0.3 (arbitrarily chosen) were removed so as to reduce the impact of genes that displayed minimal change in expression across the dataset. Thus a dataset with 11,101 genes was created for clustering analysis. The signal intensity of each gene was divided by the median expression level of that gene from all patients. Samples were clustered using Pearson correlation as measurement of similarity. Genes were clustered in the same way.

Results

Microarray Profiling

141 of the 144 microarrays gave excellent data (% present>40, scaling factor<10) while the remaining 3 samples (LS76, LS78, LS82) gave acceptable results (% present>30, scaling factor<15). Table 2 shows the clinical-pathological staging of the 134 SCC samples analyzed by microarray. All samples were included in initial clustering analysis. Genes were filtered from the dataset if they were not called present in at least 10% of all samples (including normal). This left 14,597 genes for analysis.

TABLE 2
Patient samples by stage

| Clinical | Number | Pathological | |
|---|---|---|---|
| Stage | (%) | Stage | Number |
| 1a | 28 (20) | T1 N0 M0 | 27 |
| 1b | 50 (35) | T2 N0 M0 | 48 |
| IIA | 7 (5) | T1 N1 M0 | 6 |
| IIB | 31 (22) | T1 N1 M0 | 30 |
| IIIA | 19 (14) | T2 N2 M0 | 10 |
| | | T3 N0 M0 | 1 |
| | | T3 N1 M0 | 3 |
| | | T3 N2 M0 | 4 |
| IIIB | 5 (4) | T4 N0 M0 | 1 |
| | | T4 N1 M0 | 3 |
| | | T4 N2 M0 | 1 |

Note.
One duplicate stage IIb, 77 lymph node negative samples

Unsupervised Hierarchical Clustering

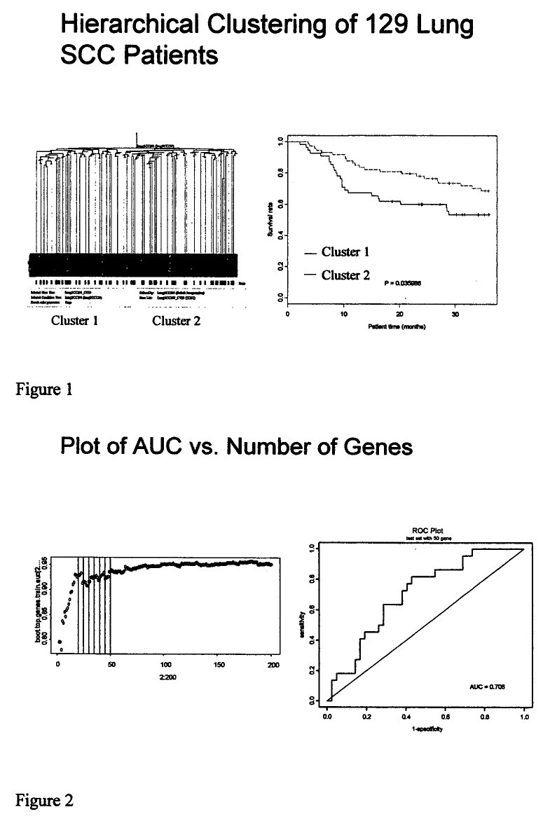

For unsupervised clustering the dataset was further filtered by removing genes (CV<30%) that had low variation of expression across the entire dataset. The 134 SCC and 10 normal lung samples were initially clustered based on unsupervised k-means clustering of the remaining 11,101 genes. The normal lung samples had a distinct profile from the carcinomas and clustered together. The 2 duplicate SCC samples (LS-71 and LS-136) clustered together demonstrating the reproducibility of the microarray analysis. Of the 133 unique patient carcinomas four were removed from further analysis since the patient either died due to surgery (LS3) or the sample had mixed histology (LS-76, LS-84, LS-112). When the 129 samples were clustered using the 11,101 genes two major clusters were formed, one with 55 patients and the other with 74 patients (FIG. 1A). No significant association between tumor stage, differentiation, or patient gender and the two clusters was identified. There were approximately equal proportions of each stage present in both clusters (cluster I consists of 31 stage I, 15 stage II and 9 stage III patients; cluster 2 consists of 42 stage I, 18 stage II and 14 stage III patients). However, the patients in cluster I and 2 showed significantly separated survival curves (FIG. 1B, p=0.036), indicating that expression profiles, irrespective of stage, existed that were associated with overall survival (FIG. 1B).

Identification of Prognostic Gene Signatures

To identify genes that could further stratify early stage patients into good and poor prognostic groups several complimentary statistical analyses were performed. This included: 1) Cox modeling on a training set and validating prognostic signatures on a test set of samples; 2) bootstrapping; and 3) L20OCV.

First, the 129 SCC samples were split into training and test sets with equal number of stages represented in both groups. Both groups showed similar overall median survival times. The 65-patient training set was analyzed using a bootstrapping method (see Methods section) to determine the optimal number of genes to be used in the prognostic signature. When increasing numbers of genes was plotted versus the AUC from a receiver operator characteristic analysis it could be seen that the signature performance began to plateau at around 50 genes (FIG. 2A). A L20OCV procedure was used to confirm the optimal number of prognostic genes in the 65-patient training set. The result showed that a signature has a stable performance when the number of genes reaches 50. Therefore, the top ranked 50 genes would be used as the signature. The 50-gene classifier demonstrated overall predictive value of 70% when used in the 64-patient test set (FIG. 2B).

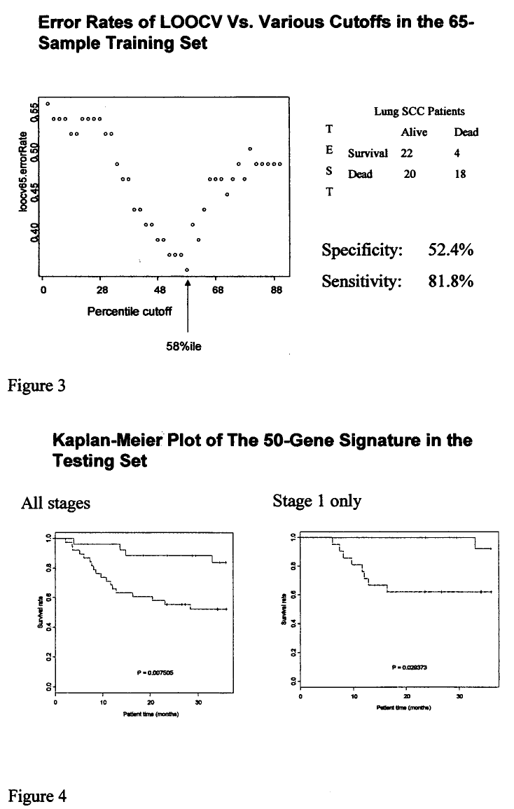

A LOOCV procedure was then used in the 65-patient training set to determine the optimal cutoff of the risk index. The error rates were calculated with various cutoffs. This indicated that cutoff at 58%ile gave the lowest error rate (FIG. 3). Therefore, the 58% ile of patients was used as the cutoff for determining survival. The performance of the prognostic signature was then examined in the testing set using this cutoff. The signature achieved 52.4% specificity and 81.8% sensitivity in the testing set (FIG. 3). Kaplan-Meier plot also showed good separation between predicted high-risk group of patients and low risk group of patients (p=0.0075). Multivariate analysis including sex, differentiation, stage, tumor size, age, and lymph node status was performed. None of the parameters except for the 50-gene signature had a significant p-value (Table 3). Kaplan-Meier analysis was also performed using the 50-gene signature and a risk cutoff of 58%. The high-risk group was well separated from the low risk group in all patients (p=0.0075, FIG. 4A) and when only those with stage 1 disease were tested (p 0.029; FIG. 4B).

TABLE 3
Multivariate Analysis

| Co-variate | P-value |
|---|---|
| 50 gene signature | 0.01 |
| Sex | 0.24 |
| Differentiation | 0.66 |
| Stage | 0.41 |
| T | 0.91 |
| Age | 0.35 |
| N | 0.99 |

### Example 2

Identification of a Robust Prognostic Signature

Although we used a bootstrap method to avoid random sampling issues in the training-testing method, a more robust prognostic signature might be identified if we use all 129 samples in the training set. Therefore, a gene signature was also selected by bootstrapping the entire 129-patient dataset. Genes were ranked based on their mean P value and the top 100 genes were identified (Table 4). Twenty-three of these genes were in common with the top 50 genes identified from the training-test method.

We had data on time to relapse (TTR) for 16 patients. The mean TTR was 21.7 months with 88% of patients relapsing within 3 years. Since the majority of patients who die after 3 years die from non-cancer related causes we chose a cutoff of 36 months for classifying patients who will have a lung cancer-related death. Our defined classifiers were tested with or without a 36-month cutoff. The signatures had a better performance in the testing set when a 3-year cutoff was employed. Therefore, a gene signature selected with the time limit is better than without the time limit.

TABLE 4

| SEQ ID NO: | Rank |
|---|---|
| 452 | 1 |
| 191 | 2 |
| 303 | 3 |
| 378 | 4 |
| 270 | 5 |
| 79 | 6 |
| 409 | 7 |
| 76 | 8 |
| 450 | 9 |
| 413 | 10 |
| 365 | 11 |
| 135 | 12 |
| 18 | 13 |
| 460 | 14 |
| 393 | 15 |
| 375 | 16 |
| 396 | 17 |
| 86 | 18 |
| 190 | 19 |
| 204 | 20 |
| 65 | 21 |
| 433 | 22 |
| 439 | 23 |
| 471 | 24 |
| 124 | 25 |
| 107 | 26 |
| 77 | 27 |
| 13 | 28 |
| 461 | 29 |
| 91 | 30 |
| 225 | 31 |
| 290 | 32 |
| 252 | 33 |
| 194 | 34 |
| 21 | 35 |
| 206 | 36 |
| 161 | 37 |
| 36 | 38 |
| 207 | 39 |
| 37 | 40 |
| 315 | 41 |
| 87 | 42 |
| 288 | 43 |
| 369 | 44 |
| 235 | 45 |
| 337 | 46 |
| 383 | 47 |
| 228 | 48 |
| 248 | 49 |
| 423 | 50 |
| 200 | 51 |
| 234 | 52 |
| 58 | 53 |
| 386 | 54 |
| 120 | 55 |
| 305 | 56 |
| 302 | 57 |
| 16 | 58 |
| 432 | 59 |
| 381 | 60 |
| 269 | 61 |
| 75 | 62 |
| 209 | 63 |
| 293 | 64 |
| 20 | 65 |
| 83 | 66 |
| 408 | 67 |
| 388 | 68 |
| 443 | 69 |
| 372 | 70 |
| 286 | 71 |
| 289 | 72 |
| 57 | 73 |
| 215 | 74 |
| 144 | 75 |
| 89 | 76 |
| 158 | 77 |
| 149 | 78 |
| 98 | 79 |
| 29 | 80 |
| 35 | 81 |
| 311 | 82 |
| 310 | 83 |
| 279 | 84 |
| 384 | 85 |
| 298 | 86 |
| 48 | 87 |
| 222 | 88 |
| 425 | 89 |
| 56 | 90 |
| 398 | 91 |
| 453 | 92 |
| 470 | 93 |
| 261 | 94 |
| 462 | 95 |
| 162 | 96 |
| 131 | 97 |
| 284 | 98 |
| 326 | 99 |
| 114 | 100 |

### Example 3

Identification of a High-Risk Sub-Group of SCC Patients

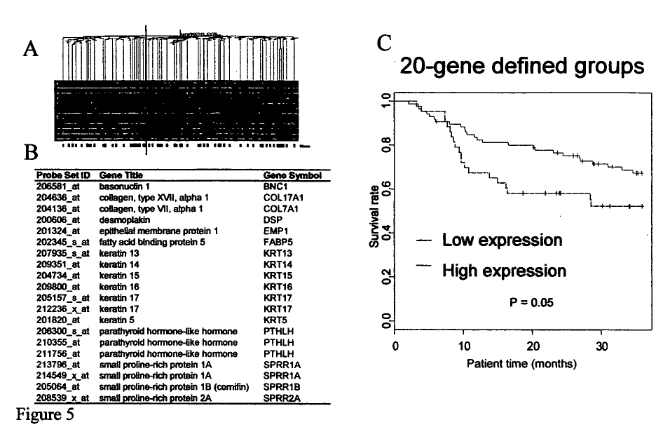

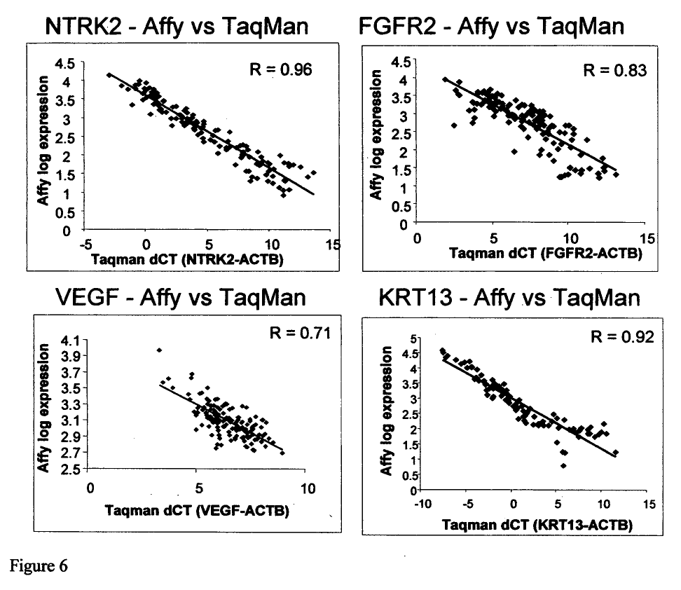

The unsupervised hierarchical clustering described above identified two main groups of patients that differed significantly in their overall survival. A bootstrap analysis performed on the two patient groups found 121 genes (non-unique) whose expression levels were significantly different between the high- and low-risk groups (p <0.001, mean difference>3-fold; Table 5). Interestingly, the majority of these genes (118) were down-regulated in the high risk group (FIG. 5A, cluster 1). Pathway analysis demonstrated that genes involved in epidermal development functions, including keratins and small-proline rich proteins, were significantly enriched for in this dataset. These data, shown in Table 6, indicate that there are two major subtypes of SCC one of which has a gene expression profile consistent with poor differentiation and as such tends to be more aggressive. When the genes only involved in epidermal differentiation (FIG. 5B) were used to cluster the patient samples the two prognostically differentiated groups were maintained (FIG. 5C). These data indicate that there are two major subtypes of SCC one of which has a gene expression profile consistent with poor differentiation and as such tends to be more aggressive. The lack of expression of epidermal differentiation genes may be associated with a subgroup of tumors that are de-differentiated and therefore more aggressive.

TABLE 5
121 genes significantly different between low- and high-risk clusters

| | Dunn-Sidak p- |
|---|---|
| SEQ ID NO: | value |
| 47 | 4.069E−08 |
| 52 | 0.001779787 |
| 61 | 4.78438E−06 |
| 64 | 3.94295E−08 |
| 70 | 6.14897E−11 |
| 71 | 5.40462E−10 |
| 72 | 4.99526E−07 |
| 91 | 1.17801E−09 |
| 92 | 0 |
| 93 | 1.51307E−07 |
| 94 | 0.00024053 |
| 97 | 3.25762E−06 |
| 101 | 0.000715044 |
| 102 | 4.042E−05 |
| 105 | 1.28648E−05 |
| 111 | 4.10746E−07 |
| 112 | 0.000129644 |
| 115 | 7.6587E−08 |
| 118 | 4.67009E−05 |
| 121 | 7.48718E−09 |
| 123 | 1.61815E−11 |
| 125 | 4.82759E−08 |
| 126 | 1.80901E−05 |
| 128 | 1.45634E−11 |
| 132 | 0.000571137 |
| 134 | 3.42792E−07 |
| 138 | 2.83176E−10 |
| 140 | 4.93018E−08 |
| 141 | 9.06164E−11 |
| 142 | 1.73482E−08 |
| 145 | 0 |
| 146 | 8.6277E−05 |
| 148 | 1.68459E−07 |
| 156 | 8.93603E−05 |
| 159 | 0 |
| 160 | 7.24383E−06 |
| 166 | 4.46788E−05 |
| 167 | 1.61815E−12 |
| 168 | 3.2363E−12 |
| 170 | 5.27808E−08 |
| 171 | 0 |
| 172 | 0 |
| 173 | 0 |
| 174 | 0 |
| 175 | 3.70691E−07 |
| 177 | 0.000964585 |
| 179 | 0.00023307 |
| 181 | 2.10853E−07 |
| 184 | 0.000261 |
| 185 | 1.22494E−09 |
| 186 | 0 |
| 188 | 8.3147E−08 |
| 192 | 0 |
| 193 | 1.33552E−06 |
| 194 | 0 |
| 195 | 8.04368E−07 |
| 196 | 0 |
| 198 | 1.78886E−07 |
| 213 | 0 |
| 214 | 0 |
| 216 | 1.77997E−11 |
| 219 | 1.44447E−07 |
| 223 | 6.79057E−08 |
| 229 | 2.21201E−09 |
| 231 | 0.000127662 |
| 232 | 0.000670091 |
| 233 | 0.000334014 |
| 236 | 0.000371339 |
| 237 | 5.35608E−10 |
| 238 | 0 |
| 243 | 0 |
| 245 | 1.5392E−07 |
| 246 | 3.77172E−06 |
| 251 | 9.51746E−06 |
| 253 | 1.61815E−12 |
| 257 | 7.19348E−07 |
| 259 | 3.2363E−12 |
| 260 | 0 |
| 262 | 0 |
| 263 | 1.61815E−12 |
| 278 | 3.2363E−12 |
| 285 | 3.95638E−09 |
| 313 | 3.06803E−07 |
| 318 | 0 |
| 320 | 1.10983E−05 |
| 321 | 2.86717E−06 |
| 322 | 0 |
| 323 | 1.46054E−05 |
| 324 | 2.65922E−05 |
| 331 | 0 |
| 332 | 1.77997E−10 |
| 333 | 0 |
| 341 | 3.60669E−08 |
| 348 | 0.001219264 |
| 349 | 4.42435E−08 |
| 353 | 0 |
| 357 | 9.21286E−05 |
| 358 | 2.91267E−09 |
| 360 | 1.67317E−09 |
| 366 | 0 |
| 367 | 1.06791E−07 |
| 371 | 0 |
| 373 | 0.000736609 |
| 397 | 1.53724E−10 |
| 402 | 0.001640004 |
| 405 | 1.89887E−05 |
| 407 | 0 |
| 418 | 7.28168E−11 |
| 419 | 1.13076E−08 |
| 424 | 2.83902E−05 |
| 426 | 0.001696015 |
| 429 | 2.33385E−05 |
| 435 | 2.53251E−06 |
| 445 | 8.59804E−08 |
| 457 | 0 |
| 458 | 0 |
| 459 | 0 |
| 463 | 9.60372E−09 |
| 468 | 4.52017E−06 |

TABLE 6
List of significantly enriched pathways

| | | | | GO. | |
|---|---|---|---|---|---|
| | Gene. | | Gene.#.On | Cate- | |
| GO.ID | Count | GO.Class | .U133a | gory | p.value |
| 8544 | 17 | epidermal | 56 | P | 7.31E−12 |
| | | differentiation | | | |
| 6325 | 3 | chromatin architecture | 12 | P | 2.75E−04 |
| 7586 | 3 | digestion | 15 | P | 7.08E−04 |
| 7156 | 4 | homophilic cell | 39 | P | 0.004886 |
| | | adhesion | | | |
| 7148 | 3 | cell shape and cell | 28 | P | 0.007914 |
| | | size control | | | |
| 7565 | 3 | pregnancy | 28 | P | 0.007914 |
| 165 | 2 | MAPKKKcascade | 15 | P | 0.008242 |
| 6805 | 2 | xenobiotic metabolism | 15 | P | 0.008242 |
| 7169 | 3 | receptor tyrosine | 41 | P | 0.029293 |
| | | kinase signaling | | | |
| 6832 | 2 | small molecule | 29 | P | 0.049333 |
| | | transport | | | |

### Example 4

Gene Expression Signatures for Prognosis of Lung Cancer.

Methods

Real-Time Quantitative RT-PCR

Total RNA samples were normalized by OD₂₆₀. Quality testing included analysis by capillary electrophoresis using a Bioanalyzer (Agilent). For aRNA, the Ribobeast™ 1-Round Aminoallyl-aRNA amplification kit (Epicentre) was used. All first-strand cDNA synthesis, second-strand cDNA synthesis, in vitro transcription of aRNA, DNase treatment, purification and other steps were performed according to the manufacturer's protocol. For each sample aRNA was reverse transcribed into first-stand cDNA and used for real-time quantitative RT-PCR. The first-strand cDNA synthesis reaction contained, 100 ng of aRNA, 1 μl of 50 ng/μl T7-Oligo(dT) primer, 0.25 μl of 10 mM dNTPs, 1 μl of 5× Superscript™ III Reverse Transcriptase Buffer, 0.25 μl of 200 U/μl Superscript™ III Reverse Transcriptase (Invitrogen Corp), 0.25 μl of 100 mM DTT and 0.25 μl of 0.3 U/μl RNase Inhibitor (Epicentre) in a total reaction volume of 5 μl.

Teal-time quantitative RT-PCR analyses were performed on the ABI Prism 7900HT sequence detection system (Applied Biosystems). Each reaction contained 10 μl of 2× TaqMan® Universal PCR Master Mix (Applied Biosystems), 5 μl of cDNA template, and 1 μl of 20× Assays-on-Demand Gene Expression Assay Mix (Applied Biosystems) in a total reaction volume of 20 μl. The PCR consisted of an UNG activation step at 50° C. for 2 min and initial enzyme activation step at 95° C. for 10 min, followed by 40 cycles of 95° C. for 15 sec, 60° C. for 1 min.

Immunohistochemistry

Immunohistochemistry (IHC) was performed on tissue microarrays containing 60 lung squamous cell carcinomas. Areas of the tumor that best represented the overall morphology were selected for generating a tissue microarray (TMA) block as previously described by Kononen et al. (1998). All controls stained negative for background.

Pathway Analysis

Pathway analysis was performed by first mapping the genes on the Affy U133A chip to the Biological Process categories of Gene Ontology (GO). The categories that had at least 10 genes on the U133A chip were used for subsequent pathway analyses. Genes that were selected from data analysis were mapped to the GO Biological Process categories. Then the hypergeometric distribution probability of the genes was calculated for each category. A category that had a p-value less than 0.05 and had at least two genes was considered over-represented in the selected gene list.

Identification of Core Set of Prognostic Genes

Briefly, 400 random training sets of 65 patients were selected from the 129 lung SCC patients. For each training set, Cox regression was performed to identify significant genes at the 5% significance level (i.e. P<0.05). 331 genes that are significant in more than 40% of the training sets are used as the core gene sets. These 331 genes are shown in Table 7.

Microarray Results Verification

To confirm the microarray results we initially performed TaqMan® quantitative RT-PCR on4 genes (FGFR2, KRT13, NTRK2, and VEGF). The correlation between the platforms ranged from 0.71 to 0.96 indicating the expression data were reproducible.

Immunohistochemistry was then performed on tissue microarrays to confirm expression of several of these proteins within the tumor cells. Various levels of expression of several keratins in addition to the tyrosine kinase proteins FGFR2 and NTKR2 in SCC cells was demonstrated.

Identification of a Core Set of Prognostic Genes

In the previous analysis a set of 50 genes was identified from a single training set of 65 patients. One problem with this approach is that the genes identified as predictors of prognosis can be unstable since the molecular signature strongly depends on the selection of patients in the training sets. The use of validation by repeated random sampling can avoid this instability. We therefore generated 400 random training sets of 65 patients from the 129 lung SCC patients and performed Cox regression to identify significant genes at the 5% significance level (i.e. P<0.05). 331 genes that were significant in more than 40% of the training sets were identified as a core set of prognostic genes in squamous cell lung cancer. These genes are SEQ ID NOs: in Table 7.

TABLE 7
331 Core genes

| | | | | | | | | |
|---|---|---|---|---|---|---|---|---|
| 1 | 2 | 3 | 5 | 6 | 7 | 8 | 9 | 11 |
| 13 | 14 | 15 | 16 | 17 | 18 | 20 | 21 | 22 |
| 23 | 24 | 25 | 26 | 27 | 28 | 29 | 30 | 31 |
| 32 | 33 | 34 | 35 | 36 | 37 | 38 | 39 | 40 |
| 41 | 42 | 43 | 44 | 45 | 46 | 48 | 49 | 50 |
| 51 | 54 | 55 | 56 | 57 | 58 | 59 | 62 | 65 |
| 66 | 67 | 68 | 69 | 73 | 74 | 75 | 76 | 77 |
| 79 | 80 | 81 | 82 | 83 | 84 | 85 | 86 | 87 |
| 88 | 89 | 90 | 91 | 92 | 95 | 96 | 98 | 99 |
| 100 | 104 | 106 | 107 | 108 | 109 | 110 | 113 | 114 |
| 116 | 117 | 119 | 120 | 122 | 124 | 127 | 129 | 130 |
| 133 | 134 | 135 | 136 | 137 | 139 | 141 | 143 | 147 |
| 149 | 150 | 151 | 152 | 153 | 154 | 155 | 157 | 159 |
| 161 | 163 | 164 | 165 | 166 | 169 | 176 | 178 | 180 |
| 182 | 183 | 187 | 190 | 191 | 194 | 197 | 199 | 200 |
| 201 | 202 | 203 | 204 | 205 | 206 | 207 | 208 | 209 |
| 210 | 211 | 212 | 215 | 217 | 218 | 220 | 222 | 224 |
| 225 | 226 | 227 | 228 | 234 | 235 | 239 | 240 | 241 |
| 242 | 244 | 247 | 248 | 249 | 250 | 252 | 254 | 255 |
| 256 | 258 | 261 | 263 | 264 | 265 | 266 | 269 | 270 |
| 271 | 272 | 274 | 275 | 276 | 282 | 283 | 284 | 286 |
| 288 | 289 | 290 | 291 | 292 | 293 | 294 | 295 | 296 |
| 297 | 298 | 299 | 300 | 301 | 302 | 303 | 304 | 305 |
| 306 | 307 | 308 | 309 | 310 | 311 | 312 | 314 | 315 |
| 316 | 317 | 319 | 325 | 327 | 328 | 329 | 330 | 334 |
| 335 | 336 | 337 | 338 | 339 | 340 | 342 | 343 | 344 |
| 345 | 346 | 347 | 350 | 351 | 352 | 354 | 355 | 356 |
| 359 | 361 | 363 | 364 | 365 | 368 | 369 | 370 | 372 |
| 374 | 375 | 376 | 377 | 378 | 379 | 380 | 381 | 382 |
| 383 | 384 | 385 | 386 | 387 | 388 | 389 | 390 | 391 |
| 392 | 393 | 394 | 395 | 396 | 398 | 399 | 400 | 401 |
| 403 | 404 | 406 | 409 | 410 | 411 | 412 | 413 | 415 |
| 417 | 420 | 421 | 422 | 423 | 425 | 427 | 428 | 430 |
| 431 | 432 | 433 | 434 | 436 | 437 | 438 | 439 | 441 |
| 442 | 443 | 444 | 447 | 448 | 449 | 450 | 451 | 452 |
| 453 | 454 | 455 | 456 | 460 | 461 | 462 | 464 | 465 |
| 466 | 467 | 469 | 470 | 471 | 472 | 473 | | |

Although the foregoing invention has been described in some detail by way of illustration and example for purposes of clarity of understanding, the descriptions and examples should not be construed as limiting the scope of the invention.

TABLE 8
SEQ ID NOs: and gene descriptions

| | | | | |
|---|---|---|---|---|
| 1 | 1255_g_at | guanylate cyclase activator 1A (retina) | GUCA1A | L36861 |
| 2 | 200619_at | splicing factor 3b, subunit 2 | SF3B2 | NM_006842 |
| 3 | 200650_s_at | lactate dehydrogenase A | LDHA | NM_005566 |
| 4 | 200727_s_at | ARP2 actin-related protein 2 homolog | ACTR2 | AA699583 |
| 5 | 200728_at | ARP2 actin-related protein 2 homolog | ACTR2 | BE566290 |
| 6 | 200737_at | phosphoglycerate kinase 1 | PGK1 | NM_000291 |
| 7 | 200795_at | SPARC-like 1 (mast9, hevin) | SPARCL1 | NM_004684 |
| 8 | 200810_s_at | cold inducible RNA binding protein | CIRBP | NM_001280 |
| 9 | 200811_at | cold inducible RNA binding protein | CIRBP | NM_001280 |
| 10 | 200824_at | glutathione S-transferase pi | GSTP1 | NM_000852 |
| 11 | 200836_s_at | microtubule-associated protein 4 | MAP4 | NM_002375 |
| 12 | 200840_at | lysyl-tRNA synthetase | KARS | NM_005548 |
| 13 | 200863_s_at | RAB11A, member RAS oncogene family | RAB11A | AI215102 |
| 14 | 200893_at | splicing factor, arginine/serine-rich 10 | SFRS10 | NM_004593 |
| 15 | 200951_s_at | cyclin D2 | CCND2 | AW026491 |
| 16 | 200970_s_at | stress-associated endoplasmic reticulum protein 1 | SERP1 | AL136807 |
| 17 | 200993_at | importin 7 | IPO7 | AA939270 |
| 18 | 201003_x_at | ubiquitin-conjugating enzyme E2 variant 1 | UBE2V1 | NM_003349 |
| 19 | 201033_x_at | ribosomal protein, large, P0 | RPLP0 | NM_001002 |
| 20 | 201047_x_at | RAB6A, member RAS oncogene family | RAB6A | BC003617 |
| 21 | 201067_at | proteasome (prosome, macropain) 26S subunit, | PSMC2 | BF215487 |
| | | ATPase, 2 | | |
| 22 | 201125_s_at | integrin, beta 5 | ITGB5 | NM_002213 |
| 23 | 201151_s_at | muscleblind-like | MBNL1 | BF512200 |
| 24 | 201152_s_at | muscleblind-like | MBNL1 | N31913 |
| 25 | 201154_x_at | ribosomal protein L4 | RPL4 | NM_000968 |
| 26 | 201170_s_at | basic helix-loop-helix domain containing, class B, 2 | BHLHB2 | NM_003670 |
| 27 | 201175_at | thioredoxin-related transmembrane protein 2 | TMX2 | NM_015959 |
| 28 | 201236_s_at | BTG family, member 2 | BTG2 | NM_006763 |
| 29 | 201251_at | pyruvate kinase, muscle | PKM2 | NM_002654 |
| 30 | 201286_at | syndecan 1 | SDC1 | Z48199 |
| 31 | 201287_s_at | syndecan 1 | SDC1 | NM_002997 |
| 32 | 201351_s_at | YME1-like 1 | YME1L1 | AF070656 |
| 33 | 201353_s_at | bromodomain adjacent to zinc finger domain, 2A | BAZ2A | AI653126 |
| 34 | 201361_at | hypothetical protein MGC5508 | MGC5508 | NM_024092 |
| 35 | 201447_at | TIA1 cytotoxic granule-associated RNA binding | TIA1 | H96549 |
| 36 | 201448_at | TIA1 cytotoxic granule-associated RNA binding | TIA1 | AL046419 |
| | | transcript variant 1 | | |
| 37 | 201449_at | TIA1 cytotoxic granule-associated RNA binding | TIA1 | AL567227 |
| | | transcript variant 1 | | |
| 38 | 201545_s_at | poly(A) binding protein, nuclear 1 | PABPN1 | NM_004643 |
| 39 | 201623_s_at | aspartyl-tRNA synthetase | DARS | BC000629 |
| 40 | 201667_at | gap junction protein, alpha 1 | GJA1 | NM_000165 |
| 41 | 201683_x_at | chromosome 14 open reading frame 92 | C14orf92 | BE783632 |
| 42 | 201718_s_at | erythrocyte membrane protein band 4.1-like 2 | EPB41L2 | BF511685 |
| 43 | 201725_at | chromosome 10 open reading frame 7 | C10orf7 | NM_006023 |
| 44 | 201779_s_at | ring finger protein 13 | RNF13 | AF070558 |
| 45 | 201780_s_at | ring finger protein 13 | RNF13 | NM_007282 |
| 46 | 201801_s_at | solute carrier family 29 (nucleoside transporters), | SLC29A1 | AF079117 |
| | | mem 1 | | |
| 47 | 201820_at | keratin 5 | KRT5 | NM_000424 |
| 48 | 201892_s_at | IMP (inosine monophosphate) dehydrogenase 2 | IMPDH2 | NM_000884 |
| 49 | 202006_at | protein tyrosine phosphatase, non-receptor type 12 | PTPN12 | NM_002835 |
| 50 | 202170_s_at | aminoadipate-semialdehyde dehydrogenase- | AASDHPPT | AF151057 |
| | | phosphopantetheinyl transferase | | |
| 51 | 202181_at | KIAA0247 | KIAA0247 | NM_014734 |
| 52 | 202219_at | solute carrier family 6, member 8 | SLC6A8 | NM_005629 |
| 53 | 202223_at | integral membrane protein 1 | ITM1 | NM_002219 |
| 54 | 202253_s_at | dynamin 2 | DNM2 | NM_004945 |
| 55 | 202288_at | FK506 binding protein 12-rapamycin assoc. pro 1 | FRAP1 | U88966 |
| 56 | 202349_at | torsin family 1, member A (torsin A) | TOR1A | NM_000113 |
| 57 | 202364_at | MAX interactor 1 | MXI1 | NM_005962 |
| 58 | 202397_at | nuclear transport factor 2 | NUTF2 | NM_005796 |
| 59 | 202418_at | Yip1 interacting factor homolog | YIF1 | NM_020470 |
| 60 | 202471_s_at | isocitrate dehydrogenase 3 (NAD+) gamma | IDH3G | NM_004135 |
| 61 | 202489_s_at | FXYD domain-containing ion transport regulator 3 | FXYD3 | BC005238 |
| 62 | 202496_at | autoantigen | RCD-8 | NM_014329 |
| 63 | 202503_s_at | KIAA0101 gene product | KIAA0101 | NM_014736 |
| 64 | 202504_at | ataxia-telangiectasia group D-associated protein | TRIM29 | NM_012101 |
| 65 | 202530_at | mitogen-activated protein kinase 14 | MAPK14 | NM_001315 |
| 66 | 202602_s_at | HIV TAT specific factor 1 | HTATSF1 | NM_014500 |
| 67 | 202746_at | integral membrane protein 2A | ITM2A | AL021786 |
| 68 | 202747_s_at | integral membrane protein 2A | ITM2A | NM_004867 |
| 69 | 202753_at | proteasome regulatory particle subunit p44S10 | P44S10 | NM_014814 |
| 70 | 202755_s_at | glypican 1 | GPC1 | AI354864 |
| 71 | 202756_s_at | glypican 1 | GPC1 | NM_002081 |
| 72 | 202831_at | glutathione peroxidase 2 | GPX2 | NM_002083 |
| 73 | 202887_s_at | DNA-damage-inducible transcript 4 | DDIT4 | NM_019058 |
| 74 | 202935_s_at | SRY-box 9 | SOX9 | AI382146 |
| 75 | 202990_at | phosphorylase, glycogen; liver | PYGL | NM_002863 |
| 76 | 203040_s_at | hydroxymethylbilane synthase | HMBS | NM_000190 |
| 77 | 203082_at | BMS1-like, ribosome assembly protein (yeast) | BMS1L | NM_014753 |
| 78 | 203190_at | NADH dehydrogenase (ubiquinone) Fe—S protein 8 | NDUFS8 | NM_002496 |
| 79 | 203196_at | ATP-binding cassette, sub-fam C (CFTR/MRP), | ABCC4 | AI948503 |
| | | mem 4 | | |
| 80 | 203211_s_at | myotubularin related protein 2 | MTMR2 | AK027038 |
| 81 | 203368_at | cysteine-rich with EGF-like domains 1 | CRELD1 | NM_015513 |
| 82 | 203372_s_at | suppressor of cytokine signaling 2 | SOCS2 | AB004903 |
| 83 | 203378_at | pre-mRNA cleavage complex II protein Pcf11 | PCF11 | AB020631 |
| 84 | 203491_s_at | translokin | PIG8 | AI123527 |
| 85 | 203494_s_at | translokin | PIG8 | NM_014679 |
| 86 | 203545_at | asparagine-linked glycosylation 8 homolog | ALG8 | NM_024079 |
| 87 | 203555_at | protein tyrosine phosphatase, non-receptor type 18 | PTPN18 | NM_014369 |
| 88 | 203573_s_at | Rab geranylgeranyltransferase, alpha subunit | RABGGTA | NM_004581 |
| 89 | 203589_s_at | transcription factor Dp-2 | TFDP2 | NM_006286 |
| 90 | 203611_at | telomeric repeat binding factor 2 | TERF2 | NM_005652 |
| 91 | 203638_s_at | fibroblast growth factor receptor 2 | FGFR2 | NM_022969 |
| 92 | 203639_s_at | fibroblast growth factor receptor 2 | FGFR2 | M80634 |
| 93 | 203691_at | protease inhibitor 3, skin-derived | PI3 | NM_002638 |
| 94 | 203726_s_at | laminin, alpha 3 | LAMA3 | NM_000227 |
| 95 | 203759_at | ST3 beta-galactoside alpha-2,3-sialyltransferase 4 | ST3GAL4 | NM_006278 |
| 96 | 203787_at | single-stranded DNA binding protein 2 | SSBP2 | NM_012446 |
| 97 | 203798_s_at | visinin-like 1 | VSNL1 | NM_003385 |
| 98 | 203809_s_at | v-akt murine thymoma viral oncogene homolog 2 | AKT2 | AA769075 |
| 99 | 203853_s_at | GRB2-associated binding protein 2 | GAB2 | NM_012296 |
| 100 | 203885_at | RAB21, member RAS oncogene family | RAB21 | NM_014999 |
| 101 | 203924_at | glutathione S-transferase A2 | GSTA1 | NM_000846 |
| 102 | 203953_s_at | Claudin 3 | CLDN3 | BE791251 |
| 103 | 203964_at | N-myc (and STAT) interactor | NMI | NM_004688 |
| 104 | 203974_at | haloacid dehalogenase-like hydrolase domain | HDHD1A | NM_012080 |
| | | containing 1A | | |
| 105 | 204014_at | dual specificity phosphatase 4 | DUSP4 | NM_001394 |
| 106 | 204036_at | endothelial differentiation, lysophosphatidic acid | EDG2 | AW269335 |
| | | G-protein-coupled receptor, 2 | | |
| 107 | 204037_at | | EDG2 | BF055366 |
| 108 | 204038_s_at | | EDG2 | NM_001401 |
| 109 | 204047_s_at | phosphatase and actin regulator 2 | PHACTR2 | AW295193 |
| 110 | 204049_s_at | | PHACTR2 | NM_014721 |
| 111 | 204136_at | collagen, type VII, alpha 1 | COL7A1 | NM_000094 |
| 112 | 204151_x_at | aldo-keto reductase family 1, member C1 | AKR1C1 | NM_001353 |
| 113 | 204154_at | cysteine dioxygenase, type I | CDO1 | NM_001801 |
| 114 | 204206_at | MAX binding protein | MNT | NM_020310 |
| 115 | 204268_at | S100 calcium-binding protein A2 | S100A2 | NM_005978 |
| 116 | 204326_x_at | metallothionein 1X | MT1X | NM_002450 |
| 117 | 204367_at | Sp2 transcription factor | SP2 | D28588 |
| 118 | 204379_s_at | fibroblast growth factor receptor 3 | FGFR3 | NM_000142 |
| 119 | 204385_at | kynureninase (L-kynurenine hydrolase) | KYNU | NM_003937 |
| 120 | 204388_s_at | monoamine oxidase A | MAOA | NM_000240 |
| 121 | 204455_at | bullous pemphigoid antigen 1 | BPAG1 | NM_001723 |
| 122 | 204460_s_at | RAD1 homolog | RAD1 | AF074717 |
| 123 | 204469_at | protein tyrosine phosphatase, receptor-type, Z | PTPRZ1 | NM_002851 |
| | | polypep 1 | | |
| 124 | 204493_at | BH3 interacting domain death agonist | BID | NM_001196 |
| 125 | 204532_x_at | UDP glycosyltransferase 1 family, polypep A9 | UGT1A9 | NM_021027 |
| 126 | 204542_at | sialyltransferase | SIAT7B | NM_006456 |
| 127 | 204547_at | RAB40B, member RAS oncogene family | RAB40B | NM_006822 |
| 128 | 204614_at | serine (or cysteine) proteinase inhibitor, clade B, | SERPINB2 | NM_002575 |
| | | mem 2 | | |
| 129 | 204621_s_at | nuclear receptor subfamily 4, group A, member 2 | NR4A2 | AI935096 |
| 130 | 204622_x_at | | NR4A2 | NM_006186 |
| 131 | 204633_s_at | nuclear mitogen- and stress-activated protein | RPS6KA5 | AF074393 |
| | | kinase-1 | | |
| 132 | 204636_at | collagen, type XVII, alpha 1 | COL17A1 | NM_000494 |
| 133 | 204672_s_at | ankyrin repeat domain 6 | ANKRD6 | NM_014942 |
| 134 | 204734_at | keratin 15 | KRT15 | NM_002275 |
| 135 | 204753_s_at | hepatic leukemia factor | HLF | AI810712 |
| 136 | 204754_at | hepatic leukemia factor | HLF | W60800 |
| 137 | 204755_x_at | hepatic leukemia factor | HLF | M95585 |
| 138 | 204855_at | serine (or cysteine) proteinase inhibitor, clade B, | SERPINB5 | NM_002639 |
| | | mem 5 | | |
| 139 | 204887_s_at | polo-like kinase 4 | PLK4 | NM_014264 |
| 140 | 204952_at | GPI-anchored metastasis-associated protein | C4.4A | NM_014400 |
| | | homolog | | |
| 141 | 204971_at | cystatin A (stefin A) | CSTA | NM_005213 |
| 142 | 205014_at | heparin-binding growth factor binding protein | FGFBP1 | NM_005130 |
| 143 | 205022_s_at | checkpoint suppressor 1 | CHES1 | NM_005197 |
| 144 | 205054_at | nebulin | NEB | NM_004543 |
| 145 | 205064_at | small proline-rich protein 1B | SPRR1B | NM_003125 |
| 146 | 205081_at | cysteine-rich protein 1 | CRIP1 | NM_001311 |
| 147 | 205141_at | angiogenin, ribonuclease, RNase A family, 5 | ANG | NM_001145 |
| 148 | 205157_s_at | keratin 17 | KRT17 | NM_000422 |
| 149 | 205176_s_at | integrin beta 3 binding protein (beta3-endonexin) | ITGB3BP | NM_014288 |
| 150 | 205206_at | Kallmann syndrome 1 sequence | KAL1 | NM_000216 |
| 151 | 205219_s_at | galactokinase 2 | GALK2 | NM_002044 |
| 152 | 205267_at | POU domain, class 2, associating factor 1 | POU2AF1 | NM_006235 |
| 153 | 205367_at | adaptor protein with pleckstrin homology and src | APS | NM_020979 |
| | | homology 2 domains | | |
| 154 | 205372_at | pleiomorphic adenoma gene 1 | PLAG1 | NM_002655 |
| 155 | 205450_at | phosphorylase kinase, alpha 1 (muscle) | PHKA1 | NM_002637 |
| 156 | 205490_x_at | gap junction protein, beta 3 | GJB3 | BF060667 |
| 157 | 205569_at | lysosomal-associated membrane protein 3 | LAMP3 | NM_014398 |
| 158 | 205595_at | desmoglein 3 | DSG3 | NM_001944 |
| 159 | 205618_at | proline rich Gla (G-carboxyglutamic acid) 1 | PRRG1 | NM_000950 |
| 160 | 205623_at | aldehyde dehydrogenase 3 | ALDH3A1 | NM_000691 |
| 161 | 205624_at | carboxypeptidase A3 (mast cell) | CPA3 | NM_001870 |
| 162 | 205789_at | CD1D antigen, d polypeptide | CD1D | NM_001766 |
| 163 | 205839_s_at | benzodiazapine receptor (peripheral) assoc pro 1 | BZRAP1 | NM_004758 |
| 164 | 205961_s_at | PC4 and SFRS1 interacting protein 1 | PSIP1 | NM_004682 |
| 165 | 205968_at | K+ voltage-gated channel, delayed-rectifier, | KCNS3 | NM_002252 |
| | | subfamily S, member 3 | | |
| 166 | 205969_at | arylacetamide deacetylase (esterase) | AADAC | NM_001086 |
| 167 | 206032_at | desmocollin 3, transcript variant Dsc3a | DSC3 | AI797281 |
| 168 | 206033_s_at | desmocollin 3, transcript variant Dsc3a | DSC3 | AI797281 |
| 169 | 206068_s_at | acyl-Coenzyme A dehydrogenase, long chain | ACADL | AI367275 |
| 170 | 206094_x_at | UDP glycosyltransferase 1 family, polypeptide A6 | UGT1A6 | NM_001072 |
| 171 | 206122_at | SRY-box 20 | SOX15 | NM_006942 |
| 172 | 206164_at | chloride channel, calcium activated, family mem 2 | CLCA2 | NM_006536 |
| 173 | 206165_s_at | chloride channel, calcium activated, family mem 2 | CLCA2 | NM_006536 |
| 174 | 206166_s_at | calcium-activated chloride channel-2 | CLCA2 | NM_006536 |
| 175 | 206300_s_at | parathyroid hormone-like hormone | PTHLH | NM_002820 |
| 176 | 206331_at | calcitonin receptor-like | CALCRL | NM_005795 |
| 177 | 206400_at | lectin, galactoside-binding, soluble, 7 | LGALS7 | NM_002307 |
| 178 | 206461_x_at | metallothionein 1H | MT1H | NM_005951 |
| 179 | 206561_s_at | aldo-keto reductase family 1, member B10 | AKR1B10 | NM_020299 |
| 180 | 206566_at | solute carrier family 7 (cationic amino acid | SLC7A1 | NM_003045 |
| | | transporter, y+ system), member 1 | | |
| 181 | 206581_at | basonuclin | BNC1 | NM_001717 |
| 182 | 206641_at | tumor necrosis factor receptor superfamily, mem 17 | TNFRSF17 | NM_001192 |
| 183 | 206653_at | Polymerase (RNA) III (DNA directed) polypep G | POLR3G | BF062139 |
| 184 | 206658_at | hypothetical protein MGC10902 | UPK3B | NM_030570 |
| 185 | 206756_at | carbohydrate (N-acetylglucosamine 6-O) | CHST7 | NM_019886 |
| | | sulfotransferase 7 | | |
| 186 | 206912_at | forkhead box E1 | FOXE1 | NM_004473 |
| 187 | 207029_at | KIT ligand | KITLG | NM_000899 |
| 188 | 207126_x_at | UDP glycosyltransferase 1 family, polypep A1 | UGT1A1 /// | NM_000463 |
| 189 | 207499_x_at | hypothetical protein FLJ10043 | SMAP-1 | NM_017979 |
| 190 | 207513_s_at | zinc finger protein 189 | ZNF189 | NM_003452 |
| 191 | 207620_s_at | calcium/calmodulin-dependent serine protein | CASK | NM_003688 |
| | | kinase | | |
| 192 | 207935_s_at | keratin 13 | KRT13 | NM_002274 |
| 193 | 208153_s_at | FAT tumor suppressor homolog 2 | FAT2 | NM_001447 |
| 194 | 208228_s_at | fibroblast growth factor receptor 2 | FGFR2 | M87771 |
| 195 | 208502_s_at | paired-like homeodomain transcription factor 1 | PITX1 | NM_002653 |
| 196 | 208539_x_at | small proline-rich protein 2B | SPRR2A | NM_006945 |
| 197 | 208581_x_at | metallothionein 1X | MT1X | NM_005952 |
| 198 | 208596_s_at | UDP glycosyltransferase 1 family, polypep A3 | UGT1A3 | NM_019093 |
| 199 | 208657_s_at | septin 9 | 9-Sep | AF142408 |
| 200 | 208692_at | ribosomal protein S3 | RPS3 | U14990 |
| 201 | 208737_at | ATPase, H+ transporting, lysosomal 13 kDa, V1 | ATP6V1G1 | BC003564 |
| | | subunit G isoform 1 | | |
| 202 | 208758_at | 5-aminoimidazole-4-carboxamide ribonucleotide | ATIC | D89976 |
| | | formyltransferase/IMP cyclohydrolase | | |
| 203 | 208798_x_at | golgin-67 | GOLGIN- | AF204231 |
| | | | 67 | |
| 204 | 208856_x_at | ribosomal protein, large, P0 | RPLP0 | BC003655 |
| 205 | 208870_x_at | ATP synthase, H+ transporting, mitochondrial F1 | ATP5C1 | BC000931 |
| | | complex, gamma polypeptide 1 | | |
| 206 | 208933_s_at | lectin, galactoside-binding, soluble, 8 | LGALS8 | AI659005 |
| 207 | 208935_s_at | lectin, galactoside-binding, soluble, 8 | LGALS8 | L78132 |
| 208 | 208950_s_at | aldehyde dehydrogenase 7 family, mem A1 | ALDH7A1 | BC002515 |
| 209 | 209009_at | esterase D/formylglutathione hydrolase | ESD | BC001169 |
| 210 | 209041_s_at | ubiquitin-conjugating enzyme E2G 2 | UBE2G2 | BG395660 |
| 211 | 209117_at | WW domain binding protein 2 | WBP2 | U79458 |
| 212 | 209122_at | adipose differentiation-related protein | ADFP | BC005127 |
| 213 | 209125_at | keratin 6A | KRT6A | J00269 |
| 214 | 209126_x_at | keratin 6 isoform K6f | KRT6B | L42612 |
| 215 | 209204_at | LIM domain only 4 | LMO4 | AI824831 |
| 216 | 209212_s_at | transcription factor BTEB2 | KLF5 | AB030824 |
| 217 | 209215_at | tetracycline transporter-like protein | TETRAN | L11669 |
| 218 | 209220_at | glypican 3 | GPC3 | L47125 |
| 219 | 209260_at | stratifin | SFN | BC000329 |
| 220 | 209296_at | protein phosphatase 1B (formerly 2C), magnesium- | PPM1B | AF136972 |
| | | dependent, beta isoform | | |
| 221 | 209309_at | zinc-alpha2-glycoprotein | AZGP1 | D90427 |
| 222 | 209339_at | seven in absentia homolog 2 | SIAH2 | U76248 |
| 223 | 209351_at | keratin 14 | KRT14 | BC002690 |
| 224 | 209380_s_at | CFTR/MRP, member 5 | ABCC5 | AF146074 |
| 225 | 209411_s_at | Golgi associated, gamma adaptin ear containing, | GGA3 | AW008018 |
| | | ARF binding protein 3 | | |
| 226 | 209446_s_at | Similar to hypothetical protein FLJ10803 | — | BC001743 |
| 227 | 209457_at | dual specificity phosphatase 5 | DUSP5 | U16996 |
| 228 | 209509_s_at | dolichyl-phosphate | DPAGT1 | BC000325 |
| 229 | 209587_at | hindlimb expressed homeobox protein backfoot | Bft | U70370 |
| 230 | 209647_s_at | IMAGE: 2972022 | SOCS5 | AW664421 |
| 231 | 209699_x_at | dihydrodiol dehydrogenase | AKR1C2 | U05598 |
| 232 | 209719_x_at | squamous cell carcinoma antigen 1 | SCCA1 | U19556 |
| 233 | 209720_s_at | serine (or cysteine) proteinase inhibitor, clade B | SERPINB3 | U19556 |
| | | (ovalbumin), member 3 | | |
| 234 | 209727_at | GM2 ganglioside activator | GM2A | M76477 |
| 235 | 209748_at | spastic paraplegia 4 | SPG4 | AB029006 |
| 236 | 209792_s_at | kallikrein 10 | KLK10 | BC002710 |
| 237 | 209800_at | keratin 16 | KRT16 | AF061812 |
| 238 | 209863_s_at | CUSP | TP73L | AF091627 |
| 239 | 209878_s_at | v-rel reticuloendotheliosis viral oncogene hom A, | RELA | M62399 |
| 240 | 209897_s_at | slit homolog 2 (Drosophila) | SLIT2 | AF055585 |
| 241 | 209959_at | nuclear receptor subfamily 4, group A, member 3 | NR4A3 | U12767 |
| 242 | 209963_s_at | erythropoietin receptor | EPOR | M34986 |
| 243 | 210020_x_at | NB-1 | CALML3 | M58026 |
| 244 | 210052_s_at | TPX2, microtubule-associated protein homolog | TPX2 | AF098158 |
| 245 | 210064_s_at | uroplakin 1B | UPK1B | NM_006952 |
| 246 | 210065_s_at | uroplakin Ib | UPK1B | NM_006952 |
| 247 | 210084_x_at | mast cell alpha II tryptase | — | AF206665 |
| 248 | 210133_at | chemokine (C—C motif) ligand 11 | CCL11 | D49372 |
| 249 | 210135_s_at | short stature homeobox 2 | SHOX2 | AF022654 |
| 250 | 210264_at | G protein-coupled receptor 35 | GPR35 | AF089087 |
| 251 | 210355_at | parathyroid-like protein | PTHLH | J03580 |
| 252 | 210406_s_at | RAB6A, member RAS oncogene family | RAB6A | AL136727 |
| 253 | 210505_at | alcohol dehydrogenase | ADH7 | U07821 |
| 254 | 210512_s_at | vascular endothelial growth factor | VEGF | AF022375 |
| 255 | 210829_s_at | single-stranded DNA binding protein 2 | SSBP2 | AF077048 |
| 256 | 210876_at | annexin A2 | ANXA2 | M62896 |
| 257 | 211002_s_at | tripartite motif protein TRIM29 beta | TRIM29 | AF230389 |
| 258 | 211105_s_at | nuclear factor of activated T-cells, cytoplasmic, | NFATC1 | U80918 |
| | | calcineurin-dependent 1 | | |
| 259 | 211194_s_at | p73H | TP73L | AB010153 |
| 260 | 211195_s_at | p51 delta | TP73L | AB010153 |
| 261 | 211272_s_at | diacylglycerol kinase, alpha 80 kDa | DGKA | AF064771 |
| 262 | 211361_s_at | hurpin | hurpin | AJ001696 |
| 263 | 211401_s_at | fibroblast growth factor receptor 2 | FGFR2 | AB030078 |
| 264 | 211452_x_at | clone FLB4816 PRO1252 | — | AF130054 |
| 265 | 211456_x_at | metallothionein 1H-like | — | AF333388 |
| 266 | 211474_s_at | serine (or cysteine) proteinase inhibitor, clade B | SERPINB6 | BC004948 |
| | | (ovalbumin), member 6 | | |
| 267 | 211527_x_at | vascular permeability factor | VEGF | M27281 |
| 268 | 211547_s_at | Miller-Dieker lissencephaly protein | LIS1 | L13387 |
| 269 | 211548_s_at | hydroxyprostaglandin dehydrogenase 15-(NAD) | HPGD | J05594 |
| 270 | 211596_s_at | leucine-rich repeats and immunoglobulin-like | LRIG1 | AB050468 |
| | | domains 1 | | |
| 271 | 211634_x_at | immunoglobulin heavy constant mu | IGHM | M24669 |
| 272 | 211635_x_at | IgM rheumatoid factor RF-TT1, VH chain | — | M24670 |
| 273 | 211653_x_at | pseudo-chlordecone | AKR1C2 | M33376 |
| 274 | 211689_s_at | transmembrane protease, serine 2 | TMPRSS2 | AF270487 |
| 275 | 211721_s_at | zinc finger proteins 551 | ZNF551 | BC005868 |
| 276 | 211734_s_at | IgE Fc, high affinity I, receptor for α polypep | FCER1A | BC005912 |
| 277 | 211756_at | parathyroid hormone-like hormone | PTHLH | BC005961 |
| 278 | 211834_s_at | p73Lp63p51p40KET | TP73L | AB042841 |
| 279 | 212061_at | KIAA0332 | SR140 | AB002330 |
| 280 | 212092_at | KIAA1051 | PEG10 | BE858180 |
| 281 | 212094_at | KIAA1051 | PEG10 | BE858180 |
| 282 | 212162_at | FLJ12811 | — | AK022873 |
| 283 | 212189_s_at | component of oligomeric Golgi complex 4 | COG4 | AK022874 |
| 284 | 212228_s_at | hypothetical protein DKFZp434K046 | DKFZP434K046 | AC004382 |
| 285 | 212236_x_at | cytokeratin 17 | KRT17 | Z19574 |
| 286 | 212252_at | Ca²⁺ calmodulin-dependent protein kinase kinase 2β | CAMKK2 | AA181179 |
| 287 | 212255_s_at | FLJ10822 fis | FLJ10822 | AK001684 |
| 288 | 212286_at | ankyrin repeat domain 12 | ANKRD12 | AW572909 |
| 289 | 212311_at | KIAA0746 protein | KIAA0746 | AA522514 |
| 290 | 212314_at | KIAA0746 protein | KIAA0746 | AB018289 |
| 291 | 212424_at | programmed cell death 11 | PDCD11 | AW026194 |
| 292 | 212441_at | KIAA0232 | KIAA0232 | D86985 |
| 293 | 212458_at | sprouty-related, EVH1 domain containing 2 | SPRED2 | H97931 |
| 294 | 212466_at | sprouty-related, EVH1 domain containing 2 | SPRED2 | AW138902 |
| 295 | 212570_at | KIAA0830 protein | KIAA0830 | AL573201 |
| 296 | 212573_at | KIAA0830 protein | KIAA0830 | AF131747 |
| 297 | 212595_s_at | DAZ associated protein 2 | DAZAP2 | AL534321 |
| 298 | 212599_at | autism susceptibility candidate 2 | AUTS2 | AK025298 |
| 299 | 212600_s_at | ubiquinol-cytochrome c reductase core protein II | UQCRC2 | AV727381 |
| 300 | 212662_at | poliovirus receptor | PVR | BE615277 |
| 301 | 212680_x_at | protein phosphatase 1, regulatory (inhibitor) | PPP1R14B | BE305165 |
| | | subunit 14B | | |
| 302 | 212836_at | polymerase (DNA-directed), delta 3, accessory | POLD3 | D26018 |
| | | subunit | | |
| 303 | 212841_s_at | PTPRF interacting protein, binding protein 2 | PPFIBP2 | AI692180 |
| 304 | 212864_at | CDP-diacylglycerol synthase (phosphatidate | CDS2 | Y16521 |
| | | cytidylyltransferase) 2 | | |
| 305 | 212914_at | chromobox homolog 7 | CBX7 | AV648364 |
| 306 | 212980_at | AHA1, activator of heat shock 90 kDa protein | AHSA2 | AL050376 |
| | | ATPase homolog 2 | | |
| 307 | 213023_at | utrophin | UTRN | NM_007124 |
| 308 | 213034_at | KIAA0999 protein | KIAA0999 | AB023216 |
| 309 | 213093_at | protein kinase C, alpha | PRKCA | AI471375 |
| 310 | 213199_at | DKFZP586P0123 protein | DKFZP586P0123 | AL080220 |
| 311 | 213325_at | poliovirus receptor-related 3 | PVRL3 | AA129716 |
| 312 | 213366_x_at | ATP synthase, H+ transporting, mitochondrial F1 | ATP5C1 | AV711183 |
| | | complex, gamma polypeptide 1 | | |
| 313 | 213425_at | wingless-type MMTV integration site family, | WNT5A | AI968085 |
| | | member 5A | | |
| 314 | 213440_at | RAB1A, member RAS oncogene family | RAB1A | AL530264 |
| 315 | 213471_at | nephronophthisis 4 | NPHP4 | AB014573 |
| 316 | 213490_s_at | mitogen-activated protein kinase kinase 2 | MAP2K2 | AI762811 |
| 317 | 213518_at | protein kinase C, iota | PRKCI | AI689429 |
| 318 | 213680_at | keratin 6A | KRT6B | AI831452 |
| 319 | 213700_s_at | Pyruvate kinase, muscle | PKM2 | AA554945 |
| 320 | 213721_at | SRY-box 2 | SOX2 | L07335 |
| 321 | 213722_at | SRY-box 2 | SOX2 | AW007161 |
| 322 | 213796_at | Small proline-rich protein SPRK | SPRR1A | AI923984 |
| 323 | 213808_at | 23688 clone | ADAM23 | BE674466 |
| 324 | 213843_x_at | accessory proteins BAP31BAP29 | SLC6A8 | AW276522 |
| 325 | 213880_at | leucine-rich repeat-containing G protein-coupled | LGR5 | AL524520 |
| | | receptor 5 | | |
| 326 | 213913_s_at | KIAA0984 protein | KIAA0984 | AW134976 |
| 327 | 214073_at | cortactin | CTTN | BG475299 |
| 328 | 214100_x_at | IMAGE: 1964520 | | AI284845 |
| 329 | 214260_at | COP9 constitutive photomorphogenic homolog | COPS8 | AI079287 |
| | | subunit 8 | | |
| 330 | 214441_at | syntaxin 6 | STX6 | NM_005819 |
| 331 | 214549_x_at | small proline-rich protein 1A | SPRR1A | NM_005987 |
| 332 | 214580_x_at | keratin 6B | KRT6B | AL569511 |
| 333 | 214680_at | neurotrophic tyrosine kinase, receptor, type 2 | NTRK2 | BF674712 |
| 334 | 214688_at | transducin-like enhancer of split 4 | TLE4 | BF217301 |
| 335 | 214735_at | phosphoinositide-binding protein PIP3-E | PIP3-E | AW166711 |
| 336 | 214812_s_at | KIAA0184 | KIAA0184 | D80006 |
| 337 | 214829_at | aminoadipate-semialdehyde synthase | AASS | AK023446 |
| 338 | 214965_at | hypothetical protein MGC26885 | MGC26885 | AF070574 |
| 339 | 215011_at | RNA, U17D small nucleolar | RNU17D | AJ006835 |
| 340 | 215030_at | G-rich RNA sequence binding factor 1 | GRSF1 | AK023187 |
| 341 | 215125_s_at | UDP glycosyltransferase 1 family, polypep A9 | UGT1A9 | AV691323 |
| 342 | 215189_at | keratin, hair, basic, 6 (monilethrix) | KRTHB6 | X99142 |
| 343 | 215354_s_at | proline-, glutamic acid-, leucine-rich protein 1 | PELP1 | BC002875 |
| 344 | 215372_x_at | Hypothetical protein LOC151878 | LOC151878 | AU146794 |
| 345 | 215382_x_at | mast cell alpha II tryptase | — | AF206666 |
| 346 | 215561_s_at | interleukin 1 receptor, type I | IL1R1 | AK026803 |
| 347 | 215786_at | Hepatitis B virus x associated protein | HBXAP | AK022170 |
| 348 | 215812_s_at | creatine transporter | SLC6A10 | U41163 |
| 349 | 216052_x_at | Artemin | ARTN | AF115765 |
| 350 | 216147_at | Septin 11 | 11-Sep | AL353942 |
| 351 | 216221_s_at | pumilio homolog 2 | PUM2 | D87078 |
| 352 | 216248_s_at | nuclear receptor subfamily 4, group A, member 2 | NR4A2 | S77154 |
| 353 | 216258_s_at | UV-B repressed sequence, HUR 7 | | BE148534 |
| 354 | 216263_s_at | chromosome 14 open reading frame 120 | C14orf120 | AK022215 |
| 355 | 216288_at | cysteinyl leukotriene receptor 1 | CYSLTR1 | AU159276 |
| 356 | 216412_x_at | IgG to Puumala virus G2, light chain V region | — | AF043584 |
| 357 | 216594_x_at | aldo-keto reductase family 1, member C1 | AKR1C1 | S68290 |
| 358 | 216603_at | solute carrier family 7, member 8 | — | AL365343 |
| 359 | 216722_at | VENT-like homeobox 2 pseudogene 1 | VENTX2P1 | AF164963 |
| 360 | 216918_s_at | bullous pemphigoid antigen 1 isoforms 1 and 3 | DST | AL096710 |
| 361 | 217003_s_at | tMDC II, isoform [d] | — | AJ132823 |
| 362 | 217097_s_at | hypothetical protein DKFZp564F013 | PHTF2 | AC004990 |
| 363 | 217165_x_at | metallothionein 1F (functional) | MT1F | M10943 |
| 364 | 217198_x_at | immunoglobulin heavy constant gamma 1 | IGHG1 | U80164 |
| 365 | 217227_x_at | immunoglobulin lambda locus | IGLVJC | X93006 |
| 366 | 217272_s_at | serine (or cysteine) proteinase inhibitor, clade B, | hurpin | AJ001698 |
| | | member 13 | | |
| 367 | 217312_s_at | collagen type VII intergenic region | COL7A1 | L23982 |
| 368 | 217388_s_at | kynureninase (L-kynurenine hydrolase) | KYNU | D55639 |
| 369 | 217418_x_at | membrane-spanning 4-domains, subfam A, mem 1 | MS4A1 | X12530 |
| 370 | 217480_x_at | similar to Ig kappa chain | LOC339562 | M20812 |
| 371 | 217528_at | chloride channel, calcium activated, family mem 2 | CLCA2 | BF003134 |
| 372 | 217622_at | chromosome 22 open reading frame 3 | C22orf3 | AA018187 |
| 373 | 217626_at | IMAGE: 3089210 | AKR1C2 /// | BF508244 |
| | | | AKR1C1 | |
| 374 | 217746_s_at | programmed cell death 6 interacting protein | PDCD6IP | NM_013374 |
| 375 | 217783_s_at | yippee-like | YPEL5 | NM_016061 |
| 376 | 217786_at | SKB1 homolog | SKB1 | NM_006109 |
| 377 | 217811_at | selenoprotein T | SELT | NM_016275 |
| 378 | 217841_s_at | protein phosphatase methylesterase-1 | PME-1 | NM_016147 |
| 379 | 217860_at | NADH dehydrogenase (ubiquinone) 1 alpha | NDUFA10 | NM_004544 |
| | | subcomplex, 10, | | |
| 380 | 217922_at | Mannosidase, alpha, class 1A, member 2 | MAN1A2 | AL157902 |
| 381 | 217994_x_at | hypothetical protein FLJ20542 | FLJ20542 | NM_017871 |
| 382 | 218070_s_at | GDP-mannose pyrophosphorylase A | GMPPA | NM_013335 |
| 383 | 218092_s_at | HIV-1 Rev binding protein | HRB | NM_004504 |
| 384 | 218192_at | inositol hexaphosphate kinase 2 | IHPK2 | NM_016291 |
| 385 | 218236_s_at | protein kinase D3 | PRKD3 | NM_005813 |
| 386 | 218238_at | GTP binding protein 4 | GTPBP4 | NM_012341 |
| 387 | 218239_s_at | GTP binding protein 4 | GTPBP4 | NM_012341 |
| 388 | 218288_s_at | hypothetical protein MDS025 | MDS025 | NM_021825 |
| 389 | 218305_at | importin 4 | IPO4 | NM_024658 |
| 390 | 218331_s_at | chromosome 10 open reading frame 18 | C10orf18 | NM_017782 |
| 391 | 218355_at | kinesin family member 4A | KIF4A | NM_012310 |
| 392 | 218384_at | calcium regulated heat stable protein 1 | CARHSP1 | NM_014316 |
| 393 | 218460_at | hypothetical protein FLJ20397 | FLJ20397 | NM_017802 |
| 394 | 218483_s_at | hypothetical protein FLJ21827 | FLJ21827 | NM_020153 |
| 395 | 218507_at | hypoxia-inducible protein 2 | HIG2 | NM_013332 |
| 396 | 218546_at | hypothetical protein FLJ14146 | FLJ14146 | NM_024709 |
| 397 | 218657_at | Link guanine nucleotide exchange factor II | RAPGEFL1 | NM_016339 |
| 398 | 218696_at | eukaryotic translation initiation factor 2-α kinase 3 | EIF2AK3 | NM_004836 |
| 399 | 218699_at | RAB7, member RAS oncogene family-like 1 | RAB7L1 | BG338251 |
| 400 | 218750_at | hypothetical protein MGC5306 | MGC5306 | NM_024116 |
| 401 | 218769_s_at | ankyrin repeat, family A (RFXANK-like), 2 | ANKRA2 | NM_023039 |
| 402 | 218796_at | hypothetical protein FLJ20116 | C20orf42 | NM_017671 |
| 403 | 218834_s_at | heat shock 70 kDa protein 5 (glucose-regulated | HSPA5BP1 | NM_017870 |
| | | protein, 78 kDa) binding protein 1 | | |
| 404 | 218957_s_at | hypothetical protein FLJ11848 | FLJ11848 | NM_025155 |
| 405 | 218960_at | transmembrane protease, serine 4 | TMPRSS4 | NM_016425 |
| 406 | 218962_s_at | hypothetical protein FLJ13576 | FLJ13576 | NM_022484 |
| 407 | 218990_s_at | small proline-rich protein 3 | SPRR3 | NM_005416 |
| 408 | 219129_s_at | hypothetical protein FLJ11526 | SAP30L | NM_024632 |
| 409 | 219132_at | pellino homolog 2 | PELI2 | NM_021255 |
| 410 | 219154_at | Ras homolog gene family, member F | RHOF | NM_024714 |
| 411 | 219155_at | phosphatidylinositol transfer protein, cytoplasmic 1 | PITPNC1 | NM_012417 |
| 412 | 219201_s_at | twisted gastrulation homolog 1 | TWSG1 | NM_020648 |
| 413 | 219217_at | hypothetical protein FLJ23441 | FLJ23441 | NM_024678 |
| 414 | 219241_x_at | hypothetical protein FLJ20515 | SSH3 | NM_017857 |
| 415 | 219245_s_at | hypothetical protein FLJ13491 | FLJ13491 | AI309636 |
| 416 | 219250_s_at | fibronectin leucine rich transmem protein 3 | FLRT3 | NM_013281 |
| 417 | 219347_at | nudix (nucleoside diphosphate linked moiety X)- | NUDT15 | NM_018283 |
| | | type motif 15 | | |
| 418 | 219389_at | hypothetical protein FLJ10052 | FLJ10052 | NM_017982 |
| 419 | 219554_at | Rh type C glycoprotein | RHCG | NM_016321 |
| 420 | 219582_at | opioid growth factor receptor-like 1 | OGFRL1 | NM_024576 |
| 421 | 219704_at | germ cell specific Y-box binding protein | YBX2 | NM_015982 |
| 422 | 219732_at | plasticity related gene 3 | PRG-3 | NM_017753 |
| 423 | 219741_x_at | zinc finger protein 552 | ZNF552 | NM_024762 |
| 424 | 219756_s_at | hypothetical protein FLJ22792 | POF1B | NM_024921 |
| 425 | 219854_at | zinc finger protein 14 (KOX 6) | ZNF14 | NM_021030 |
| 426 | 219936_s_at | G protein-coupled receptor 87 | GPR87 | NM_023915 |
| 427 | 219959_at | molybdenum cofactor sulfurase | MOCOS | NM_017947 |
| 428 | 219962_at | angiotensin I converting enzyme (peptidyl- | ACE2 | NM_021804 |
| | | dipeptidase A) 2 | | |
| 429 | 219995_s_at | hypothetical protein FLJ13841 | FLJ13841 | NM_024702 |
| 430 | 219997_s_at | COP9 constitutive photomorphogenic hom sub 7B | COPS7B | NM_022730 |
| 431 | 220046_s_at | cyclin L1 | CCNL1 | NM_020307 |
| 432 | 220177_s_at | transmembrane protease, serine 3 | TMPRSS3 | NM_024022 |
| 433 | 220285_at | chromosome 9 open reading frame 77 | C9orf77 | NM_016014 |
| 434 | 220466_at | hypothetical protein FLJ13215 | FLJ13215 | NM_025004 |
| 435 | 220664_at | small proline-rich protein 2C | SPRR2C | NM_006518 |
| 436 | 220668_s_at | DNA (cytosine-5-)-methyltransferase 3 beta | DNMT3B | NM_006892 |
| 437 | 221004_s_at | integral membrane protein 2C | ITM2C | NM_030926 |
| 438 | 221045_s_at | period homolog 3 | PER3 | NM_016831 |
| 439 | 221047_s_at | MAP/microtubule affinity-regulating kinase 1 | MARK1 | NM_018650 |
| 440 | 221050_s_at | GTP binding protein 2 | GTPBP2 | NM_019096 |
| 441 | 221064_s_at | chromosome 16 open reading frame 28 | C16orf28 | NM_023076 |
| 442 | 221096_s_at | hypothetical protein PRO1580 | PRO1580 | NM_018502 |
| 443 | 221234_s_at | BTB and CNC homology 1, basic leucine zipper | BACH2 | NM_021813 |
| | | transcription factor 2 | | |
| 444 | 221286_s_at | proapoptotic caspase adaptor protein | PACAP | NM_016459 |
| 445 | 221305_s_at | UDP glycosyltransferase 1 family, polypep A8 | UGT1A8 | NM_019076 |
| 446 | 221326_s_at | delta-tubulin | TUBD1 | NM_016261 |
| 447 | 221480_at | heterogeneous nuclear ribonucleoprotein D | HNRPD | BG180941 |
| 448 | 221513_s_at | UTP14, U3 small nucleolar ribonucleoprotein, | UTP14C/ | BC001149 |
| | | homolog C/homolog A | UTP14A | |
| 449 | 221514_at | U3 small nucleolar ribonucleoprotein, hom A | UTP14A | BC001149 |
| 450 | 221580_s_at | hypothetical protein MGC5306 | MGC5306 | BC001972 |
| 451 | 221597_s_at | HSPC171 protein | HSPC171 | BC003080 |
| 452 | 221622_s_at | uncharacterized hypothalamus protein HT007 | HT007 | AF246240 |
| 453 | 221649_s_at | peter pan homolog | PPAN | BC000535 |
| 454 | 221679_s_at | abhydrolase domain containing 6 | ABHD6 | AF225418 |
| 455 | 221770_at | ribulose-5-phosphate-3-epimerase | RPE | BE964473 |
| 456 | 221790_s_at | LDL receptor adaptor protein | ARH | AL545035 |
| 457 | 221795_at | Similar to hypothetical protein FLJ20093 | | AI346341 |
| 458 | 221796_at | Similar to hypothetical protein FLJ20093 | | AA707199 |
| 459 | 221854_at | ESTs | PKP1 | AI378979 |
| 460 | 221884_at | ecotropic viral integration site 1 | EVI1 | BE466525 |
| 461 | 243_g_at | microtubule-associated protein 4 | MAP4 | M64571 |
| 462 | 31846_at | ras homolog gene family, member D | RHOD | AW003733 |
| 463 | 33323_r_at | stratifin | SFN | X57348 |
| 464 | 33850_at | microtubule-associated protein 4 | MAP4 | W28892 |
| 465 | 34858_at | potassium channel tetramerisation domain | KCTD2 | D79998 |
| | | containing 2 | | |
| 466 | 37512_at | 3-hydroxysteroid epimerase | RODH | U89281 |
| 467 | 41037_at | TEA domain family member 4 | TEAD4 | U63824 |
| 468 | 41469_at | elafin | PI3 | L10343 |
| 469 | 44111_at | vacuolar protein sorting 33B | VPS33B | AI672363 |
| 470 | 49049_at | deltex 3 homolog | DTX3 | N92708 |
| 471 | 49077_at | protein phosphatase methylesterase-1 | PME-1 | AL040538 |
| 472 | 59625_at | nucleolar protein 3 | NOL3 | AI912351 |
| 473 | 65438_at | KIAA1609 protein | KIAA1609 | AA195124 |

### REFERENCES

Beer et al. (2002) “Gene-expression profiles predict survival of patients with lung adenocarcinoma” Nat Med 8:816-824
Brookes (1999) “The essence of SNPs” Gene 23:177-186
Kato et al. (2004) “A Randomized Trial of Adjuvant Chemotherapy with Uracil-Tegafur for Adenocarcinoma of the Lung” N Engl J Med 350:1713-1721
Kiernan et al. (1993) “Stage I non-small cell cancer of the lung results of surgical resection at Fairfax Hospital” Va Med Q 120:146-149
Kononen et al. (1998) “Tissue microarrays for high-throughput molecular profiling of tumor specimens” Nat Med 4:844-847
Mountain et al. (1987) “Lung cancer classification: the relationship of disease extent and cell type to survival in a clinical trials population” J Surg Oncol 35:147-156
Wingo et al. (1999) “Annual Report to the Nation on the Status of Cancer, 1973-1996, With a Special Section on Lung Cancer and Tobacco Smoking “J Natl Cancer Inst 91:675-690

1. A method of assessing lung cancer status comprising the steps of
a. obtaining a biological sample from a lung cancer patient; and
b. measuring Biomarkers associated with Marker genes corresponding to those selected from Table 1, Table 4, Table 5 or Table 7
wherein the expression levels of the Marker genes above or below pre-determined cut-off levels are indicative of lung cancer status.

2. A method of staging lung cancer patients comprising the steps of
a. obtaining a biological sample from a lung cancer patient; and
b. measuring Biomarkers associated with Marker genes corresponding to those selected from Table 1, Table 4, Table 5 or Table 7
wherein the expression levels of the Marker genes above or below pre-determined cut-off levels are indicative of the lung cancer stage.

3. The method of claim 2 wherein the stage corresponds to classification by the TNM system.

4. The method of claim 2 wherein the stage corresponds to patients with similar gene expression profiles.

5. A method of determining lung cancer patient treatment protocol comprising the steps of
a. obtaining a biological sample from a lung cancer patient; and
b. measuring Biomarkers associated with Marker genes corresponding to those selected from Table 1, Table 4, Table 5 or Table 7
wherein the expression levels of the Marker genes above or below pre-determined cut-off levels are sufficiently indicative of risk of recurrence to enable a physician to determine the degree and type of therapy recommended to prevent recurrence.

6. A method of treating a lung cancer patient comprising the steps of:
a. obtaining a biological sample from a lung cancer patient; and
b. measuring Biomarkers associated with Marker genes corresponding to those selected from Table 1, Table 4, Table 5 or Table 7
wherein the expression levels of the Marker genes above or below pre-determined cut-off levels are indicate a high risk of recurrence and;
c. treating the patient with adjuvant therapy if they are a high risk patient.

7. A method of determining whether a lung cancer patient is high or low risk of mortality comprising the steps of
a. obtaining a biological sample from a lung cancer patient; and
b. measuring Biomarkers associated with Marker genes corresponding to those selected from Table 4
wherein the expression levels of the Marker genes above or below pre-determined cut-off levels are sufficiently indicative of risk of mortality to enable a physician to determine the degree and type of therapy recommended.

8. The method of claim 1, 2, 5, 6 or 7 wherein the sample is prepared by a method are selected from the group consisting of bulk tissue preparation and laser capture microdissection.

9. The method of claim 8 wherein the bulk tissue preparation is obtained from a biopsy or a surgical specimen.

10. The method of claim 1, 2, 5, 6 or 7 further comprising measuring the expression level of at least one gene constitutively expressed in the sample.

11. The method of claim 1, 2, 5, 6 or 7 wherein the sample is obtained from a primary tumor.

12. The method of claim 1, 2, 5, 6 or 7 wherein the specificity is at least about 40%.

13. The method of claim 1, 2, 5, 6 or 7 wherein the sensitivity is at least at least about 80%.

14. The method of claim 1, 2, 5, 6 or 7 wherein the pre-determined cut-off levels are at least 1.5-fold over- or under-expression in the sample relative to benign cells or normal tissue.

15. The method of claim 1, 2, 5, 6 or 7 wherein the pre-determined cut-off levels have at least a statistically significant p-value over-expression in the sample having metastatic cells relative to benign cells or normal tissue.

16. The method of claim 28 wherein the p-value is less than 0.05.

17. The method of claim 1, 2, 5, 6 or 7 wherein gene expression is measured on a microarray or gene chip.

18. The method of claim 17 wherein the microarray is a cDNA array or an oligonucleotide array.

19. The method of claim 17 wherein the microarray or gene chip further comprises one or more internal control reagents.

18. The method of claim 1, 2, 5, 6 or 7 wherein gene expression is determined by nucleic acid amplification conducted by polymerase chain reaction (PCR) of RNA extracted from the sample.

20. The method of claim 18 wherein said PCR is reverse transcription polymerase chain reaction (RT-PCR).

21. The method of claim 20, wherein the RT-PCR further comprises one or more internal control reagents.

22. The method of claim 1, 2, 5, 6 or 7 wherein gene expression is detected by measuring or detecting a protein encoded by the gene.

23. The method of claim 22 wherein the protein is detected by an antibody specific to the protein.

24. The method of claim 1, 2, 5, 6 or 7 wherein gene expression is detected by measuring a characteristic of the gene.

25. The method of claim 24 wherein the characteristic measured is selected from the group consisting of DNA amplification, methylation, mutation and allelic variation.

26. A method of generating a lung cancer prognostic patient report comprising the steps of:
determining the results of any one of claims 1, 2, 5, 6 or 7; and
preparing a report displaying the results.

27. The method of claim 26 wherein the report contains an assessment of patient outcome and/or probability of risk relative to the patient population.

28. A patient report generated by the method according to claim 26.

29. A composition comprising at least one probe set selected from the group consisting of: Marker genes corresponding to those selected from Table 1, Table 4, Table 5 or Table 7.

30. A kit for conducting an assay to determine lung cancer prognosis in a biological sample comprising: materials for detecting isolated nucleic acid sequences, their complements, or portions thereof of a combination of genes selected from the group consisting of Marker genes corresponding to those selected from Table I, Table 4, Table 5 or Table 7.

31. The kit of claim 30 further comprising reagents for conducting a microarray analysis.

32. The kit of claim 30 further comprising a medium through which said nucleic acid sequences, their complements, or portions thereof are assayed.

33. Articles for assessing lung cancer status comprising: materials for detecting isolated nucleic acid sequences, their complements, or portions thereof of a combination of genes selected from the group consisting of Marker genes corresponding to those selected from Table 1, Table 4, Table 5 or Table 7.

34. The articles of claim 33 further comprising reagents for conducting a microarray analysis.

35. The articles of claim 34 further comprising a medium through which said nucleic acid sequences, their complements, or portions thereof are assayed.

36. A microarray or gene chip for performing the method of claim 1, 2, 5, 6 or 7.

37. The microarray of claim 36 comprising isolated nucleic acid sequences, their complements, or portions thereof of a combination of genes selected from the group consisting of Marker genes corresponding to those selected from Table 1, Table 4, Table 5 or Table 7.

38. The microarray of claim 37 wherein the measurement or characterization is at least 1.5-fold over- or under-expression.

39. The microarray of claim 37 wherein the measurement provides a statistically significant p-value over- or under-expression.

40. The microarray of claim 39 wherein the p-value is less than 0.05.

41. The microarray of claim 37 comprising a cDNA array or an oligonucleotide array.

42. The microarray of claim 37 further comprising or more internal control reagents.

43. A diagnostic/prognostic portfolio comprising isolated nucleic acid sequences, their complements, or portions thereof of a combination of genes selected from the group consisting of Marker genes corresponding to those selected from Table 1, Table 4, Table 5 or Table 7.

44. The portfolio of claim 43 wherein the measurement or characterization is at least 1.5-fold over- or under-expression.

45. The portfolio of claim 44 wherein the measurement provides a statistically significant p-value over- or under-expression.

46. The portfolio of claim 44 wherein the p-value is less than 0.05.
